(19) United States
(12) Patent Application Publication
(10) Pub. No.: US 2012/0108565 A1
(43) Pub. Date: May 3, 2012

(54) PYRROLE CARBOXYLIC ACID DERIVATIVES AS ANTIBACTERIAL AGENTS

(76) Inventors: Lalima Sharma, Chandigarh (IN); Jitendra A. Sattigeri, Gurgaon (IN); Narest Kumar, Gurgaon (IN); Ajay Yadav, Gurgaon (IN); Rijwan Momim, Ahmed Nagar (IN); Shahadat Ahmed, Karimganj (IN); Ian A. Cliffe, Gurgaon (IN); Pradip Kumar Bhatnagar, Exton, PA (US); Sanjay Ghosh, Guwahati (IN); V. Samuel Raj, Sivakasi (IN); Dilip J. Upadhyay, Thune (IN)

(21) Appl. No.: 13/056,876

(22) PCT Filed: Jul. 30, 2009
(86) PCT No.: PCT/IB2009/053331

§ 371 Date: Apr. 21, 2011

(30) Foreign Application Priority Data
Jul. 30, 2008 (IN) 1809/DEL/2008

(51) Int. Cl. A61K 31/403; C07D 401/14; C07D 403/12; C07D 413/14; A61K 31/427; A61K 31/4439; A61K 31/428; A61K 31/5377; A61K 31/506; A61P 31/04; A61P 11/00; A61P 17/00; A61P 13/00; A61P 19/08; C07D 405/14; C07D 407/14; C07D 403/14; A61K 31/422; A61K 31/454; A61K 31/496; C07D 417/14

(52) U.S. Cl. 514/210.18; 548/181; 546/276.7; 548/468; 548/159; 544/122; 544/324; 514/370; 514/339; 514/414; 514/367; 514/235.2; 514/275; 548/236; 546/270.4; 546/269.7; 546/200; 544/133; 544/369; 514/377; 514/323; 514/254.02

(57) ABSTRACT

The present invention provides DNA Gyrase and/or Topo IV inhibitors of Formula (I), which can be used as antibacterial agents. Compounds disclosed herein can be used for treating or preventing conditions caused by or contributed by gram positive, gram negative and anaerobic bacteria, more particularly against, for example, Staphylococci, Streptococci, Enterococci, Haemophilus, Pseudomonus spp., Acenetobacter spp., Mvraxalla spp., Chlamydia spp., Mycoplasma spp., Legionella spp., Mycobacterium spp., Helicobacter, Clostridium spp., Bacteroides spp., Cotynebacterium, Bacillus spp., Enterobactericeae (E. coli, Klebsiella spp., Proteus spp., etc.) or any combination thereof. Also provided, are processes for preparing compounds disclosed herein, pharmaceutical compositions containing compounds disclosed herein, and methods of treating bacterial infections.

### FIELD OF INVENTION

The present invention provides DNAGyrase and/or Topo IV inhibitors, which can be used as antibacterial agents. Compounds disclosed herein can be used for treating or preventing conditions caused by or contributed by gram positive, gram negative and anaerobic bacteria, more particularly against, for example, Staphylococci, Streptococci, Enterococci, Haemophilus, Pseudomonas spp., Acenetobacter spp., Moraxalla spp., Chlamydia spp., Mycoplasma spp., Legionella spp., Mycobacterium spp., Helicobacter, Clostridium spp., Bacteroides spp., Corynebacterium, Bacillus spp., Enterobactericeae (E. coli, Klebsiella spp or Proteus spp) or any combination thereof. Also provided, are processes for preparing compounds disclosed herein, pharmaceutical compositions containing compounds disclosed herein, and methods of treating bacterial infections.

### BACKGROUND OF THE INVENTION

Emergence of drug resistance to the existing drugs and increasing need of drugs to treat the endemic and epidemic diseases have driven the major pharmaceutical companies to discover novel antibacterial targets. The international microbiological community continues to express serious concern that the evolution of bacterial resistance could result in strains against which currently available antibacterial agents will be ineffective. In general, bacterial pathogens may be classified either gram-positive or gram-negative pathogens. The antibiotics, which are effective against both types of organisms, are called as broad-spectrum antibiotics. Gram-positive organisms are particularly important for example, Staphylococci, Enterococci, Streptococci and Mycobacterium because of the development of resistant strain that are both difficult to treat and difficult to eradicate from the hospital environment once established.

The fluoroquinolones have been used to treat a great variety of infection including respiratory tract infections (Smith H. J. et al., “J. Antimicrobial Chemother.” 2002, 49, 893-895). As a result of their wide spectrum of activity, quinolones have been extensively used. Because of this high level use and to some degree of misuse, it has caused rapid development of bacterial resistance to these agents. With the approval of the three most recent antibacterial agents, linezolid in 2000, daptomycin in 2004 and telithromycin in 2002-04, three new classes of agents have been introduced into the market. However, resistance has already been reported for all these three agents, thus providing an opportunity for additional agents in these classes to overcome the new resistance identified. In addition, new targets should be explored to avoid these resistance already reported in the existing classes of antibiotics.

Methicillin resistant Staphylococcus aureus (MRSA) infections constitute the single most important cause of health care-associated infections, increasing lengths of hospital stay, severity of illness, deaths and costs. Although these infections occurred primarily in hospitals, they are becoming increasingly common in communities nationwide, especially where groups of people are in close quarters, including military facilities, sports teams and prisons. MRSA infection is more difficult to treat because the bacteria are resistant to β-lactam antibiotics such as methicillin, oxacillin, penicillin and amoxicillin. They are also resistant to macrolides, fluoroquinolones, clindamycin and trimethoprim/sulfamethoxazole. These infections can progress to life-threatening blood or bone infections because there are fewer effective antibiotics available for treatment. The treatment for MRSA may be longer, more expensive and more complicated, and infections can reappear frequently.

The glycopeptide antibiotics, teicoplanin and vancomycin are currently the mainstay of treatment of infections with MRSA. However, strains of MRSA have emerged to show intermediate susceptibility to glycopeptide antibiotics (GISA), or vancomycin (VISA). Oxazolidinones are new class of molecules active against MRSA and linezolid is the only drug available in the market. However, the toxicity of linezolid is the major issue and linezolid resistance has started emerging.

As a result, the need to combat drug-resistant bacteria and the increasing failure of the available drugs, there has been resurgent interest in discovering new antibiotics particularly those with either a novel mechanism of action and/or containing new pharmacophoric groups. One attractive strategy for the development of new antibiotics is to inhibit DNA gyrase, a bacterial enzyme necessary for DNA replication and therefore, necessary for bacterial cell growth and division. Gyrase activity is also associated with events in DNA transcription, repair and recombination.

DNA topoisomerases are enzymes that control the topology of the DNA in cells. DNA gyrase and topoisomerase IV are essential enzymes and play important role in DNA replication and compaction (Drlica and Zhao, “Microbiol Mol Biol Rev.” 1997, 61, 377-92). DNA supercoiling activity is essential in all bacteria but not found in humans and it is an ideal target for antibacterials. Gyrase catalyzes the conversion of relaxed, closed circular duplex DNA to a negatively superhelical form, which is more favorable for recombination. The mechanism of supercoiling reaction involves the wrapping of gyrase around a region of the DNA, double strand breaking in that region, passing a second region of the DNA through the break and rejoining the broken strands (Maxwell, A. “Trends Microbiol” 1997, 5, 102-109; Drlica and Zhao, “Microbiol Mol Biol Rev.” 1997, 61, 377-92). The supercoiling reaction is driven by the binding of ATP to gyrase and the ATP is then hydrolyzed during the reaction (Levine C. et al., “Biochim Biophys Acta” 1998, 1400, 29-43). This ATP binding and subsequent hydrolysis cause conformational changes in the DNA-bound gyrase that are necessary for its activity.

Bacterial DNA gyrase is a 400 kilodalton protein consisting of A₂B₂ heterotetramer (Maxwell, A. “Trends Microbiol” 1997, 5, 102-109). The A subunit (gyrA) comprises an N-terminal domain involved in DNA cleavage and religation and a C-terminal DNA-wrapping domain. The B-subunit (gyrB) contains a ATP hydrolysis at N-terminal domain and C-terminal domain interacts with both Gyrase A and DNA. Another conserved and essential type-II topoisomerase in bacteria, called TopoIV, is primarily responsible for separating the linked closed circular bacterial chromosomes produced in replication. This enzyme relaxes the supercoiled DNA. Topoisomerase IV is a C₂E₂ enzyme, encoded by parC and parE. These subunits parC and parE are highly identical to GyrA and GyrB, respectively. In S. aureus, the identity between GyrB and parE is 52%, where as the identity between GyrA and B is only 5%. The overall sequence identity between gyrase and topoisomerase IV in different bacterial species is high. Therefore, the compounds that target bacterial type-II topoisomerases have the potential to inhibit two targets in cell i.e. DNA gyrase and Topo IV; as is the case in present invention.

The continuous emergence of antibiotic resistance demands that novel classes of antibiotics to be developed. In pursuit of that goal, the present invention discloses some substituted azabicyclo compounds useful for the treatment of bacterial infection. WO2005026149 discloses piperidine derivatives as Gyrase B inhibitors. WO2007007281 discloses azabicyclo derivatives that are muscarinic receptor antagonists. WO2005005420 discloses cyclopropyl group substituted oxazolidinone antibiotics that are effective against aerobic and anaerobic pathogens.

### SUMMARY OF THE INVENTION

The present invention provides azabicyclo compounds having DNA Gyrase and/or Topo IV inhibitory activity. The compounds can be used in the treatment or prevention of bacterial infection. Also, provided are processes for synthesizing such compounds.

The compounds of the said invention exhibit activity against strains of Gram-positive, Gram-negative and anaerobic bacteria. Therefore, the compounds of present invention are useful for the treatment of pathologic condition arisen from bacterial infection or contamination.

Pharmaceutical compositions containing such compounds are provided together with the pharmaceutically acceptable carriers or diluents, which can be used for the treatment or prevention of bacterial infections. These pharmaceutical compositions may be administered or coadministered by a wide variety of routes including, for example, oral, topical, rectal, intranasal or by parenteral route. The composition may also be administered or co-administered in slow release dosage forms.

Although, the specific enantiomers have been shown by way of examples, the racemates, diastereomers, N-oxides, polymorphs, pharmaceutically acceptable salts, pharmaceutically acceptable solvates, co-crystals, prodrugs and metabolites having the same type of activity, are also provided. The pharmaceutical compositions comprising the compounds, their metabolites, racemates, enantiomers, N-oxides, polymorphs, solvates, co-crystals, prodrugs or pharmaceutically acceptable salts thereof, in combination with a pharmaceutically acceptable carrier and optionally included excipients are also included.

The therapeutically effective amounts of one or more compounds of the present invention can be used in combination with one or more other therapeutic agents, for example, protein synthesis inhibitors, aminoglycosides, cell wall synthesis inhibitors (glycopeptides, beta-lactams, etc.), RNA and DNA synthesis inhibitors or fatty acid synthesis inhibitors.

Other objects will be set forth in accompanying description and in the part will be apparent from the description or may be learnt by the practice of the invention.

### DETAILED DESCRIPTION OF THE INVENTION

In accordance with one aspect of the invention, are provided compounds having the structure of Formula I

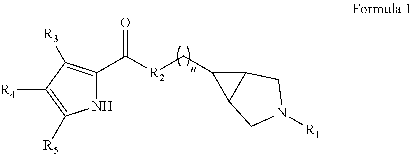

and its pharmaceutically acceptable salts, pharmaceutically acceptable solvates, co-crystals, enantiomers, diastereomers, polymorphs, prodrugs, metabolites or N-oxides
wherein,
n can be 0-2;
R₁ can be cycloalkyl, aryl, heteroaryl or heterocyclyl which may optionally be substituted with 1-3 substituent independently selected from alkyl, —(CH₂)ₓ—C(═O)OR_{d}, —(CH₂)ₓ—CONR_{f}R_{q}, cyano, halo, CH₂—OH, —CO-cycloalkyl, —CO-heteroaryl, —(CH₂)ₓ—CO-heterocyclyl, —CF₃, —OCF₃ or R₈—(R₉)ₘ;
wherein
R₈ can be heteroaryl, heterocyclyl, aryl, alkyl, cycloalkyl, (C₁₋₆)alkyl-cycloalkyl, (C₁₋₆)alkyl-heteroaryl, (C₁₋₆)alkyl-heterocyclyl, (C₁₋₆)alkyl-aryl;
R₉ can be hydrogen, alkyl, hydroxy, —C(═O)OR_{d}, halo, —NR_{f}R_{q}, —CO-heteroaryl, alkoxy, —NHCORₐ, aryl, heteroaryl, heterocyclyl, cycloalkyl, aryloxy, heteroaryloxy, heterocyclyloxy, aralkyloxy, heteroarylalkyloxy, heterocyclylalkyloxy, —CF₃, —OCF₃, alkylcarbonyl, —CONR_{f}R_{q}, —NHCONR_{f}R_{q}, —NH(CH₂)ₓC(═O)OR_{d}, —NH(CH₂)ₓcycloalkyl, —NH(CH₂)ₓheteroaryl, —NH(CH₂)ₓheterocyclyl or —NH(CH₂)ₓaryl;
   Rₐ can be alkyl, alkoxy or NR_{f}R_{q};
   R_{d} can be hydrogen or alkyl;
      R_{f} and R_{q} can independently be hydrogen, alkyl, alkoxy, cycloalkyl, aryl, heteroaryl and heterocyclyl, (C(C₁₋₆)alkyl-heteroaryl, (C₁₋₆)alkyl-heterocyclyl, (C₁₋₆)alkyl-aryl, (CH₂)ₓ—OH, (CH₂)ₓ—C(═O)OR_{d}, heteroarylalkyl, heteroaryl-heteroaryl, heterocyclylalkyl, arylalkyl, cycloalkylalkyl or R_{f} and R_{q} together with ‘N’ to which they are attached form a ring which optionally may contain heteroatom selected from N, O and S;
x can be 0-2;
m can be 1-4;
   R₂ can be O, NH or CHR″ (wherein R″ can be hydrogen, alkyl, cyano, nitro, amino, hydroxyl or alkoxy);
   R₃, R₄, R₅ can independently be hydrogen, alkyl, alkenyl, alkynyl, halogen, cyano, amino, hydroxy, alkoxy, carbonyl, thiocarbonyl, oxo, cycloalkyl, aryl, heteroaryl, heterocyclyl, arylalkyl, heteroarylalkyl, cycloalkylalkyl or heterocyclylalkyl or R₃ and R₄ or R₄ and R₅ taken together with the carbon atoms to which they are attached can form an aromatic or non-aromatic ring, which optionally may contain heteroatom selected from N, O and S.

In another embodiment, current invention provide a compound of Formula Ia

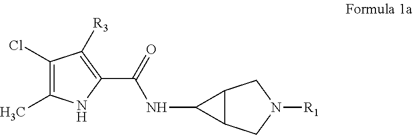

and its pharmaceutically acceptable salts, pharmaceutically acceptable solvates, co-crystals, enantiomers, diastereomers, polymorphs, prodrugs, metabolites or N-oxides
wherein,
R₃ is selected from hydrogen or chloro;
R₁ can be aryl, heteroaryl, which may optionally be substituted with 1-3 substituent independently selected from alkyl, —(CH₂)ₓ—C(═O)OR_{d}, —(CH₂)ₓ—CONR_{f}R_{q}, cyano, halo, CH₂—OH, —CO-cycloalkyl, —CO-heteroaryl, —(CH₂)ₓ—CO-heterocyclyl, —CF₃, —OCF₃ or R₈—(R₉)ₘ,
wherein
R₈ can be heteroaryl, heterocyclyl, aryl, alkyl, cycloalkyl, (C₁₋₆)alkyl-cycloalkyl, (C₁₋₆)alkyl-heteroaryl, (C₁₋₆)alkyl-heterocyclyl or (C₁₋₆)alkyl-aryl;
R₉ can be hydrogen, alkyl, hydroxy, —C(═O)OR_{d}, halo, —NR_{f}R_{q}, —CO-heteroaryl, alkoxy, —NHCORₐ, aryl, heteroaryl, heterocyclyl, cycloalkyl, aryloxy, heteroaryloxy, heterocyclyloxy, aralkyloxy, heteroarylalkyloxy, heterocyclylalkyloxy, —CF₃, —OCF₃, alkylcarbonyl, —CONR_{f}R_{q}, NHCONR_{f}R_{q}, —NH(CH₂)ₓC(═O)OR_{d}, —NH(CH₂)ₓcycloalkyl, —NH(CH₂)ₓheteroaryl, —NH(CH₂)ₓheterocyclyl or —NH(CH₂)ₓaryl;
   Rₐ can be alkyl, alkoxy or NR_{f}R_{q};
   R_{d} can be hydrogen or alkyl;
   R_{f} and R_{q} can independently be hydrogen, alkyl, alkoxy, cycloalkyl, aryl, heteroaryl, heterocyclyl, (C₁₋₆)alkyl-cycloalkyl, (C₁₋₆)alkyl-heteroaryl, (C₁₋₆)alkyl-heterocyclyl, (C₁₋₆)alkyl-aryl, (CH₂)ₓ—OH, (CH₂)ₓ—C(═O)OR_{d}, heteroarylalkyl, heteroaryl-heteroaryl, heterocyclylalkyl, arylalkyl, cycloalkylalkyl or R_{f} and R_{q} together with ‘N’ to which they are attached can form a ring which optionally may contain heteroatom selected from N, O and S;
x can be 0-2;
m can be 1-4.

In one embodiment, said alkyl is selected from a branched or unbranched saturated hydrocarbon chain having 1 to 20 carbon atoms, for example, methyl, ethyl, n-propyl, iso-propyl, n-butyl, iso-butyl, t-butyl, n-hexyl and the like.

In other embodiment, said aryl is selected from a single aromatic ring, or polycyclic (fused) ring containing 5 to 15 carbon atoms wherein at least one of the rings is aromatic, optionally substituted with 1 to 3 substituents. The said aryl group can be selected from phenyl, naphthyl, anthracenyl and the like.

In another embodiment, said heteroaryl is selected from a 5 to 6 membered monocyclic or a 8 to 16 membered polycylic aromatic group containing at least one heteroatom, independently selected from the group consisting of N, O and S. It may optionally be substituted with 1 to 8 substituents. Examples of heteroaryl groups are pyridinyl, quinolinyl, oxazolyl, imidazolyl, pyrrolyl, 1,2,3-triazolyl, 1,2,4-triazolyl, tetrazolyl, thiazolyl, oxadiazolyl, benzoimidazolyl, thiadiazolyl, pyrazolyl, furanyl, azetidinyl, pyridazinyl, pyrimidinyl, thienyl, isoxazolyl, triazinyl, furanyl, benzofuranyl, indolyl, benzothiazolyl, benzoxazolyl, thiophenyl, benzimidazolyl, quinolinyl, benzodioxolyl, indazolyl and the like.

In another embodiment, said heterocyclyl is a non-aromatic monocyclic or polycyclic (multiple condensed, Spiro or bridged) cycloalkyl group of 5 to 16 atoms in which 1 to 4 carbon atoms in the ring are replaced by a heteroatom selected from the group comprising of O, S and N, wherein the optionally-fused ring may, in turn, be saturated or unsaturated and may further contain 1-4 heteroatoms selected from the group comprising of N, O, and S. It may be optionally substituted with one or more of the substituents. Examples of heterocyclyl groups are thiazolidinyl, oxazolidinyl, tetrahydrofuranyl, tetrahydropyranyl, dihydrofuranyl, dihydroisoxazolyl, dihydrobenzofuryl, azabicyclohexyl, azabicyclooctanyl, dihydroindolyl, piperidinyl or piperazinyl, tetrahydroquinolinyl, tetrahydrothiopyranyl, pyrrolidinyl, morpholinyl, piperizinyl, azepinyl, azetidinyl, aziridinyl, tetrahydropyridinyl, benzthiazinyl, benzoxazinyl, isoindolinyl, phenoxazine and the like.

In another embodiment, cycloalkyl is a cyclic alkyl group of 3 to 20 carbon atoms having a monocyclic ring or polycyclic (fused, Spiro and bridged rings) ring, which may optionally contain one or more olefinic bonds. The cycloalkyl groups of the present invention can be selectred from single ring structures such as cyclopropyl, cyclobutyl, cyclopentyl, cyclohexyl, cycloheptyl, cyclooctyl, cyclobutenyl, cyclopentenyl, cyclohexenyl and the like, and multiple ring structures such as adamantyl, bicyclo[2.2.1]heptanyl and the like.

Said alkyl, aryl, heteroaryl, heterocyclyl and cycloalkyl of the present invention can further be substituted with alkyl, hydroxy, alkoxy, halo, azido, cyano, nitro, —C(═O)—R_{λ}, —SR_{λ}, —CF₃, —OCF₃, —C(═O)OR_{d}, —CONR_{f}R_{q}, —NHCORₐ, —O—C(═O)NR_{λ}R_{π}, C(═S)—R_{λ}, —NHSO₂R_{f}, —SO₂NHR_{f}, —NR_{f}R_{q}— wherein R_{λ} and R_{π} are independently selected from hydrogen, alkyl, cycloalkyl, aryl, heterocyclyl, heteroaryl and Rₐ, R_{d}, R_{f} and R_{q} are as defined earlier.

In one aspect, the invention encompasses compounds that include, for example,
2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxylic acid Lithium salt (Compound No. 1),
2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 2),
2-[(1R,5S,6s)-6-{[(4-chloro-5-methyl-1H-pyrrol-2-yl)carbonyl]amino}-3-azabicyclo[3.1.0]hex-3-yl]pyridine-3-carboxamide (Compound No. 3),
2-[(1R,5S,6s)-6-{[(3,4-dichloro-5-methyl-1H-pyrrol-2-yl)carbonyl]amino}-3-azabicyclo[3.1.0]hex-3-yl]pyridine-3-carboxamide (Compound No. 4),
3,4-Dichloro-N-[(1R,5S,6s)-3-(5-cyanopyridin-2-yl)-3-azabicyclo[3.1.0]hex-6-yl]-5-methyl-1H-pyrrole-2-carboxamide (Compound No. 5),
2-[(1R,5S,6s)-6-{[(4-chloro-5-methyl-1H-pyrrol-2-yl)carbonyl]amino}-3-azabicyclo [3.1.0]hex-3-yl]-4-methyl-1,3-thiazole-5-carboxylic acid Lithium salt (Compound No. 6),
2-Chloro-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylic acid Lithium salt (Compound No. 7),
2-Chloro-6-[(1R,5S,6s)-6-{[(3,4-dichloro-5-methyl-1H-pyrrol-2-yl)carbonyl]amino}-3-azabicyclo[3.1.0]hex-3-yl]pyridine-4-carboxylic acid Lithium salt (Compound No. 8),
6-[(1R,5S,6s)-6-{[(3,4-dichloro-5-methyl-1H-pyrrol-2-yl)carbonyl]amino}-3-azabicyclo[3.1.0]hex-3-yl]pyridine-2-carboxylic acid Lithium salt (Compound No. 9),
6-[(1R,5S,6s)-6-{[(3,4-dichloro-5-methyl-1H-pyrrol-2-yl)carbonyl]amino}-3-azabicyclo[3.1.0]hex-3-yl]pyridine-3-carboxylic acid Lithium salt (Compound No. 10),
6-[(1R,5S,6s)-6-{[(3,4-dichloro-5-methyl-1H-pyrrol-2-yl)carbonyl]amino}-3-azabicyclo[3.1.0]hex-3-yl]-2-morpholin-4-ylpyrimidine-4-carboxylic acid Lithium salt (Compound No. 11),
{2-[(1R,5S,6s)-6-{[(3,4-dichloro-5-methyl-1H-pyrrol-2-yl)carbonyl]amino}-3-azabicyclo[3.1.0]hex-3-yl]-1,3-thiazol-4-yl}acetic acid Lithium salt (Compound No. 12),
5-[(1R,5S,6s)-6-{[(3,4-dichloro-5-methyl-1H-pyrrol-2-yl)carbonyl]amino}-3-azabicyclo[3.1.0]hex-3-yl]furan-2-carboxylic acid Lithium salt (Compound No. 13),
5-[(1R,5S,6s)-6-{[(3,4-dichloro-5-methyl-1H-pyrwl-2-yl)carbonyl]amino}-3-azabicyclo[3.1.0]hex-3-yl]thiophene-2-carboxylic acid Lithium salt (Compound No. 14),
6-[(1R,5S,6s)-6-{[(3,4-dichloro-5-methyl-1H-pyrrol-2-yl)carbonyl]amino}-3-azabicyclo[3.1.0]hex-3-yl]naphthalene-2-carboxylic acid Lithium salt (Compound No. 15),
2-(4-Carboxypiperidin-1-yl)-6-[(1R,5S,6s)-6-{[(3,4-dichloro-5-methyl-1H-pyrrol-2-yl) carbonyl]amino}-3-azabicyclo[3.1.0]hex-3-yl]pyrimidine-4-carboxylic acid Lithium salt (Compound No. 16),
{4-[(1R,5S,6s)-6-{[(3,4-dichloro-5-methyl-1H-pyrrol-2-yl)carbonyl]amino}-3-azabicyclo[3.1.0]hex-3-yl]phenyl}acetic acid Lithium salt (Compound No. 17),
3-[(1R,5S,6s)-6-{[(3,4-dichloro-5-methyl-1H-pyrrol-2-yl)carbonyl]amino}-3-azabicyclo[3.1.0]hex-3-yl]benzoic acid Lithium salt (Compound No. 18),
2-(4-Aminopiperidin-1-yl)-6-[(1R,5S,6s)-6-{[(3,4-dichloro-5-methyl-1H-pyrrol-2-yl) carbonyl]amino}-3-azabicyclo[3.1.0]hex-3-yl]pyrimidine-4-carboxylic acid trifluoroacetate salt (Compound No. 19),
2-[(1R,5S,6s)-6-{[(3,4-dichloro-5-methyl-1H-pyrrol-2-yl)carbonyl]amino}-3-azabicyclo[3.1.0]hex-3-yl]-4-propyl-1,3-thiazole-5-carboxylic acid Lithium salt (Compound No. 20),
4-Benzyl-2-{[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 21),
2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxamide (Compound No. 22),
2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-4-carboxylic acid (Compound No. 23),
2-[(1R,5S,6s)-6-{[(3,4-dichloro-5-methyl-1H-pyrrol-2-yl)carbonyl]amino}-3-azabicyclo[3.1.0]hex-3-yl]-4-(methoxymethyl)-1,3-thiazole-5-carboxylic acid (Compound No. 24),
2-[(1R,5S,6s)-6-{[(3,4-dichloro-5-methyl-1H-pyrrol-2-yl)carbonyl]amino}-3-azabicyclo[3.1.0]hex-3-yl]-4-pentyl-1,3-thiazole-5-carboxylic acid (Compound No. 25),
2-[(1R,5S,6s)-6-{[(3,4-dichloro-5-methyl-1H-pyrrol-2-yl)carbonyl]amino}-3-azabicyclo[3.1.0]hex-3-yl]-4-phenyl-1,3-thiazole-5-carboxylic acid (Compound No. 26),
2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-4,5-dicarboxylic acid Sodium salt (Compound No. 27),
6-[(1R,5S,6s)-6-{[(3,4-dichloro-5-methyl-1H-pyrrol-2-yl)carbonyl]amino}-3-azabicyclo[3.1.0]hex-3-yl]-2-(piperidin-1-yl)pyrimidine-4-carboxylic acid Lithium salt (Compound No. 28),
2-{4-[(tert-butoxycarbonyl)amino]piperidin-1-yl}-6-{[(1R,5S,6s)-6-{[(3,4-dichloro-5-methyl-1H-pyrrol-2-yl)carbonyl]amino-3-azabicyclo[3.1.0]hex-3-yl]pyrimidine-4-carboxylic acid Lithium salt (Compound No. 29),
6-[(1R,5S,6s)-6-{[(3,4-dichloro-5-methyl-1H-pyrrol-2-yl)carbonyl]amino}-3-azabicyclo[3.1.0]hex-3-yl]-2-(pyrrolidin-1-yl)pyrimidine-4-carboxylic acid Lithium salt (Compound No. 30),
3,4-Dichloro-5-methyl-N-{(1R,5S,6s)-3-[2-(pyrrolidin-1-yl)-6-(pyrrolidin-1-ylcarbonyl)pyrimidin-4-yl]-3-azabicyclo[3.1.0]hex-6-yl}-1H-pyrrole-2-carboxamide (Compound No. 31),
N-[(1R,5S,6s)-3-(1,3-benzothiazol-2-yl)-3-azabicyclo[3.1.0]hex-6-yl]-3,4-dichloro-5-methyl-1H-pyrrole-2-carboxamide (Compound No. 32),
2-[(1R,5S,6s)-6-1 [(3,4-dichloro-5-methyl-1H-pyrrol-2-yl)carbonyl]amino-3-azabicyclo[3.1.0]hex-3-yl]4-[2-(methoxycarbonyl)benzyl]-1,3-thiazole-5-carboxylic acid (Compound No. 33),
2-[(1R,5S,6s)-6-{[(3,4-dichloro-5-methyl-1H-pyrrol-2-yl)carbonyl]amino}-3-azabicyclo[3.1.0]hex-3-yl]-4-(phenoxymethyl)-1,3-thiazole-5-carboxylic acid (Compound No. 34),
4-(2-chloro-6-fluorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 35),
2-[(1R,5S,6s)-6-{[(3,4-dichloro-5-methyl-1H-pyrrol-2-yl)carbonyl]amino}-3-azabicyclo[3.1.0]hex-3-yl]-4-(2-phenylethyl)-1,3-thiazole-5-carboxylic acid (Compound No. 36),
2-[(1R,5S,6s)-6-{[(3,4-dichloro-5-methyl-1H-pyrrol-2-yl)carbonyl]amino}-3-azabicyclo[3.1.0]hex-3-yl]-4-(4-fluorobenzyl)-1,3-thiazole-5-carboxylic acid (Compound No. 37),
4-(3-Chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 38),
4-(Cyclopentylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 39),
4-[(Benzyloxy)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 40),
2-[(1R,5S,6s)-6-{[(3,4-dichloro-5-methyl-1H-pyrrol-2-yl)carbonyl]amino}-3-azabicyclo[3.1.0]hex-3-yl]-4-(3-fluorobenzyl)-1,3-thiazole-5-carboxylic acid (Compound No. 41),
4-Cyclohexyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 42),
4-(2-Cyclopentylethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 43),
2-[(1R,5S,6s)-6-[(3,4-dichloro-5-methyl-1H-pyrrol-2-yl)carbonyl]amino}-3-azabicyclo[3.1.0]hex-3-yl]-4-(4-methoxybenzyl)-1,3-thiazole-5-carboxylic acid (Compound No. 44),
2-[(1R,5S,6s)-6-{[(3,4-dichloro-5-methyl-1H-pyrrol-2-yl)carbonyl]amino}-3-azabicyclo[3.1.0]hex-3-yl]-4-(4-fluorophenyl)-1,3-thiazole-5-carboxylic acid (Compound No. 45),
2-[(1R,5S,6s)-6-{[(3,4-dichloro-5-methyl-1H-pyrrol-2-yl)carbonyl]amino}-3-azabicyclo[3.1.0]hex-3-yl]-4-(3-fluorophenyl)-1,3-thiazole-5-carboxyl i c acid (Compound No. 46),
4-(Biphenyl-4-yl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 47),
4-Butyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 48),
2-[(1R,5S,6s)-6-{[(3,4-dichloro-5-methyl-1H-pyrrol-2-yl)carbonyl]amino}-3-azabicyclo[3.1.0]hex-3-yl]-4-(naphthalen-2-yl)-1,3-thiazole-5-carboxylic acid (Compound No. 49),
4-Cyclopentyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 50),
2-[(1R,5S,6s)-6-{[(3,4-dichloro-5-methyl-1H-pyrrol-2-yl)carbonyl]amino}-3-azabicyclo[3.1.0]hex-3-yl]-4-(3,5-dimethoxyphenyl)-1,3-thiazole-5-carboxylic acid (Compound No. 51),
2-[(1R,5S,6s)-6-{[(3,4-dichloro-5-methyl-1H-pyrrol-2-yl)carbonyl]amino}-3-azabicyclo[3.1.0]hex-3-yl]-4-[4-(trifluoromethoxy)phenyl]-1,3-thiazole-5-carboxylic acid (Compound No. 52),
2-[(1R,5S,6s)-6-{[(3,4-dichloro-5-methyl-1H-pyrrol-2-yl)carbonyl]amino}-3-azabicyclo[3.1.0]hex-3-yl]-4-(thiophen-2-yl)-1,3-thiazole-5-carboxylic acid (Compound No. 53),
2-[(1R,5S,6s)-6-{[(3,4-dichloro-5-methyl-1H-pyrrol-2-yl)carbonyl]amino}-3-azabicyclo[3.1.0]hex-3-yl]-4-(3,4-difluorobenzyl)-1,3-thiazole-5-carboxylic acid (Compound No. 54),
4-(1,3-Benzodioxol-5-ylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 55),
4-(2-Chloro-4-fluorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 56),
4-(2-Bromobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 57),
2-[(1R,5S,6s)-6-{[(3,4-dichloro-5-methyl-1H-pyrrol-2-yl)carbonyl]amino}-3-azabicyclo[3.1.0]hex-3-yl]-4-(naphthalen-2-ylmethyl)-1,3-thiazole-5-carboxylic acid (Compound No. 58),
4-(2-Chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 59),
2-[(1R,5S,6s)-6-{[(3,4-dichloro-5-methyl-1H-pyrrol-2-yl)carbonyl]amino}-3-azabicyclo[3.1.0]hex-3-yl]-4-(2-methylbenzyl)-1,3-thiazole-5-carboxylic acid (Compound No. 60),
4-(3-Bromobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 61),
2-[(1R,5S,6s)-6-{[(3,4-dichloro-5-methyl-1H-pyrrol-2-yl)carbonyl]amino}-3-azabicyclo[3.1.0]hex-3-yl]-4-(2-methoxybenzyl)-1,3-thiazole-5-carboxylic acid (Compound No. 62),
2-[(1R,5S,6s)-6-{[(3,4-dichloro-5-methyl-1H-pyrrol-2-yl)carbonyl]amino}-3-azabicyclo[3.1.0]hex-3-yl]-4-(naphthalen-1-ylmethyl)-1,3-thiazole-5-carboxylic acid (Compound No. 63),
4-(2-Chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 64),
4-(3-Chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 65),
4-(4-Bromo-2-methylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 66),
4-(3-Chloro-4-fluorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 67),
4-(4-Chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 68),
2-[(1R,5S,6s)-6-{[(3,4-dichloro-5-methyl-1H-pyrrol-2-yl)carbonyl]amino}-3-azabicyclo[3.1.0]hex-3-yl]-4-[4-(trifluoromethyl)benzyl]-1,3-thiazole-5-carboxylic acid (Compound No. 69),
3,4-Dichloro-5-methyl-N-{(1R,5S,6s)-3-[4-(naphthalen-2-ylmethyl)-1,3-thiazol-2-yl]-3-azabicyclo[3.1.0]hex-6-yl}-1H-pyrrole-2-carboxamide (Compound No. 70),
4-(2,4-Dichlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 71),
4-(4-Butoxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 72),
2-[(1R,5S,6s)-6-{[(3,4-dichloro-5-methyl-1H-pyrrol-2-yl)carbonyl]amino}-3-azabicyclo[3.1.0]hex-3-yl]-4-(4-ethoxybenzyl)-1,3-thiazole-5-carboxylic acid Lithium salt (Compound No. 73),
4-(Biphenyl-4-ylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 74),
4-(Biphenyl-4-yl-methyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Sodium salt (Compound No. 74a)
2-[(1R,5S,6s)-6-{[(3,4-dichloro-5-methyl-1H-pyrrol-2-yl)carbonyl]amino}-3-azabicyclo[3.1.0]hex-3-yl]-4-(5-methylthiophen-2-yl)-1,3-thiazole-5-carboxylic acid Lithium salt (Compound No. 75),
4-[3,5-bis(trifluoromethyl)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 76),
4-[(3′-Chlorobiphenyl-3-yl)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 77),
2-[(1R,5S,6s)-6-{[(3,4-dichloro-5-methyl-1H-pyrrol-2-yl)carbonyl]amino}-3-azabicyclo[3.1.0]hex-3-yl]-4-[(3′,4′-difluorobiphenyl-3-yl)methyl]-1,3-thiazole-5-carboxylic acid Lithium salt (Compound No. 78),
4-(4-Acetylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 79),
4-(4-tert-Butylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 80),
2-[(1R,5S,6s)-6-{[(3,4-dichloro-5-methyl-1H-pyrrol-2-yl)carbonyl]amino}-3-azabicyclo[3.1.0]hex-3-yl]-4-(4-phenoxyphenyl)-1,3-thiazole-5-carboxylic acid Lithium salt (Compound No. 81),
2-[(1R,5S,6s)-6-{[(3,4-dichloro-5-methyl-1H-pyrrol-2-yl)carbonyl]amino}-3-azabicyclo[3.1.0]hex-3-yl]-4-(3-methoxybenzyl)-1,3-thiazole-5-carboxylic acid Lithium salt (Compound No. 82),
4-(4-Carboxyphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 83),
4-(3-Carboxyphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 84),
2-[(1R,5S,6s)-6-{[(3,4-dichloro-5-methyl-1H-pyrrol-2-yl)carbonyl]amino}-3-azabicyclo[3.1.0]hex-3-yl]4-[2-fluoro-5-(trifluoromethyl)benzyl]-1,3-thiazole-5-carboxylic acid Lithium salt (Compound No. 85),
4-(4-Chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 86),
4-(3-Carboxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 87),
2-[(1R,5S,6s)-6-{[(3,4-dichloro-5-methyl-1H-pyrrol-2-yl)carbonyl]amino}-3-azabicyclo[3.1.0]hex-3-yl]-4-(2-ethoxybenzyl)-1,3-thiazole-5-carboxylic acid Lithium salt (Compound No. 88),
4-(4-Carboxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 89),
4-[4-(Acetylamino)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 90),
2-[(1R,5S,6s)-6-{[(3,4-dichloro-5-methyl-1H-pyrrol-2-yl)carbonyl]amino}-3-azabicyclo[3.1.0]hex-3-yl]-4-(hydroxymethyl)-1,3-thiazole-5-carboxylic acid Lithium salt (Compound No. 91),
2-[(1R,5S,6s)-6-{[(3,4-dichloro-5-methyl-1H-pyrrol-2-yl)carbonyl]amino}-3-azabicyclo[3.1.0]hex-3-yl]-4-[4-(ethylcarbamoyl)benzyl]-1,3-thiazole-5-carboxylic acid Lithium salt (Compound No. 92),
4-(4-Carbamoylbenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 93),
2-[(1R,5S,6s)-6-{[(3,4-dichloro-5-methyl-1H-pyrrol-2-yl)carbonyl]amino}-3-azabicyclo[3.1.0]hex-3-yl]-4-[4-(methylcarbamoyl)benzyl]-1,3-thiazole-5-carboxylic acid (Compound No. 94),
2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-oxazole-5-carboxylic acid Lithium salt (Compound No. 95),
2-[(1R,5S,6s)-6-{[(3,4-dichloro-5-methyl-1H-pyrrol-2-yl)carbonyl]amino}-3-azabicyclo[3.1.0]hex-3-yl]-4-[3-(trifluoromethyl)benzyl]-1,3-oxazole-5-carboxylic acid (Compound No. 96),
4-(4-Chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-oxazole-5-carboxylic acid (Compound No. 97),
2-[(1R,5S,6s)-6-{[(3,4-dichloro-5-methyl-1H-pyrrol-2-yl)carbonyl]amino}-3-azabicyclo[3.1.0]hex-3-yl]-4-(4-ethoxybenzyl)-1,3-oxazole-5-carboxylic acid (Compound No. 98),
2-[(1R,5S,6s)-6-{[(3,4-dichloro-5-methyl-1H-pyrrol-2-yl)carbonyl]amino}-3-azabicyclo[3.1.0]hex-3-yl]-4-(pyridin-3-yl)-1,3-thiazole-5-carboxylic acid (Compound No. 99),
2-[(1R,5S,6s)-6-{[(3,4-dichloro-5-methyl-1H-pyrrol-2-yl)carbonyl]amino}-3-azabicyclo[3.1.0]hex-3-yl]-4-(pyridin-4-yl)-1,3-thiazole-5-carboxylic acid (Compound No. 100),
2-[(1R,5S,6s)-6-{[(3,4-dichloro-5-methyl-1H-pyrrol-2-yl)carbonyl]amino}-3-azabicyclo[3.1.0]hex-3-yl]-4-(trifluoromethyl)-1,3-thiazole-5-carboxylic acid (Compound no. 101),
2-[(1R,5S,6s)-6-{[(3,4-dichloro-5-methyl-1H-pyrrol-2-yl)carbonyl]amino}-3-azabicyclo[3.1.0]hex-3-yl]4-[2-(pyridin-4-yl)ethyl]-1,3-thiazole-5-carboxylic acid (Compound no. 102),
N-[3-({2-[(1R,5S,6s)-6-{[(3,4-dichloro-5-methyl-1H-pyrrol-2-yl)carbonyl]amino}-3-azabicyclo[3.1.0]hex-3-yl]-5-(ethoxycarbonyl)-1,3-thiazol-4-yl}methyl)phenyl]glycine (Compound no. 103),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(hydroxymethyl)-1,3-thiazole-5-carboxylate (Compound no. 104),
4-(5-Acetylthiophen-2-yl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 105),
4-({2-[(1R,5S,6s)-6-{[(3,4-dichloro-5-methyl-1H-pyrrol-2-yl)carbonyl]amino}-3-azabicyclo[3.1.0]hex-3-yl]-5-(ethoxycarbonyl)-1,3-thiazol-4-yl}methyl)benzoic acid Lithium salt (Compound no. 106),
4-[3-(Acetylamino)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 107),
2-[(1R,5S,6s)-6-{[(3,4-dichloro-5-methyl-1H-pyrrol-2-yl)carbonyl]amino}-3-azabicyclo[3.1.0]hex-3-yl]-4-[3-(trifluoromethyl)benzyl]-1,3-thiazole-5-carboxylic acid Lithium salt (Compound no. 108),
2-[(1R,5S,6 s)-6-{[(3,4-dichloro-5-methyl-1H-pyrrol-2-yl)carbonyl]amino}-3-azabicyclo[3.1.0]hex-3-yl]-4-{3-[(ethylcarbamoyl)amino]benzyl}-1,3-thiazole-5-carboxylic acid Lithium salt (Compound no. 109),
4-(2-Carboxybenzyl)-2-{[(1R,5S,6s)-6-1 [(3,4-dichloro-5-methyl-1H-pyrrol-2-yl)carbonyl]amino}-3-azabicyclo[3.1.0]hex-3-yl]-1,3-thiazole-5-carboxylic acid (Compound no. 110),
4-(2-Carboxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 111),
2-({5-(tert-butoxycarbonyl)-2-[(1R,5S,6s)-6-{[(3,4-dichloro-5-methyl-1H-pyrrol-2-yl)carbonyl]amino}-3-azabicyclo[3.1.0]hex-3-yl}-1,3-thiazol-4-yl]methyl)benzoic acid (Compound no. 112),
2-[(1R,5S,6s)-6-{[(3,4-dichloro-5-methyl-1H-pyrrol-2-yl)carbonyl]amino}-3-azabicyclo[3.1.0]hex-3-yl]-4-[2-(ethylamino)-2-oxoethyl]-1,3-thiazole-5-carboxylic acid (Compound no. 113),
4-(2-{4-[(tert-Butoxycarbonyl)amino]piperidin-1-yl]-2-oxoethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 114),
4-[(3′-Carboxybiphenyl-3-yl)methyl]-2-{[(1R,5S,6 s)-6-[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 115),
2-[(1R,5S,6s)-6-{[(3,4-dichloro-5-methyl-1H-pyrrol-2-yl)carbonyl]amino}-3-azabicyclo[3.1.0]hex-3-yl]-4-[3-(6-fluoropyridin-3-yl)benzyl]-1,3-thiazole-5-carboxylic acid (Compound no. 116),
2-[(1R,5S,6s)-6-{[(3,4-dichloro-5-methyl-1H-pyrrol-2-yl)carbonyl]amino}-3-azabicyclo[3.1.0]hex-3-yl]-4-{4-[(4-hydroxypiperidin-1-yl)carbonyl]benzyl}-1,3-thiazole-5-carboxylic acid (Compound no. 117),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-{4-[(ethylcarbamoyl)amino]benzyl}-1,3-thiazole-5-carboxylate (Compound no. 118),
4-[2-(Cyclopropylamino)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 119),
4-[2-(Cyclopentylamino)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 120),
4-{2-[(Carboxymethylamino)-2-oxoethyl}-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 121),
N-({2-[(1R,5S,6s)-6-{[(3,4-dichloro-5-methyl-1H-pyrrol-2-yl)carbonyl]amino}-3-azabicyclo[3.1.0]hex-3-yl]-5-(methoxycarbonyl)-1,3-thiazol-4-yl]acetypglycine Lithium salt (Compound no. 122),
4-[2-(Cyclobutylamino)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 123),
2-[(1R,5S,6s)-6-{[(3,4-dichloro-5-methyl-1H-pyrrol-2-yl)carbonyl]amino}-3-azabicyclo[3.1.0]hex-3-yl]-4-(2-oxo-2-{[3-(trifluoromethyl)phenyl]amino ethyl)-1,3-thiazole-5-carboxylic acid Lithium salt (Compound no. 124),
4-{2-[(3-Chlorophenyl)amino]-2-oxoethyl}-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 125),
2-[(1R,5S,6s)-6-{[(3,4-dichloro-5-methyl-1H-pyrrol-2-yl)carbonyl]amino}-3-azabicyclo[3.1.0]hex-3-yl]4-[2-(4-hydroxypiperi din-1-yl)-2-oxoethyl]-1,3-thiazole-5-carboxylic acid (Compound no. 126),
2-[(1R,5S,6s)-6-{[(3,4-dichloro-5-methyl-1H-pyrrol-2-yl)carbonyl]amino}-3-azabicyclo[3.1.0]hex-3-yl]4-[2-(morpholin-4-yl)-2-oxoethyl]-1,3-thiazole-5-carboxylic acid (Compound no. 127),
2-[(1R,5S,6s)-6-{[(3,4-dichloro-5-methyl-1H-pyrrol-2-yl)carbonyl]amino}-3-azabicyclo[3.1.0]hex-3-yl]4-[2-(methoxyamino)-2-oxoethyl]-1,3-thiazole-5-carboxylic acid (Compound no. 128),
2-[(1R,5S,6s)-6-{[(3,4-dichloro-5-methyl-1H-pyrrol-2-yl)carbonyl]amino}-3-azabicyclo[3.1.0]hex-3-yl]4-[2-oxo-2-(pyridin-3-ylamino)ethyl]-1,3-thiazole-5-carboxylic acid (Compound no. 129),
4-[2-(Azetidin-1-yl)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 130),
2-[(1R,5S,6s)-6-{[(3,4-dichloro-5-methyl-1H-pyrrol-2-yl)carbonyl]amino}-3-azabicyclo[3.1.0]hex-3-yl]4-[2-(4-methylpiperazin-1-yl)-2-oxoethyl]-1,3-thiazole-5-carboxylic acid (Compound no. 131),
4-{2-[4-(tert-Butoxycarbonyl)piperazin-1-yl]-2-oxoethyl]-2-[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 132),
2-[(1R,5S,6s)-6-{[(3,4-dichloro-5-methyl-1H-pyrrol-2-yl)carbonyl]amino}-3-azabicyclo[3.1.0]hex-3-yl]-4-(2-oxo-2-{[4-(4H-1,2,4-triazol-4-yl)phenyl]amino}ethyl)-1,3-thiazole-5-carboxylic acid (Compound no. 133),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(4-methoxybenzyl)amino]-2-oxoethyl}-1,3-thiazole-5-carboxylic acid (Compound no. 134),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(4-methyl-1,3-thiazol-2-yl)amino]-2-oxoethyl}-1,3-thiazole-5-carboxylic acid (Compound no. 135),
2-[(1R,5S,6s)-6-{[(3,4-dichloro-5-methyl-1H-pyrrol-2-yl)carbonyl]amino}-3-azabicyclo[3.1.0]hex-3-yl]-4-(2-oxo-2-{[4-(trifluoromethyl)benzyl]amino]ethyl)-1,3-thiazole-5-carboxylic acid (Compound no. 136),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(3-fluorophenyl)amino]-2-oxoethyl)-1,3-thiazole-5-carboxylic acid (Compound no. 137),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(4-ethylphenyl)amino]-2-oxoethyl}-1,3-thiazole-5-carboxylic acid (Compound no. 138),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(3-methylbenzyl)amino]-2-oxoethyl}-1,3-thiazole-5-carboxylic acid (Compound no. 139),
2-[(1R,5S,6s)-6-{[(3,4-dichloro-5-methyl-1H-pyrrol-2-yl)carbonyl]amino}-3-azabicyclo[3.1.0]hex-3-yl]4-[2-oxo-2-(piperidin-1-yl)ethyl]-1,3-thiazole-5-carboxylic acid (Compound no. 140),
4-(2-{3-[(tert-Butoxycarbonyl)amino]pyrrolidin-1-yl)-2-oxo ethyl)-2-[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 141),
4-[2-(3-Aminopyrrolidin-1-yl)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 142),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(1-ethyl-1H-pyrazol-5-yl)amino]-2-oxoethyl}-1,3-thiazole-5-carboxylic acid (Compound no. 143),
4-[2-(4-Aminopiperidin-1-yl)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxyli c acid (Compound no. 144),
2-[(1R,5S,6s)-6-{[(3,4-dichloro-5-methyl-1H-pyrrol-2-yl)carbonyl]amino}-3-azabicyclo[3.1.0]hex-3-yl]-4-[2-oxo-2-(piperazin-1-yl)ethyl]-1,3-thiazole-5-carboxylic acid (Compound no. 145),
2-[(1R,5S,6s)-6-{[(3,4-dichloro-5-methyl-1H-pyrrol-2-yl)carbonyl]amino}-3-azabicyclo[3.1.0]hex-3-yl]4-[2-oxo-2-(1,3-thiazol-2-yl)ethyl]-1,3-thiazole-5-carboxylic acid (Compound no. 146),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(3-methyl-1,2-oxazol-5-yl)amino]-2-oxoethyl}-1,3-thiazole-5-carboxylic acid (Compound no. 147),
4-[2-(1H-Benzimidazol-2-ylamino)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxyli c acid (Compound no. 148),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(2-hydroxyethyl)amino]-2-oxoethyl}-1,3-thiazole-5-carboxylic acid (Compound no. 149),
4-{2-[(3-Chlorobenzyl)amino]-2-oxoethyl}-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 150),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(1-hydroxy-2-methylpropan-2-yl)amino]-2-oxoethyl}-1,3-thiazole-5-carboxylic acid (Compound no. 151),
4-{2-[(4-Carboxyphenyl)amino]-2-oxoethyl}-2-[(1R,5S,6s)-6-[(3,4-dichloro-5-methyl-1H-pyrrol-2-yl)carbonyl]aminol-3-azabicyclo[3.1.0]hex-3-yl]-1,3-thiazole-5-carboxylic acid (Compound no. 152),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxylate (Compound no. 153),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 154),
Methyl 2-chloro-6-[(1R,5S,6s)-6-{[(3,4-dichloro-5-methyl-1H-pyrrol-2-yl)carbonyl]amino}-3-azabicyclo[3.1.0]hex-3-yl]pyridine-4-carboxylate (Compound no. 155),
Methyl 6-[(1R,5S,6s)-6-{[(3,4-dichloro-5-methyl-1H-pyrrol-2-yl)carbonyl]amino}-3-azabicyclo[3.1.0]hex-3-yl]pyridine-3-carboxylate (Compound no. 156),
Methyl 6-[(1R,5S,6s)-6-{[(3,4-dichloro-5-methyl-1H-pyrrol-2-yl)carbonyl]amino}-3-azabicyclo[3.1.0]hex-3-yl]-2-(morpholin-4-yl)pyrimidine-4-carboxylate (Compound no. 157),
Methyl 2-[(cyclopropylmethyl)amino]-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylate (Compound no. 158),
Methyl 2-{4-[(tert-butoxycarbonyl)amino]piperidin-1-yl]-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylate (Compound no. 159),
tert-Butyl 2-[(1R,5S,6s)-6-{[(3,4-dichloro-5-methyl-1H-pyrrol-2-yl)carbonyl]amino}-3-azabicyclo[3.1.0]hex-3-yl]-4-[2-(methoxycarbonyl)benzyl]-1,3-thiazole-5-carboxylate (Compound no. 160),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(methoxymethyl)-1,3-thiazole-5-carboxylate (Compound no. 161),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(phenoxymethyl)-1,3-thiazole-5-carboxylate (Compound no. 162),
Ethyl 4-(cyclopentylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 163),
Ethyl 4-[(benzyloxy)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 164),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(3-fluorobenzyl)-1,3-thiazole-5-carboxylate (Compound no. 165),
Ethyl 4-(2-cyclopentylethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 166),
Ethyl 4-cyclohexyl-2-[(1R,5S,6s)-6-1 [(3,4-dichloro-5-methyl-1H-pyrrol-2-yl)carbonyl]amino-3-azabicyclo[3.1.0]hex-3-yl]-1,3-thiazole-5-carboxylate (Compound no. 167),
Ethyl 4-(2-chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 168),
Ethyl 4-(4-bromo-2-methylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 169),
Ethyl 2-[(1R,5S,6s)-6-{1 [(3,4-dichloro-5-methyl-1H-pyrrol-2-yl)carbonyl]amino}-3-azabicyclo[3.1.0]hex-3-yl]-4-[3-(6-fluoropyridin-3-yl)benzyl]-1,3-thiazole-5-carboxylate (Compound no. 170),
Ethyl 4-(2,4-dichlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 171),
Ethyl 4-(4-butoxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 172),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(3-methoxybenzyl)-1,3-thiazole-5-carboxylate (Compound no. 173),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(4-ethoxybenzyl)-1,3-thiazole-5-carboxylate (Compound no. 174),
Ethyl 4-(biphenyl-4-ylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 175),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(2,5-dimethoxybenzyl)-1,3-thiazole-5-carboxylate (Compound no. 176),
tert-Butyl 2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxylate (Compound no. 177),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(pyridin-4-yl)-1,3-thiazole-5-carboxylate (Compound no. 178) and
{2-[(1R,5S,6s)-6-{[(3,4-dichloro-5-methyl-1H-pyrrol-2-yl)carbonyl]amino}-3-azabicyclo[3.1.0]hex-3-yl]-5-(methoxycarbonyl)-1,3-thiazol-4-yl]acetic acid (Compound no. 179).

In yet another aspect, provided herein are pharmaceutical compositions comprising therapeutically effective amounts of one or more compounds described herein together with one or more pharmaceutically acceptable carriers, excipients or diluents.

In another aspect, provided herein are methods for treating or preventing conditions caused by or contributed to by bacterial infections comprising administering to a mammal in need thereof therapeutically effective amount of one or more compounds of Formula I described herein.

The methods may include one or more of the following embodiments. For example, the condition can be selected from community acquired pneumonia, upper or lower respiratory tract infections, complicated skin and skin structure infections (cSSSI), uncomplicated skin and soft structure infections, hospital acquired (nosocomial) infections, urinary tract infections, intra-abdominal infections, enterococci infections, bacteraemia infections with known or suspected endocarditis, nosocomial bone or joint infections, acne vulgaris, mastitis, catheter infection, foreign body, prosthesis infections or peptic ulcer disease.

In another embodiment, the bacterial infections can be caused by gram positive, gram negative or anaerobic bacteria.

In another embodiment, the gram positive, gram negative or anaerobic bacteria can be selected from Staphylococci (S. aurues including MRSA, S. epidermidis including MRSE, CoNS, etc.) Streptococci (S. pneumoniae, S. pyogens, S. viridans, S. agalactiae, etc.) Enterococci (E. faecalis, E. faecium, etc.), Haemophilus spp., Moraxalla spp., Chlamydia spp., Mycoplasma spp., Legionella spp., Mycobacterium tuberculosis (including MDR and XDR strains), Helicobacter pylori, Clostridium spp., P. acne., Bacteroides spp., Corynebacterium, Bacillus spp., Enterobactericeae (E. coli, Klebsiella spp., Proteus spp., etc) and Pseudomonas spp.

In another embodiment, the bacterium is cocci.

In another embodiment, the cocci are drug resistant.

In another embodiment, the drug resistant cocci are selected from methicillin resistant Staphylococcus aureus (MRSA), vancomycin resistant S. aureus (VRSA), methicillin resistant Staphylococcus epidermidis (MRSE), Streptococcus pyogenes (erm, mef, telithromycin resistance), Enterococcus faecalis and faecium (vancomycin and telithromycin resistance), penicillin resistant Streptococcus pneumoniae (PRSP), and multi-drug resistant Streptococcus pneumoniae.

In another aspect, provided herein are methods for treating, preventing or inhibiting nosocomial and/or community acquired bacterial infection or a associated disease, disorder or infection thereof, comprising administering to a mammal in need thereof, a therapeutically effective amount of one or more compounds of Formula I or its pharmaceutically acceptable salts, esters, polymorphs, pharmaceutically acceptable solvates, co-crystals, enantiomers, diastereomers, N-oxides, prodrugs or metabolites thereof, in combination with one or more therapeutic agents selected from other antibacterial compounds, for example, protein synthesis inhibitors (linezolid, telithromycin, tigecycline, etc,) aminoglycosides (gentamycin, kanamycin, etc), cell wall synthesis inhibitors (glycopeptides, for example, vancomycin, teicoplanin, telavancin, bleomycin, etc, beta-lactams, for example, penicillin, methicillin, etc.), RNA and DNA synthesis inhibitors (quinolones such as nalidixic acid, oxolinic acid etc, fluoquinolones such as ciprofloxacin, levofloxacin, moxifloxacin, etc.) fatty acid synthesis inhibitors and its derivatives and other therapeutic agents, which can be used to treat, prevent or inhibit nosocomial and community acquired bacterial infection or a associated disease, disorder or infection thereof.

In another aspect, provided herein are methods for treating or preventing acne vulgaris and inflammatory conditions thereof comprising administering to a mammal in need thereof therapeutically effective amounts of one or more compounds of Formula I in combination with one or more therapeutic agents selected from alcohol, benzoyl peroxide, clindamycin, tretinoin, vitamin E, vitamin A and its derivatives, tetracycline, isotretinoin, vitamin C, vitamin D, chaparral, dandelion root, licoric root, Echinacea, kelp, cayenine, sassafras, elder flowers, pantothenic acid, para amino benzoic acid, biotin, cholin, inositol, folic acid, calcium, magnesium, potassium, vitamin B₆, zinc, carotenoid, azelaic acid, and other therapeutic agents, which can be used to treat acne or condition the skin.

In another aspect, provided herein is the use of a pharmaceutical composition of the combination of the compounds of the said invention with various other therapeutic agents as described above in the manufacture of a medicament for treating, preventing or inhibiting nosocomial or community acquired bacterial infection or any associated disease, disorder or infection thereof.

### DEFINITIONS

The following definitions apply to terms as used herein:

The term “alkenyl”, unless and otherwise specified, refers to a branched or unbranched unsaturated hydrocarbon group containing at least one double bond with cis or trans geometry and preferably having 2 to 20 carbon atoms. It may further be substituted with one or more of the substituents selected from the group consisting of alkyl, alkynyl, cycloalkyl, cycloalkenyl, aryl, heterocyclyl, heteroaryl, —OR_{λ}, —SR_{λ}, —C(═O)—R_{λ}, —C(═S)—R_{λ}, azido, cyano, halo, —C(═O)OR_{λ}, nitro, —NHC(═O)R_{λ}, —NR_{λ}R_{π}, —C(═O)NR_{λ}R_{π}, —NHC(═O)NR_{λ}R_{π}, —O—C(═O)NR_{λ}R_{π} [wherein R_{λ} and R_{π} are defined as above; R_{λ} and R_{π} may together form a ring], —NHSO₂R_{ψ} and —SO₂R_{ψ} (wherein R_{ψ} is defined as above).

The term “alkynyl”, unless and otherwise specified, refers to a branched or unbranched unsaturated hydrocarbon group containing at least one triple bond and preferably having 2 to 20 carbon atoms. It may further be substituted with one or more substituents selected form the group consisting of alkyl, alkenyl, cycloalkyl, cycloalkenyl, aryl, heterocyclyl, heteroaryl, —OR_{λ}, —C(═O)—R_{λ}, C(═S)—R_{λ}, —O—C(═O)—R_{λ}, azido, cyano, halo, —C(═O)OR_{λ}, nitro, —NHC(═O)R_{λ}, —NR_{λ}R_{π}, —C(═O)NR_{λ}R_{π}, —NHC(═O)NR_{λ}R_{π}, —O—C(═O)NR_{λ},R_{π} [wherein R_{λ} and R_{π} are defined as above; R_{λ} and R_{π} may together form a ring], —NHSO₂R_{ψ} and —SO₂R_{ψ} (wherein R_{ψ} is the same as defined above).

The term “alkylene,” as used herein, refers to a diradical branched or unbranched saturated hydrocarbon chain having from 1 to 6 carbon atoms and one or more hydrogen can optionally be substituted with alkyl, hydroxy, halogen or oximes. This term can be exemplified by groups such as methylene, ethylene, propylene isomers (e.g., —CH₂CH₂CH₂, —CH(CH₃)₂, and —CH(CH₃)CH2) and the like. Alkylene may further be substituted with one or more substituents such as alkyl, alkenyl, alkynyl, alkoxy, cycloalkyl, acyl, acylamino, acyloxy, alkoxycarbonylamino, azido, cyano, halo, hydroxy, thio, carboxy, arylthio, thiol, alkylthio, aryloxy, heteroaryloxy, aminosulfonyl, —OR_{λ}, —C(═O)R_{λ}, —C(═S)R_{λ}, —OC(═O)R_{λ}, —COOR_{ψ}, —NHC(═O)R_{λ}, —NR_{λ}R_{π}, —C(═O)NR_{λ}R_{π}, —NHC(═O)NR_{λ}R_{π}, —C(═O)heteroaryl, C(═O)heterocyclyl, —O—C(═O)NR_{λ}R_{π}, nitro, —S(O)ₘR_{λ}, (wherein R_{λ}, R_{π}, m and R_{ψ} are the same as defined earlier). Unless otherwise constrained by the definition, all substituents may be further substituted by 1-3 substituents chosen from alkyl, alkenyl, alkynyl, carboxy, —COOR_{ψ}, —C(═O)NR_{λ}R_{π}, —OC(═O)NR_{λ}R_{π}, —NHC(═O)NR_{λ}R_{π}, hydroxy, alkoxy, halogen, CF₃, cyano, and —S(O)ₘR_{ψ} (wherein R_{λ}, R_{π}, m and R_{ψ} are the same as defined earlier). Alkylene can also be optionally interrupted by 1-5 atoms of groups independently chosen from oxygen, sulfur and —NR_{α} (wherein R_{α} is the same as defined earlier). Unless otherwise constrained by the definition, all substituents may be further substituted by 1-3 substituents selected from hydrogen, alkyl, cycloalkyl, alkenyl, alkynyl, aryl, acyl, aralkyl, alkoxy, hydroxy, carboxy, —C(═O)OR_{ψ}, halogen, CF₃, cyano, —NR_{λ}R_{π}, —S(O)ₘR_{ψ}, —C(═O)NR_{λ}R_{π}, —OC(═O)NR_{λ}R_{π}, —CONH—, —C═O or —C═NOH (wherein R_{λ}, R_{π}, m and R_{ψ} are the same as defined earlier).

The term “alkoxy” denotes the group O-alkyl, wherein alkyl is the same as defined above.

The term “aryloxy” denotes the group O-aryl, wherein aryl is as defined above.

The term “heteroaryloxy” denotes the group O-heteroaryl, wherein heteroaryl is as defined above.

The term “heterocyclyloxy” denotes the group O-heterocyclyl, wherein heterocyclyl is as defined above.

The term “aralkyl” or “arylalkyl” refers to alkyl-aryl linked through an alkyl portion (wherein alkyl is as defined above) and the alkyl portion contains 1-8 carbon atoms and aryl is as defined below.

The term “heteroarylalkyl” refers to alkyl-heteroaryl group linked through alkyl portion, wherein the alkyl and heteroaryl are as defined earlier.

The term “heterocyclylalkyl” refers to alkyl-heterocyclyl group linked through alkyl portion, wherein the alkyl and heterocyclyl are as defined earlier.

The term “aralkyloxy” or “arylalkyloxy” refers to the group O-alkyl-aryl, wherein alkyl and aryl is as defined above

The term “heteroarylalkyloxy” refers to O-alkyl-heteroaryl group, wherein the alkyl and heteroaryl are as defined earlier.

The term “heterocyclylalkyloxy” refers to O-alkyl-heterocyclyl group, wherein the alkyl and heterocyclyl are as defined earlier.

The term “alkyamino” refers to alkyl-amino group linked through alkyl portion, wherein the alkyl and amino are as defined earlier.

The term “alkylcarbonyl” or “acyl” refers to —C(═O)R_{¢¢} wherein R_{¢¢} is selected from hydrogen, alkyl, cycloalkyl, aryl, aralkyl, heteroaryl, heterocyclyl, heteroarylalkyl or heterocyclylalkyl.

The term “alkoxycarbonyl” refers to —C(═O)OR_{¢¢}, wherein R_{¢¢} is as defined earlier

The term “alkylcarboxy” refers to —O—C(═O)R_{¢¢}, wherein R_{¢¢} is as defined earlier.

The term “amine or amino” unless otherwise specified, refers to —NH₂. “Substituted amino” unless otherwise specified, refers to a group —N(R_{z})₂ wherein each R_{z} is independently selected from the group hydrogen provided that both R_{z} groups are not hydrogen (defined as “amino”), alkyl, alkenyl, alkynyl, aralkyl, cycloalkyl, aryl, heteroaryl, heterocyclyl, heterocyclylalkyl, heteroarylalkyl, acyl, S(O)ₘR_{ψ} (wherein m and R_{ψ} are the same as defined above), —C(═Rᵥ)NR_{λ}R_{y} (wherein Rᵥ is O or S & R_{λ} and R_{y} are the same as defined earlier) or NHC(═Rᵥ)NR_{y}R_{λ} (wherein Rᵥ, R_{y} and R_{λ} are the same as defined earlier). Unless otherwise constrained by the definition, all amino substituents may optionally be further substituted by 1-3 substituents chosen from alkyl, aralkyl, cycloalkyl, aryl, heteroaryl, heterocyclyl, carboxy, —COOR_{ψ} (wherein R_{ψ} is the same as defined earlier), hydroxy, alkoxy, halogen, CF₃, cyano, —C(═Rv)NR_{λ}R_{y} (wherein Rᵥ is the same as defined earlier), —O(C═O)NR_{λ}R_{y}, —OC(═Rᵥ)NR_{λ}R_{y} (wherein R_{λ}, R_{y} and Rᵥ are the same as defined earlier), —S(O)ₘR_{ψ} (wherein R_{ψ} and m are the same as defined above).

The term “carboxy,” as defined herein, refers to —C(═O)OR_{λ} (wherein R_{λ} is defined earlier).

The term “halogen or halo” refers to fluorine, chlorine, bromine or iodine.

The term “haloalkyl” refers to alkyl of which one or more hydrogen(s) is/are replaced by halogen.

The term “oxo” refers to —C(═O).

The term “protecting group” is used herein to refer to known moieties which have the desirable property of preventing specific chemical reaction at a site on the molecule undergoing chemical modification intended to be left unaffected by the particular chemical modification. Also the term “protecting group”, unless or otherwise specified, may be used with groups such as hydroxy, amino, and carboxy. The examples of such groups are found in T. W. Greene and P. G. M. Wuts, “Protective groups in organic synthesis”, 3^{rd} ed., John Wiley and Sons Inc., New York, 1999, which is incorporated herein by reference.

The term “pharmaceutically acceptable salts” refers to the inorganic and organic base or acid addition salts of compounds of present invention. These salts can be prepared in situ during the final isolation and purification of the compounds or by separately reacting the purified compound in its free form with a suitable organic or inorganic base or acid and isolating the salt thus obtained. Representative salts include, but not limited to, trifluoroacetate, hydrochloride, acetate, fumarate, phosphate, tosylate, hydrobromide, sulfate, bisulfate, nitrate, oxalate, valerate, oleate, palmitate, stearate, laurate, borate, benzoate, lactate, citrate, maleate, succinate, tartrate, naphthylate, mesylate, glucoheptonate, lactobionate, laurylsulfonate and the like. Where the compounds carry acidic moiety, the salts derived from inorganic bases include, but not limited to, lithium, sodium, potassium, calcium, magnesium, zinc, aluminium as well as non-toxic ammonium, quaternary ammonium and amine cations, including, but not limited to ammonium, tetramethylammonium, tetraethylammonium, methylamine, triethylamine, ethylamine, diethylamine, and the like. The salts derived from organic bases include, but not limited to, salts of natural or synthetic amino acids, betaine, caffeine, 2-diethylaminoethanol, N-ethylmorpholine, glucosamine, dibenzylethylene-diamine, chloroprocaine, choline, diethanolamine, ethylenediamine, piperazine, procaine, purine, tromethamine and the like. The free base form may be regenerated by contacting the salt form with a base. While the free base form may differ from the salt form in terms of physical properties, such as solubility, the salts are equivalent to their respective free bases for the purposes of the present invention.

The term “pharmaceutically acceptable solvates” refers to solvates with water (i.e., hydrates) or pharmaceutically acceptable solvents, for example solvates with ethanol and the like. Such solvates are also encompassed within the scope of the disclosure. Furthermore, some of the crystalline forms for compounds described herein may exist as polymorphs and as such are intended to be included in the scope of the disclosure.

The present invention within its scope also includes ‘prodrugs’ of these agents. In general, such prodrugs will be functional derivatives of these compounds, which are readily convertible in vivo into the active drugs. Conventional procedure for the selection and preparation of suitable prodrug derivatives are described, for example, in “Targeted prodrug design to optimize drug delivery”, AAPS PharmSci. 2000, 2(1), E6.

The term “pharmaceutically acceptable carriers” is intended to include non-toxic, inert solid, semi-solid or liquid filler, diluent, encapsulating material or formulation auxiliary of any type.

The term “co-crystals” defines the crystalline phase wherein at least two components of the crystal interact by hydrogen bonding and possibly by other non-covalent interactions rather than by ion pairing.

The term “polymorphs” refers to all crystalline forms and amorphous forms of the compounds described herein. In addition, some of the compounds described herein may form solvates with water or common organic solvents. Such solvates are also encompassed within the scope of this invention.

The term “Community-acquired infections” relates to the infections acquired from the community in the patients, who had not recently been in a health care facility or been in contact with someone who had been recently in a health care facility. Community-acquired respiratory tract infection (CARTI) is a common cause of acute illness in adults and includes, community acquired pneumonia, mild to severe upper and lower respiratory tract infections, acute bronchitis, chronic obstructive pulmonary disease.

The term “Hospital-acquired infections (nosocomial infections)” also known as health-care associated infections relates to the infections acquired by patients from the surrounding bacterial pool in hospital setup. Patients contract these infections from pathogens on the hands of medical personnel, invasive procedures (e.g., intubations and extended ventilation, indwelling vascular lines, urine catheterization), or contaminated air-conditioning systems, contaminated water systems. Most serious hospital acquired infections include ventilator-associated pneumonia (VAP), lower respiratory infection, catheter related infection, foreign body, prosthesis infections or peptic ulcer disease, skin, soft tissue, and surgical-site infections.

In another aspect, the compounds disclosed herein may be prepared by the following reaction sequences as depicted in Schemes I-VII.

The compound of Formula VIII can be prepared by following synthetic route as described in Scheme I.

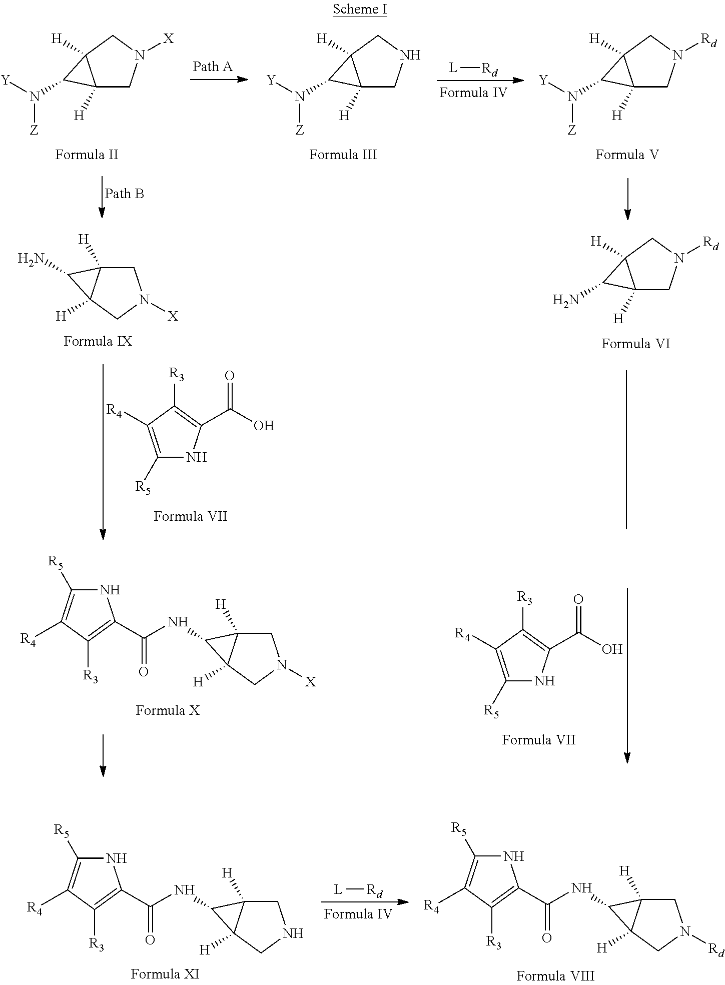

The compound of Formula II can react through two pathways.
Path A: The compound of Formula II (wherein y is H or an amine-protecting group and Z is an amine-protecting group, for example, t-butoxycarbonyl (t-BOC), 9-fluorenylmethoxycarbonyl (Fmoc), phthalimide, trityl, allyloxycarbonyl, trifluoroacetamide, tosyl, benzyl, benzyloxycarbonyl or pyridine-2-sulfonyl and X is an amine protecting group, for example, benzyl, benzyloxycarbonyl, trifluoroacetyl, allyloxycarbonyl, t-butoxycarbonyl (t-BOC) or pyridine-2-sulfonyl) can be N-deprotected to give a compound of Formula III. The protecting groups used in compounds of Formula II can be such that selective removal of one is possible for example X can be benzyl and Y can be t-Boc. A compound of Formula III can react with a compound of Formula IV (wherein R_{d} is (un)substituted aryl, heteroaryl and L is a leaving group, for example, chloro, bromo, tosyl etc.) to give a compound of Formula V. The compound of Formula V can again be N-deprotected to give a compound of Formula VI. The compound of Formula VI can be coupled with a compound of Formula VII (wherein R₃, R₄ and R₅ are as defined earlier) to give a compound of Formula VIII.
Path B: The compound of Formula II can be deprotected to give a compound of Formula IX. The compound of Formula IX can then be coupled with a compound of Formula VII to give a compound of Formula X. The compound of Formula X can be N-deprotected to give a compound of Formula XI, which can then be reacted with a compound of Formula IV to give a compound of Formula VIII.

N-deprotection of compound of Formula II to give a compound of Formula III (Path A) can be carried out by transfer catalytic hydrogenation using palladium-carbon in the presence of a hydrogen donor, for example, ammonium formate, cyclohexene, hydrazine hydrate, or 1,4-cyclohexadiene in one or more solvent, for example, methanol, ethanol, isopropanol, n-propanol or formic acid.

Alternatively, the reaction can also be carried out by hydrogenation to give a compound of Formula III in the presence of one or more reducing agent, for example, palladium-carbon/hydrogen, raney nickel/hydrogen, platinum/hydrogen or mixture thereof in one or more alcohols, for example, methanol, ethanol, propanol, isopropanol or mixtures thereof.

The reaction of compound of Formula III with a compound of Formula IV to give a compound of Formula V can be carried out using a base, for example, potassium carbonate, cesium carbonate, lithium hydroxide, N,N-diisopropyl ethyl amine, N-methylmorpholine or triethylamine in the presence of one or more solvent, for example, N,N-dimethylformamide, dioxane, dimethylsulphoxide or tetrahydrofuran.

Alternatively, the coupling of a compound of Formula III with a compound of Formula IV to give a compound of Formula V can also be carried out with 2,2′-bis(diphenylphosphino)-1,1′-binaphthyl (BINAP), XPhos, XantPhos in the presence of a catalyst, for example, Tris(dibenzylideneacetone)dipalladium palladium(II)acetate, [1,1′-Bis(diphenylphosphino)ferrocene]palladium(II)chloride, Bis(triphenyl-phosphine)palladium(II)chloride, in one or more bases, for example, cesium carbonate, potassium carbonate, potassium phosphate, sodium-t-butoxide, 1,8-diazabicyclo[5.4.0]undec-7-ene, 7-methyl-1,5,7-triazabicyclo[4.4.0]dec-5-ene in one or more solvents, for example, toluene, dioxane or dimethoxyethane.

The N-deprotection of compound of Formula V to give a compound of Formula VI (wherein the protecting group is an acid labile group, for example, t-butyl carbamate) can be carried out in the presence of an acid, for example, hydrochloric acid, hydrobromic acid, hydroiodic acid, trifluoroacetic acid or p-toluene sulfonic acid in one or more solvents, for example, diethyl ether, dioxane, dichloromethane, acetonitrile, methanol, ethanol, propanol, isopropanol, butanol or water.

The coupling of the compound of Formula VI with a compound of Formula VII to give a compound of Formula VIII can be carried out in one or more solvent, for example, dimethylformamide, tetrahydrofuran or dioxane using a coupling agent, for example, 1-ethyl-3-(3′-dimethylaminopropyl)carbodiimide hydrochloride (EDCI), 1,3-dicyclohexyl-carbodiimide (DCC), N-[(dimethylamino)-1H-1,2,3-triazolo[4,5-b]pyridylmethylene]-N-methylmethanaminium hexafluorophosphate N-oxide (HATU) or benzotriazol-1-yl-N-oxy-tris(pyrrolidino)phosphonium hexafluorophosphate (PyBOP) and, optionally, a catalyst, for example, 1-hydroxybenzotriazole (HOBO, 3-hydroxy-3,4-dihydro-4-oxo-1,2,3-benzotriazine (HODhbt) or 7-aza-1-hydroxybenzotriazole (HOAt) and, optionally, with a base, for example, N-methyl-morpholine (NMM), N,N-dimethylaminopyridine (DMAP), triethylamine (TEA) or N,N-diisopropylethylamine (DTEA).

The N-deprotection of compound of Formula II (Path B) (wherein the protecting group is an acid labile group, for example, t-butyl carbamate) to give a compound of Formula IX can be carried out in the similar way as the deprotection of compound of Formula V to give a compound of Formula VI.

The coupling of compound of Formula IX with a compound of Formula VII to give a compound of Formula X can be carried out in a similar way as the coupling of compound of Formula VI with a compound of Formula VII to give a compound of Formula VIII.

The N-deprotection of compound of Formula X to give a compound of Formula XI can be carried out in a base, for example, potassium carbonate, sodium carbonate, caesium carbonate or lithium hydroxide in one or more solvents, for example, methanol, water, ethanol, isopropanol or n-propanol.

The compound of Formula XI can be reacted with compound of Formula IV to give a compound of Formula VIII can be carried out with carbonates, for example, potassium carbonate, sodium carbonate or cesium carbonate, in one or more solvents, for example, dimethylformamide, tetrahydrofuran, dimethylsulfoxide, dioxane or acetonitrile.

Alternatively, the reaction can be carried out using methods similar to those used for coupling of a compound of Formula III with a compound of Formula IV.

The compound of Formula XII can be prepared by following the Scheme II as follows

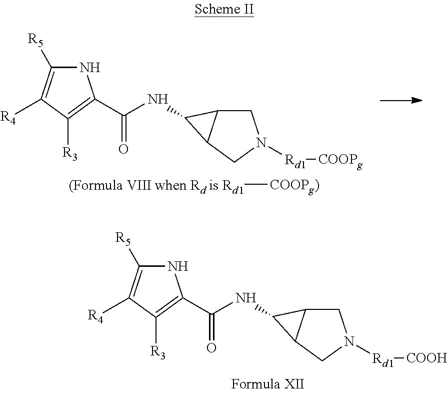

Compound of Formula VIII (wherein R_{d} is R_{d1}—COOP_{g} and R_{d1} is aryl or heteroaryl, P_{g} is an alkyl group, for example, methyl, ethyl or t-butyl) can be hydrolyzed to give a compound of Formula XII.

The hydrolysis of compound of Formula VIII to give a compound of Formula XII can be carried out in lithium hydroxide, potassium hydroxide or sodium hydroxide in one or more solvents, for example, tetrahydrofuran, water, methanol, dichloromethane, acetone, acetonitrile or dioxane optionally in the presence of an acid, for example, trifluoroacetic acid.

A compound of Formula XV can be prepared by following Scheme III as follows.

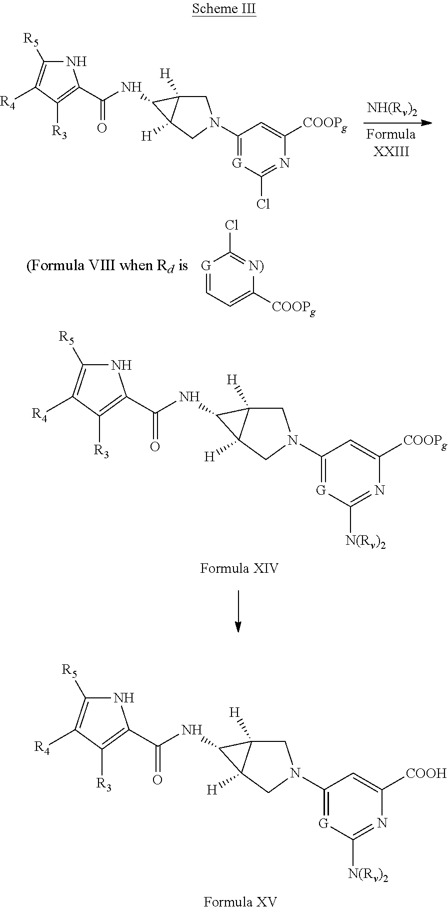

The compound of Formula VIII (when R_{d} is

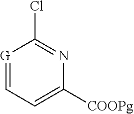

and G is N or CH) can be reacted with a compound of Formula XXIII (wherein both are independently hydrogen or alkyl or two Rᵥ along with the N to which they are attached can join together to form a heterocyclic ring optionally containing heteroatoms O, N or S) to give a compound of Formula XIV, which can then deprotected to give a compound of Formula XV.

The reaction of compound of Formula VIII with a compound of Formula XIII to give a compound of Formula XIV can be carried out in the presence of a base, for example, triethylamine, sodium hydride, pyridine, sodium hydrogen carbonate, potassium carbonate, cesium carbonate, sodium acetate, sodium thiosulfate or diisopropyl ethylamine in one or more solvent, for example, dimethylformamide, dimethylsulfoxide, dioxane or tetrahydrofuran.

The hydrolysis of compound of Formula XIV to give a compound of Formula XV can be carried out in lithium hydroxide, potassium hydroxide or sodium hydroxide in one or more solvents, for example, tetrahydrofuran, water, acetone, acetonitrile or dioxane.

The compound of Formula XVI can be prepared by following the Scheme IV as follows

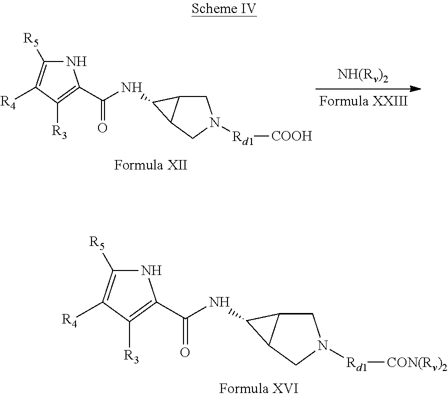

Accordingly, the compound of Formula XII (wherein R_{d1} is as defined earlier) can undergo coupling with a compound of Formula XXIII (wherein Rᵥ is as defined earlier) to give a compound of Formula XVI.

Coupling of compound of Formula XII with a compound of Formula XXIII to give a compound of Formula XVI can be carried out in one or more solvent, for example, dimethylformamide, tetrahydrofuran or dioxane using a coupling agent, for example, 1-ethyl-3-(3′-dimethylaminopropyl)carbodiimide hydrochloride (EDCI), 1,3-dicyclohexyl-carbodiimide (DCC), N-[(dimethylamino)-1H-1,2,3-triazolo[4,5-b]pyridylmethylene]-N-methylmethanaminium hexafluorophosphate N-oxide (HATU) or benzotriazol-1-yl-N-oxy-tris(pyrrolidino)phosphonium hexafluorophosphate (PyBOP) and, optionally, a catalyst, for example, 1-hydroxybenzotriazole (HOBt), N-hydroxy succinimide (HOSu), N-hydroxy-5-norbene-endo-2,3-dicarboxamide (HONB) 3-hydroxy-3,4-dihydro-4-oxo-1,2,3-benzotriazine (HODhbt) or 7-aza-1-hydroxybenzotriazole (HOAt) and, optionally, with a base, for example, N-methyl-morpholine (NMM), N,N-dimethylaminopyridine (DMAP), triethylamine (TEA) or N,N-diisopropylethylamine (DIEA). The compound of Formula XXI can be prepared by following the Scheme V as follows:

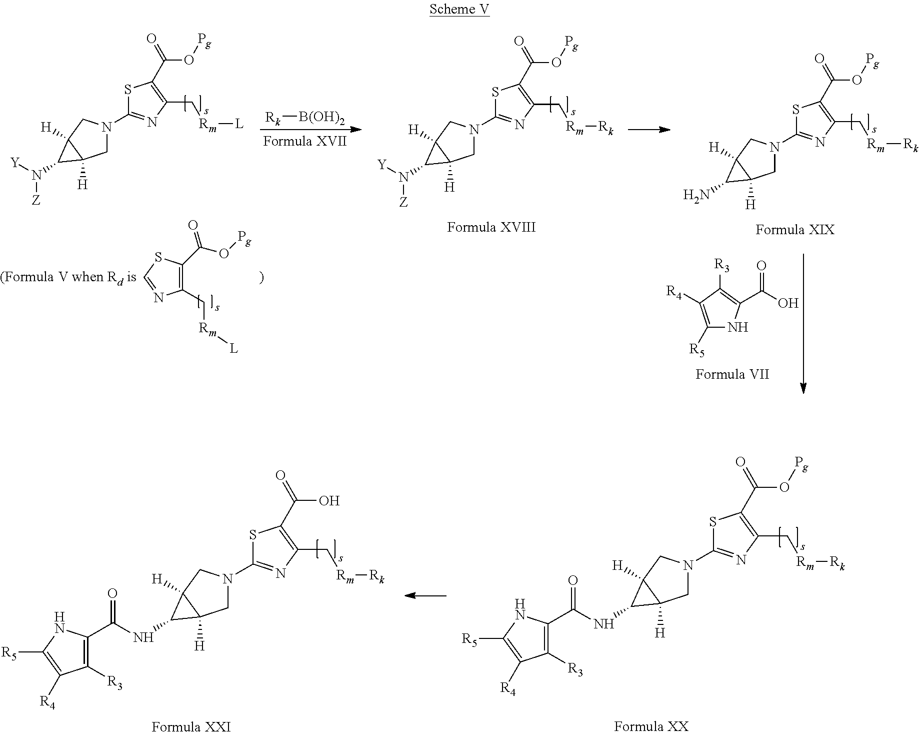

Accordingly, the compound of Formula V (when R_{d} is

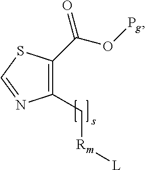

Rₘ is aryl or heteroaryl, s can be 0 or 1, and L is as defined earlier) can undergo Suzuki coupling with a compound of Formula XVII (wherein R_{λ} can be (un)substituted aryl or heteroaryl and y, Z and P_{g} are as defined earlier) to give a compound of Formula XVIII. The compound of Formula XVIII can undergo N-deprotection to give a compound of Formula XIX. The compound of Formula XIX can undergo coupling to give a compound of Formula XX. The compound of Formula XX can undergo hydrolysis to give a compound of Formula XXI.

The coupling of compound of Formula V with a compound of Formula XVII to give a compound of Formula XVIII can be carried out in the presence of bis-(diphenyl-phosphino)ferrocene palladium II dichloride (Pd(dppf)Cl₂, tetrakistriphenylphosphine palladium (0) [Pd (Ph₃P)₄], palladium acetate or dichlorobistriphenylphosphine palladium (II), with a suitable base, for example, potassium carbonate, sodium acetate or potassium acetate in one or more solvent, for example, acetonitrile, dimethylformamide, toluene, tetrahydrofuran, acetone or dioxane.

N-deprotection of compound of Formula XVIII to give a compound of Formula XIX can be carried out in the similar way as the deprotection of compound of Formula V to give a compound of Formula VI.

The coupling of compound of Formula XIX with a compound of Formula VII to give a compound of Formula XX can be carried out in a similar way as the coupling of compound of Formula VI with a compound of Formula VII to give a compound of Formula VIII.

The hydrolysis of compound of Formula XX to give a compound of Formula XXI can be carried out in the similar way as the hydrolysis of compound of Formula VIII to give a compound of Formula XII.

The compound of Formula XXV can be prepared by following the Scheme VI as follows

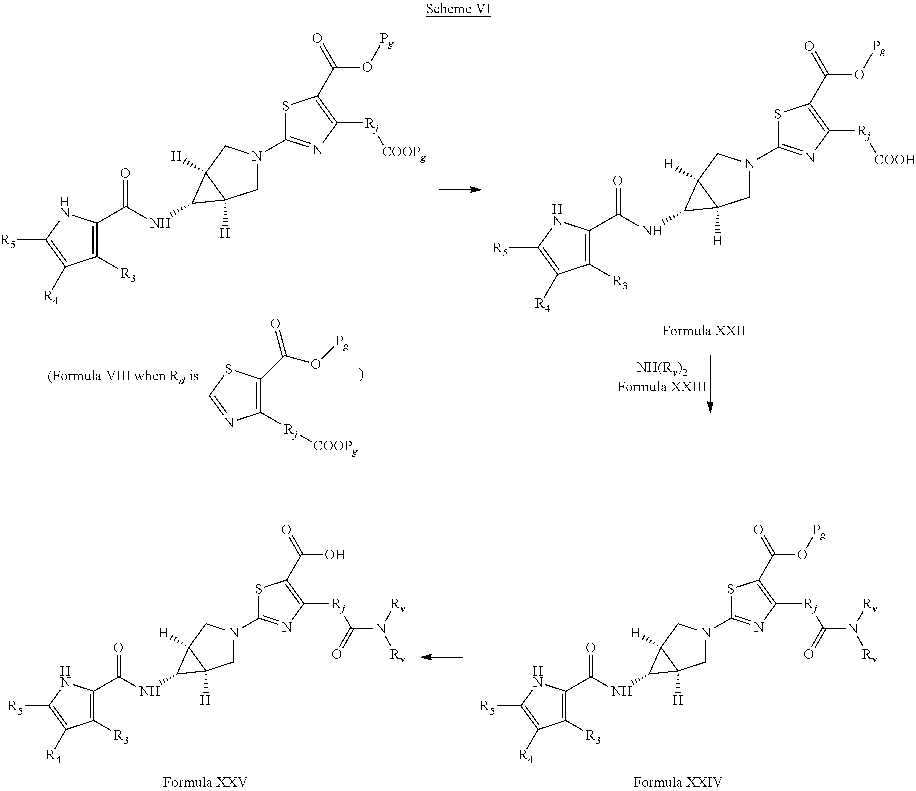

Accordingly, the compound of Formula VIII (when R_{d} is

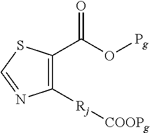

wherein Rⱼ is methylene or benzylene and P_{g} is as defined earlier) undergo hydrolysis to give a compound of Formula XXII. The compound of Formula XXII undergo coupling with a compound of Formula XXIII (wherein both Rᵥ are independently hydrogen or alkyl or two Rvs along with the N to which they are attached can join together to form a heterocyclic ring optionally containing heteroatoms O, N or S) to give a compound of Formula XXIV. The compound of Formula XXIV undergo hydrolysis to give a compound of Formula XXV.

Hydrolysis of compound of Formula VIII to give a compound of Formula XXII can be carried out with sodium hydroxide, lithium hydroxide or potassium hydroxide in one or more solvents, for example, methanol, tetrahydrofuran, water, acetone, acetonitrile or dioxane.

The coupling of compound of Formula XXII with a compound of Formula XXIII to give a compound of Formula XXIV can be carried out in the same way as the coupling of compound of Formula XII to give a compound of Formula XVI.

Hydrolysis of compound of Formula XXIV to give a compound of Formula XXV can be carried out in the similar way as the hydrolysis of compound of Formula VIII to give a compound of Formula XII.

The compounds of Formula XXVIII, XXX and XXXII can be prepared by following the synthetic route as given below

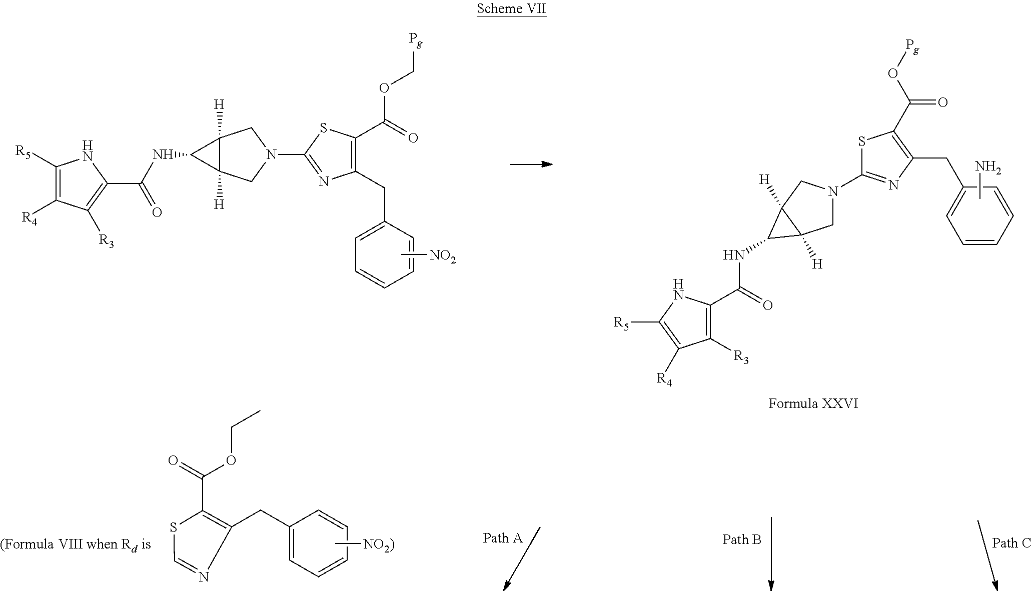

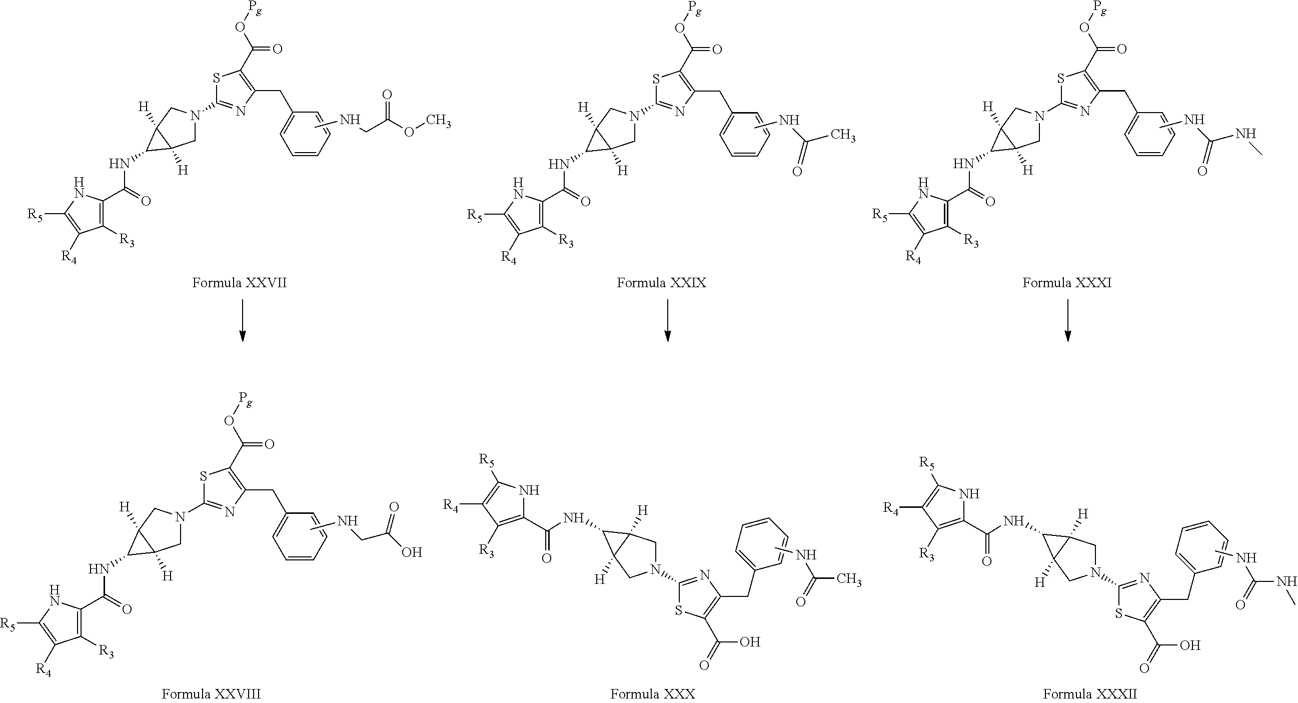

Accordingly, the compound of Formula VIII (when R_{d} is

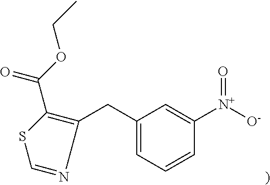

undergo reduction to give a compound of Formula XXVI. The compound of Formula XXVI can be reacted through 3 pathways.
Path A: The compound of Formula XXVI can be reacted with methyl bromoacetate to give a compound of Formula XXVII. The compound of Formula XXVII undergo hydrolysis to give a compound of Formula XXVIII
Path B: The compound of Formula XXVI can be reacted with acetyl chloride to give a compound of Formula XXIX. The compound of Formula XXIX undergo hydrolysis to give a compound of Formula XXX.
Path C: The compound of Formula XXVI can be reacted with p-nitrophenyl chloroformate to give a compound of Formula XXXI. The compound of Formula XXXI undergo hydrolysis to give a compound of Formula XXXII.

Reduction of compound of Formula VIII to give a compound of Formula XXVI can be carried out by one or more reducting agent. For example, stannous chloride, titanium(III) chloride, sodium hydrosulfite, Fe/HCl, sodium sulfide or by catalytic hydrogenation using palladium-on-carbon, platinum(IV) oxide, or Raney nickel optionally in the presence of an acid, for example, hydrochloric acid or hydrobromic acid.

### Path A:

The reaction of compound of Formula XXVI with methyl bromoacetate to give a compound of Formula XXVII can be carried out in the presence of one or more base, for example, N,N-diisopropylethylamine (DIEA), N-methyl-morpholine (NMM), N,N-dimethylaminopyridine (DMAP) or triethylamine (TEA) in the presence of one or more solvent, for example, dimethylformamide, tetrahydrofuran, dimethylsulfoxide, dioxane or acetonitrile.

Hydrolysis of compound of Formula XXVII to give a compound of Formula XXVIII can be carried out in the similar way as the hydrolysis of compound of Formula VIII to a compound of Formula XXII.

### Path B:

The reaction of compound of Formula XXVI with acetyl chloride to give a compound of Formula XXIX can be carried out in one or more solvent, for example, tetrahydrofuran, methanol, acetone, acetonitrile or dioxane in the presence of an amine, for example, triethyl amine, N-methyl-morpholine (NMM), N,N-dimethylaminopyridine (DMAP) or N,N-diisopropylethylamine (DIEA).

Hydrolysis of compound of Formula XXIX to give a compound of Formula XXX can be carried out in the similar way as the hydrolysis of compound of Formula VIII to a compound of Formula XXII.

### Path C:

The reaction of compound of Formula XXVI with p-nitrophenyl chloroformate to give a compound of Formula XXXI can be carried out in the presence of a base, for example, pyridine, sodium hydride, potassium carbonate, sodium acetate, sodium thiosulfate, sodium hydrogen carbonate, or diisopropyl ethylamine in one or more solvent, for example, tetrahydrofuran, dimethylsulfoxide, dioxane, dimethylformamide or acetonitrile.

Hydrolysis of compound of Formula XXXI to give a compound of Formula XXXII can be carried out in the similar way as the hydrolysis of compound of Formula VIII to a compound of Formula XXII.

In the above schemes, where specific reagents, for example, bases, acids, solvents, condensing agents, acylating agents, hydrolyzing agents, metal catalysts etc., are mentioned, are it is to be understood that other reagents, e.g., other acids, bases, solvents, condensing agents, reducing agent, deprotecting agent, hydrolyzing agents, metal catalysts etc., known to one of ordinary skill in the art may be used. Similarly, reaction temperatures and durations may be adjusted according to the desired needs without undue experimentation and well within the abilities of one of ordinary skill in the art. All the epimers, unless otherwise specified in the above schemes, are also encompassed within the scope of the invention.

Table-1 lists the type of compounds synthesized by using the synthetic procedure as demonstrated in Schemes 1-VII.

TABLE I
Formula 1

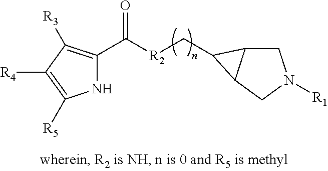

| S. | | | |
|---|---|---|---|
| No. | R₁ | R₃ | R₄ |
| 1 | 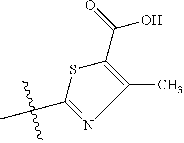 | Cl | Cl |
| 2 | 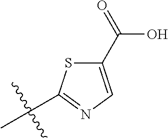 | Cl | Cl |
| 3 | 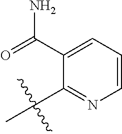 | H | Cl |
| 4 | 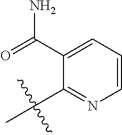 | Cl | Cl |
| 5 | 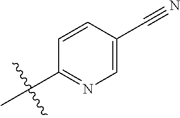 | Cl | Cl |
| 6 | 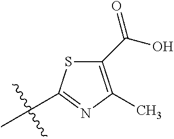 | H | Cl |
| 7 | 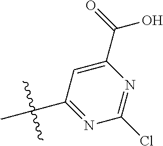 | Cl | Cl |
| 8 | 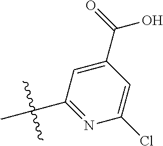 | Cl | Cl |
| 9 | 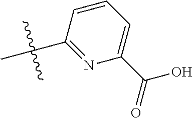 | Cl | Cl |
| 10 | 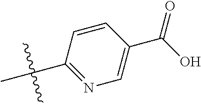 | Cl | Cl |
| 11 | 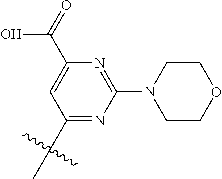 | Cl | Cl |
| 12 | 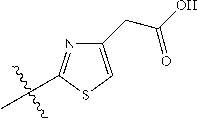 | Cl | Cl |
| 13 | 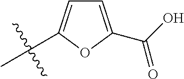 | Cl | Cl |
| 14 | 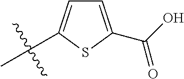 | Cl | Cl |
| 15 | 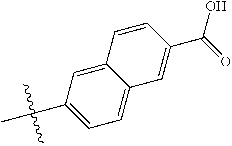 | Cl | Cl |
| 16 | 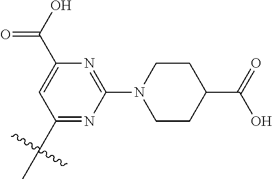 | Cl | Cl |
| 17 | 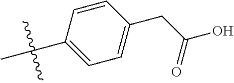 | Cl | Cl |
| 18 | 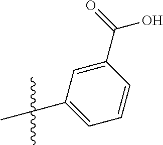 | Cl | Cl |
| 19 | 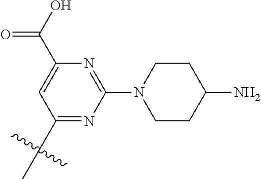 | Cl | Cl |
| 20 | 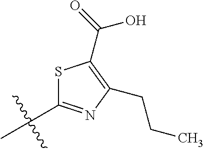 | Cl | Cl |
| 21 | 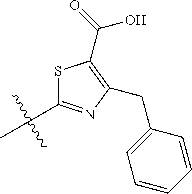 | Cl | Cl |
| 22 | 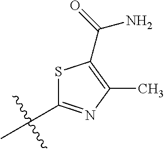 | Cl | Cl |
| 23 | 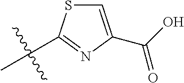 | Cl | Cl |
| 24 | 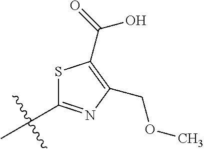 | Cl | Cl |
| 25 | 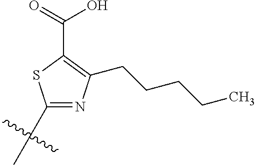 | Cl | Cl |
| 26 | 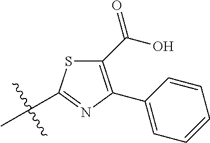 | Cl | Cl |
| 27 | 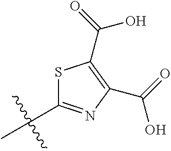 | Cl | Cl |
| 28 | 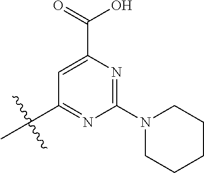 | Cl | Cl |
| 29 | 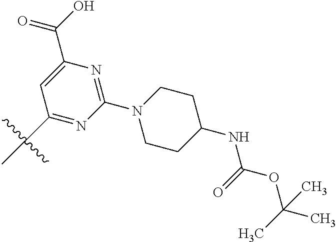 | Cl | Cl |
| 30 | 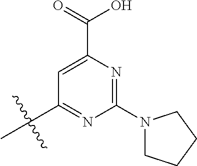 | Cl | Cl |
| 31 | 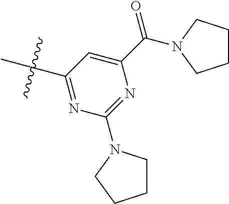 | Cl | Cl |
| 32 | 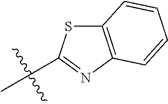 | Cl | Cl |
| 33 | 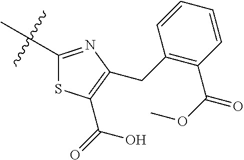 | Cl | Cl |
| 34 | 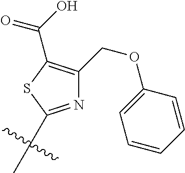 | Cl | Cl |
| 35 | 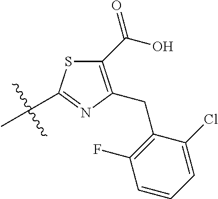 | Cl | Cl |
| 36 | 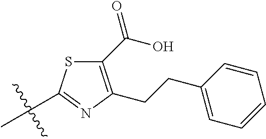 | Cl | Cl |
| 37 | 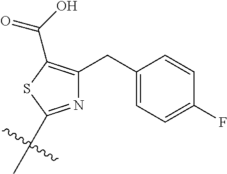 | Cl | Cl |
| 38 | 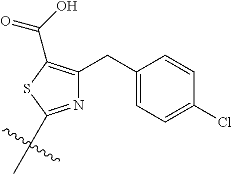 | Cl | Cl |
| 39 | 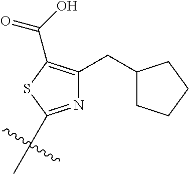 | Cl | Cl |
| 40 | 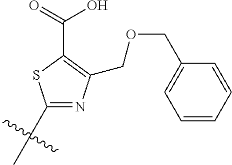 | Cl | Cl |
| 41 | 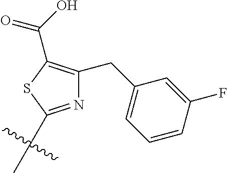 | Cl | Cl |
| 42 | 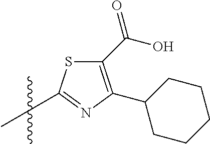 | Cl | Cl |
| 43 | 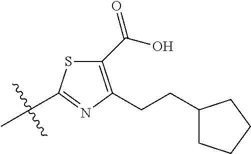 | Cl | Cl |
| 44 | 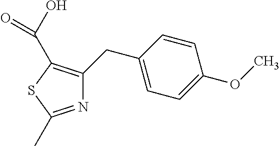 | Cl | Cl |
| 45 | 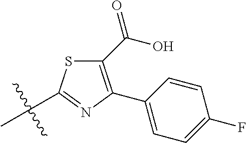 | Cl | Cl |
| 46 | 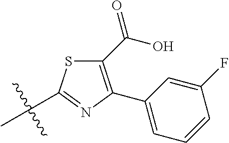 | Cl | Cl |
| 47 | 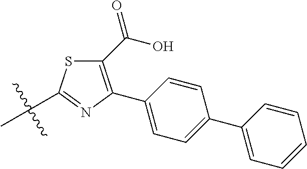 | Cl | Cl |
| 48 | 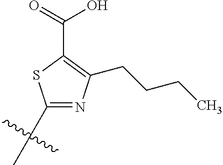 | Cl | Cl |
| 49 | 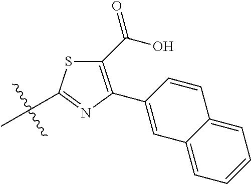 | Cl | Cl |
| 50 | 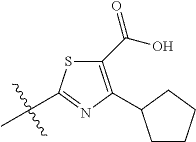 | Cl | Cl |
| 51 | 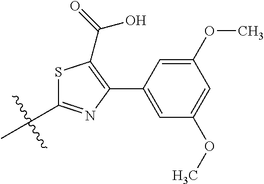 | Cl | Cl |
| 52 | 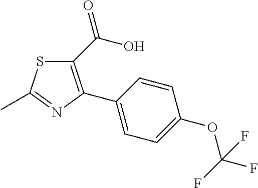 | Cl | Cl |
| 53 | 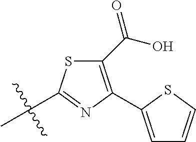 | Cl | Cl |
| 54 | 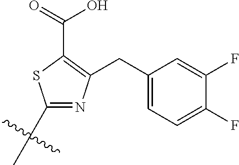 | Cl | Cl |
| 55 | 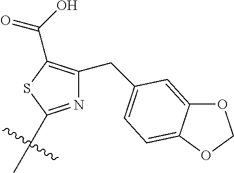 | Cl | Cl |
| 56 | 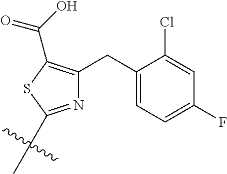 | Cl | Cl |
| 57 | 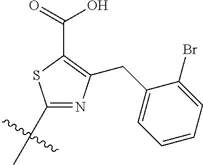 | Cl | Cl |
| 58 | 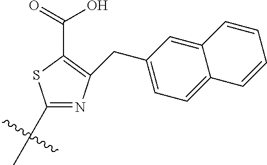 | Cl | Cl |
| 59 | 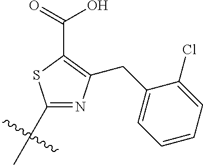 | Cl | Cl |
| 60 | 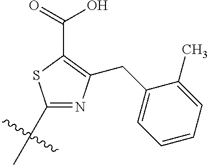 | Cl | Cl |
| 61 | 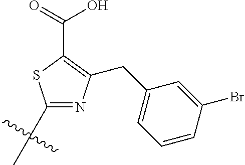 | Cl | Cl |
| 62 | 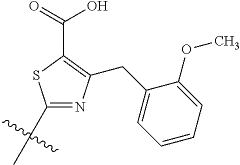 | Cl | Cl |
| 63 | 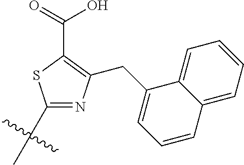 | Cl | Cl |
| 64 | 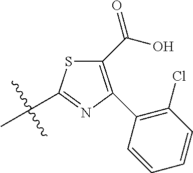 | Cl | Cl |
| 65 | 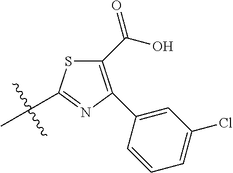 | Cl | Cl |
| 66 | 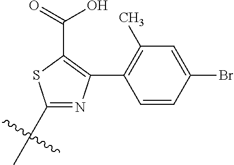 | Cl | Cl |
| 67 | 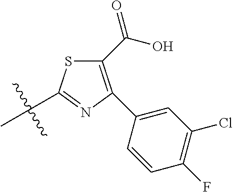 | Cl | Cl |
| 68 | 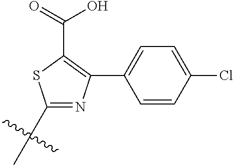 | Cl | Cl |
| 69 | 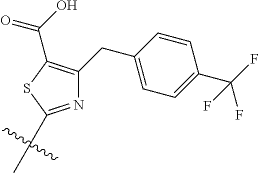 | Cl | Cl |
| 70 | 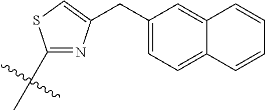 | Cl | Cl |
| 71 | 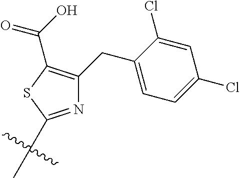 | Cl | Cl |
| 72 | 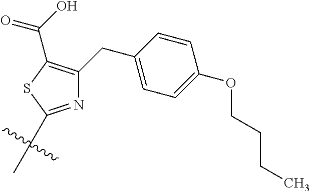 | Cl | Cl |
| 73 | 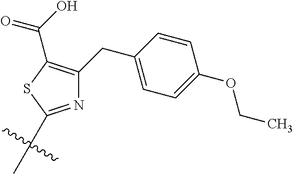 | Cl | Cl |
| 74 | 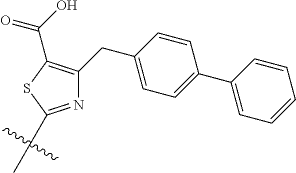 | Cl | Cl |
| 75 | 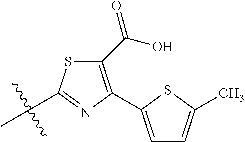 | Cl | Cl |
| 76 | 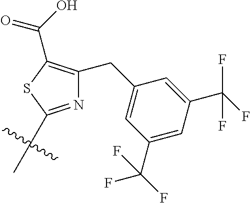 | Cl | Cl |
| 77 | 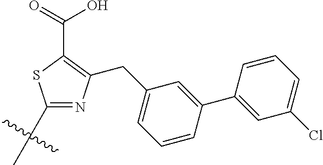 | Cl | Cl |
| 78 | 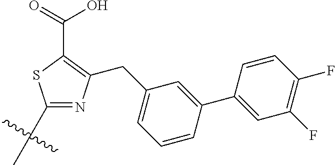 | Cl | Cl |
| 79 | 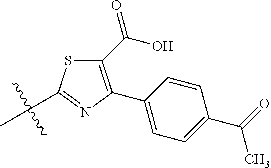 | Cl | Cl |
| 80 | 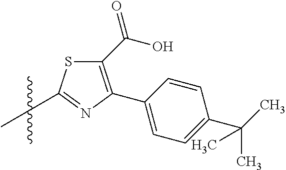 | Cl | Cl |
| 81 | 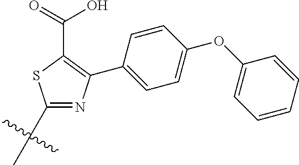 | Cl | Cl |
| 82 | 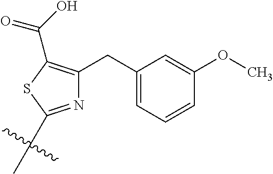 | Cl | Cl |
| 83 | 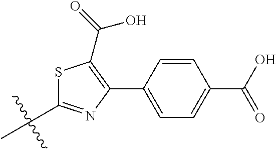 | Cl | Cl |
| 84 | 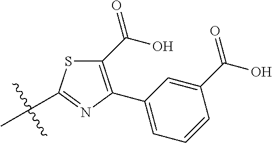 | Cl | Cl |
| 85 | 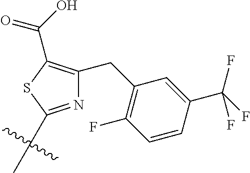 | Cl | Cl |
| 86 | 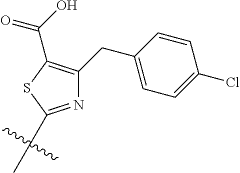 | Cl | Cl |
| 87 | 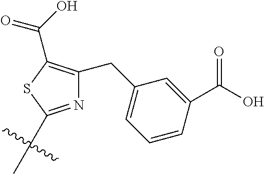 | Cl | Cl |
| 88 | 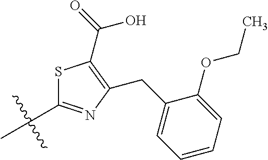 | Cl | Cl |
| 89 | 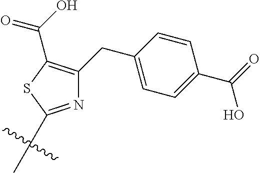 | Cl | Cl |
| 90 | 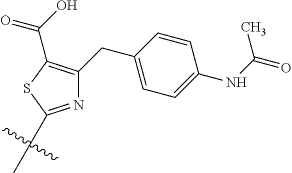 | Cl | Cl |
| 91 | 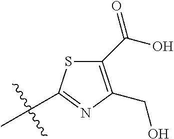 | Cl | Cl |
| 92 | 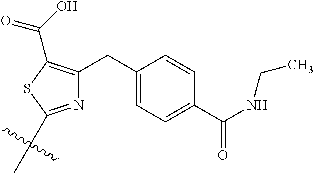 | Cl | Cl |
| 93 | 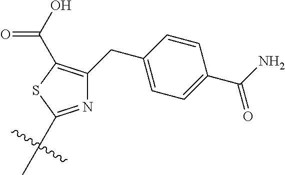 | Cl | Cl |
| 94 | 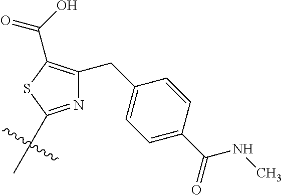 | Cl | Cl |
| 95 | 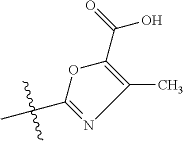 | Cl | Cl |
| 96 | 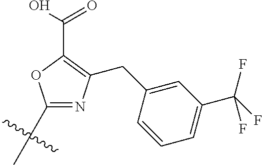 | Cl | Cl |
| 97 | 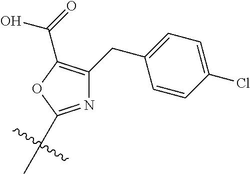 | Cl | Cl |
| 98 | 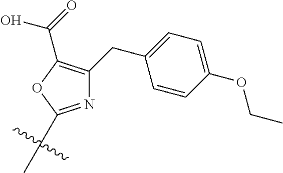 | Cl | Cl |
| 99 | 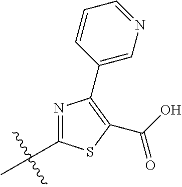 | Cl | Cl |
| 100 | 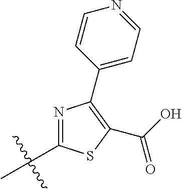 | Cl | Cl |
| 101 | 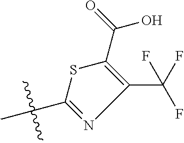 | Cl | Cl |
| 102 | 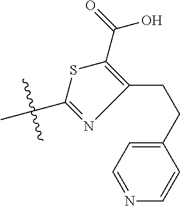 | Cl | Cl |
| 103 | 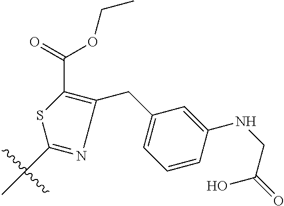 | Cl | Cl |
| 104 | 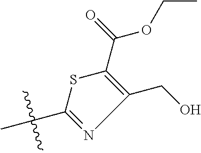 | Cl | Cl |
| 105 | 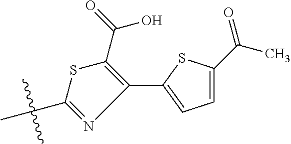 | Cl | Cl |
| 106 | 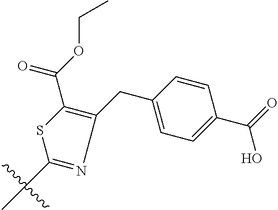 | Cl | Cl |
| 107 | 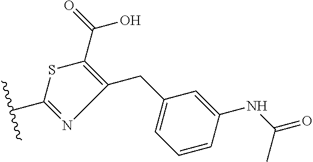 | Cl | Cl |
| 108 | 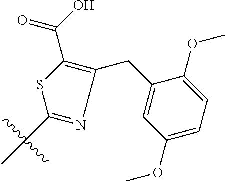 | Cl | Cl |
| 109 | 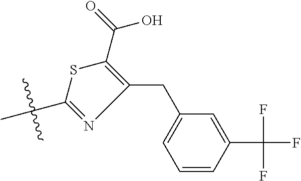 | Cl | Cl |
| 110 | 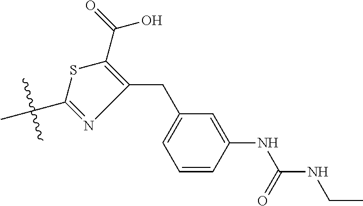 | Cl | Cl |
| 111 | 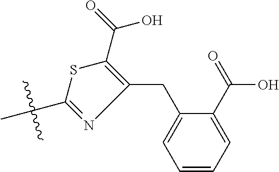 | Cl | Cl |
| 112 | 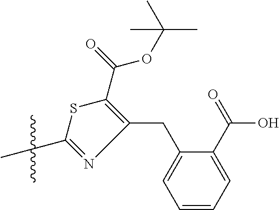 | Cl | Cl |
| 113 | 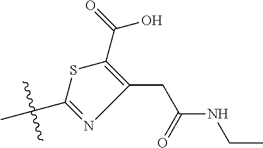 | Cl | Cl |
| 114 | 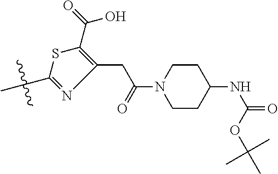 | Cl | Cl |
| 115 | 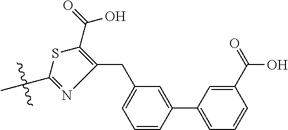 | Cl | Cl |
| 116 | 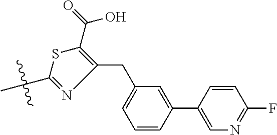 | Cl | Cl |
| 117 | 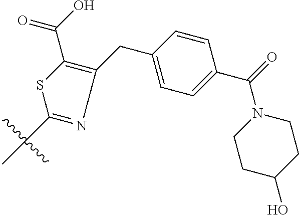 | Cl | Cl |
| 118 | 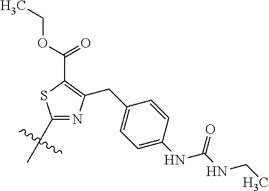 | Cl | Cl |
| 119 | 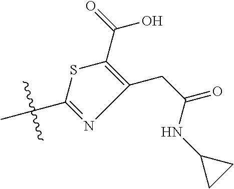 | Cl | Cl |
| 120 | 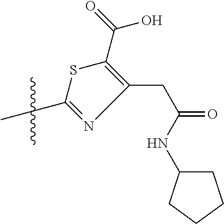 | Cl | Cl |
| 121 | 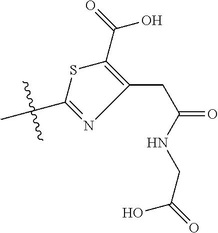 | Cl | Cl |
| 122 | 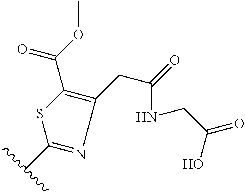 | Cl | Cl |
| 123 | 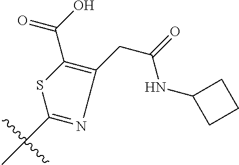 | Cl | Cl |
| 124 | 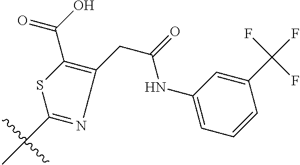 | Cl | Cl |
| 125 | 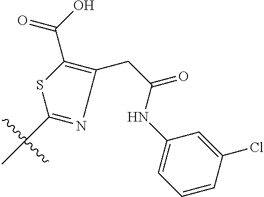 | Cl | Cl |
| 126 | 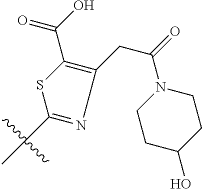 | Cl | Cl |
| 127 | 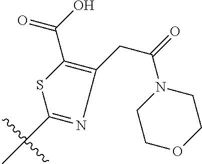 | Cl | Cl |
| 128 | 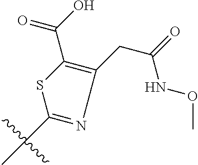 | Cl | Cl |
| 129 | 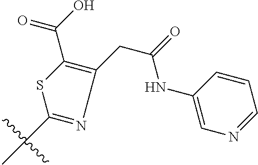 | Cl | Cl |
| 130 | 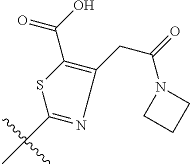 | Cl | Cl |
| 131 | 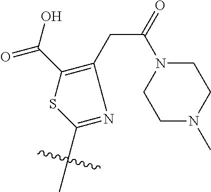 | Cl | Cl |
| 132 | 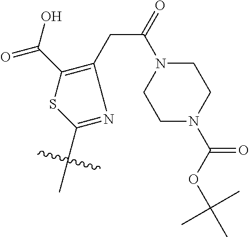 | Cl | Cl |
| 133 | 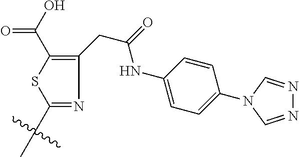 | Cl | Cl |
| 134 | 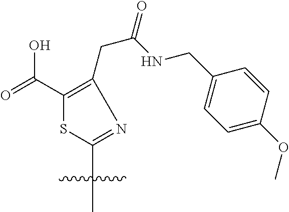 | Cl | Cl |
| 135 | 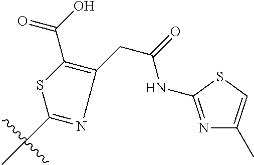 | Cl | Cl |
| 136 | 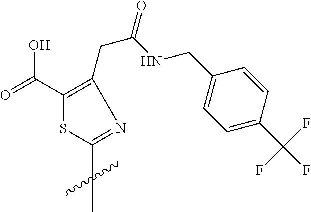 | Cl | Cl |
| 137 | 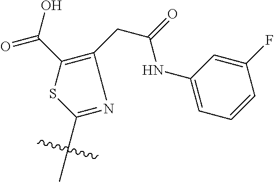 | Cl | Cl |
| 138 | 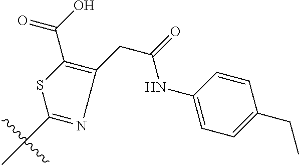 | Cl | Cl |
| 139 | 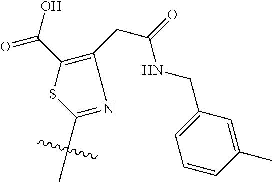 | Cl | Cl |
| 140 | 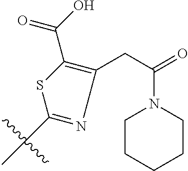 | Cl | Cl |
| 141 | 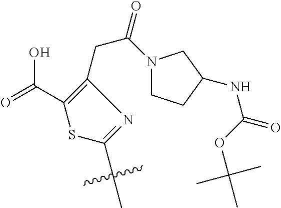 | Cl | Cl |
| 142 | 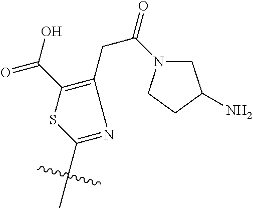 | Cl | Cl |
| 143 | 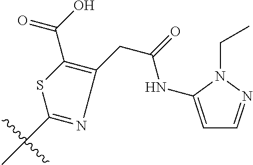 | Cl | Cl |
| 144 | 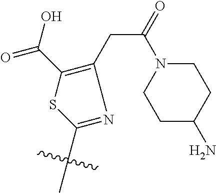 | Cl | Cl |
| 145 | 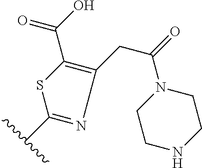 | Cl | Cl |
| 146 | 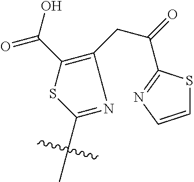 | Cl | Cl |
| 147 | 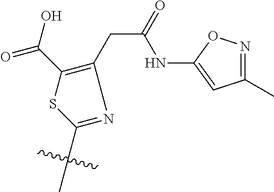 | Cl | Cl |
| 148 | 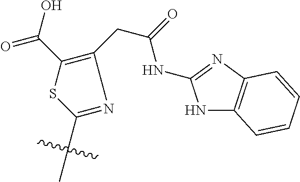 | Cl | Cl |
| 149 | 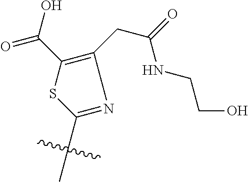 | Cl | Cl |
| 150 | 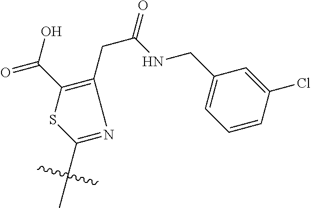 | Cl | Cl |
| 151 | 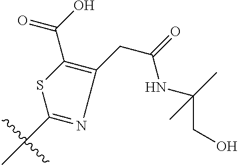 | Cl | Cl |
| 152 | 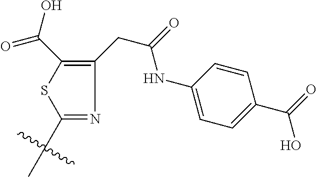 | Cl | Cl |
| 153 | 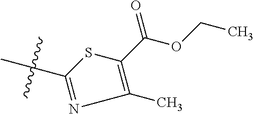 | Cl | Cl |
| 154 | 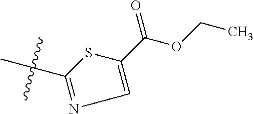 | Cl | Cl |
| 155 | 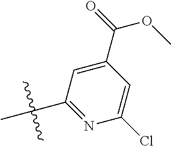 | Cl | Cl |
| 156 | 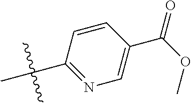 | Cl | Cl |
| 157 | 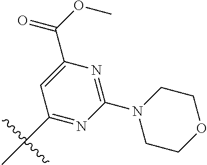 | Cl | Cl |
| 158 | 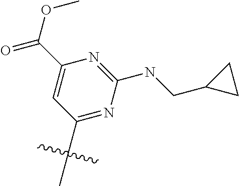 | Cl | Cl |
| 159 | 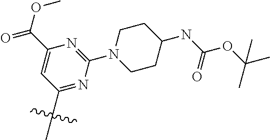 | Cl | Cl |
| 160 | 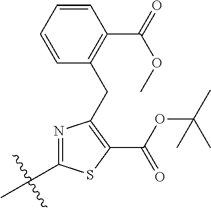 | Cl | Cl |
| 161 | 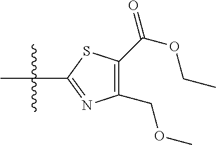 | Cl | Cl |
| 162 | 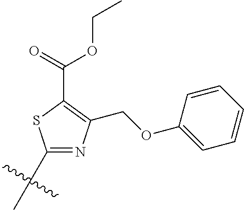 | Cl | Cl |
| 163 | 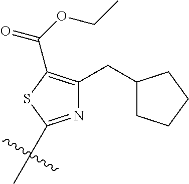 | Cl | Cl |
| 164 | 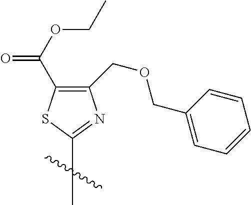 | Cl | Cl |
| 165 | 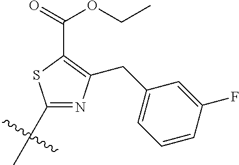 | Cl | Cl |
| 166 | 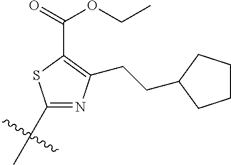 | Cl | Cl |
| 167 | 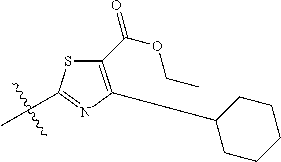 | Cl | Cl |
| 168 | 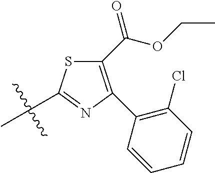 | Cl | Cl |
| 169 | 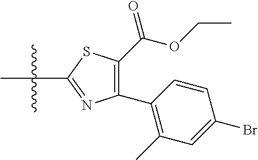 | Cl | Cl |
| 170 | 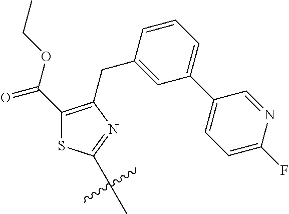 | Cl | Cl |
| 171 | 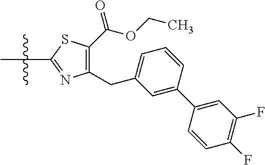 | Cl | Cl |
| 172 | 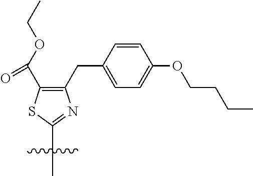 | Cl | Cl |
| 173 | 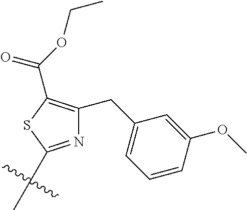 | Cl | Cl |
| 174 | 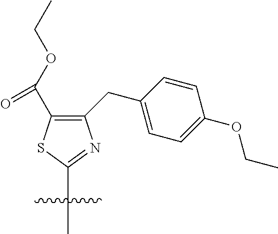 | Cl | Cl |
| 175 | 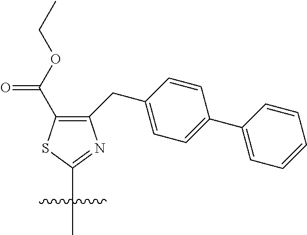 | Cl | Cl |
| 176 | 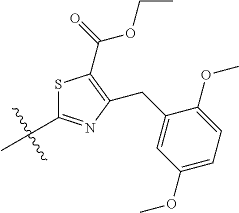 | Cl | Cl |
| 177 | 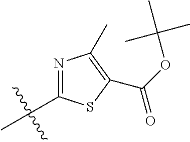 | Cl | Cl |
| 178 | 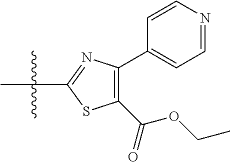 | Cl | Cl |
| 179 | 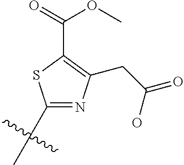 | Cl | Cl |

The compounds described herein may be administered to an animal for treatment orally, topically, rectally, internasally, or by parenteral route. Pharmaceutical compositions disclosed herein comprise pharmaceutically effective amounts of compounds described herein formulated together with one or more pharmaceutically acceptable carriers, excipients or diluents.

Solid form preparations for oral administration include capsules, tablet, pills, powder, granules, lozenges, troches, cachets and suppositories. For solid form preparations, active compounds can be mixed with one or more inert, pharmaceutically acceptable excipients or carrier, for example, sodium citrate, dicalcium phosphate and/or fillers or extenders (for example, starches, lactose, sucrose, glucose, mannitol, silicic acid or mixtures thereof); binders, for example, carboxymethylcellulose, alginates, gelatins, polyvinylpyrrolidinone, sucrose, acacia or mixtures thereof; disintegrating agents, for example, agar-agar, calcium carbonate, potato starch, alginic acid, certain silicates, sodium carbonate or mixtures thereof; absorption acceletors, for example, quaternary ammonium compounds; wetting agents, for example, cetyl alcohol, glycerol mono stearate or mixtures thereof; adsorbants, for example, Kaolin; lubricants, for example, talc, calcium stearate, magnesium stearate, solid polyethyleneglycol, sodium lauaryl sulfate or mixtures thereof.

Capsules, tablets or pills may also comprise buffering agents.

Tablets, capsules, pills or granules can be prepared using one or more coatings or shells to modulate the release of active ingredients, for example, enteric coatings or other coatings known to one of ordinary skill in the art.

Liquid form preparations for oral administration include pharmaceutically acceptable emulsions, solutions, suspensions, syrups or elixirs. In such liquid form preparations, active compounds can be mixed with water or one or more non-toxic solvents, solubilizing agents or emulsifiers, for example, water, ethyl alcohol, isopropyl alcohol, ethyl carbonate, ethyl acetate, benzyl alcohol, benzyl benzoate, propylene glycol, 1,3-butylene glycol, dimethylformamide, oils, for example, cottonseed, groundnut, corn, germ, olive, castor and sesame oil, glycerol, fatty acid esters of sorbitan or mixtures thereof. Oral compositions can also include one or more adjuvants, for example, wetting agents, emulsifying agents, suspending agents, sweetening agents, flavoring agents, perfuming agents or mixtures thereof.

Injectable preparations, for example, sterile injections, and aqueous suspensions may be formulated according to methods known to one of ordinary skill in the art, and in particular, using one or more suitable dispersing or wetting and suspending agents. Acceptable vehicles and solvents that may be employed include one or more of water, Ringer's solution, isotonic sodium chloride or mixtures thereof.

Suppositories for rectal administration of the compound of this invention can be prepared by mixing the drug with suitable nonirritating excipients such as coca butter and polyethylene glycols, which are solid at ordinary temperatures but liquid at body temperature an which therefore melt in the rectum and release the drug

Dosage forms for topical or transdermal administration of a compound of the present invention include ointments, pastes, creams, lotions, gels, powders, solutions, sprays, inhalants or patches. Active compounds can be admixed under sterile condition with one or more pharmaceutically acceptable carriers and optionally any preservatives or buffers as may be required. Ophthalmic formulations, eardrops, eye ointments, powders and solutions are also encompassed within the scope of this invention.

Pharmaceutical preparations may be in unit dosage form. In unit dosage form, the preparations can be subdivided into unit doses containing appropriate quantities of active components. Unit dosage forms can be packaged preparations containing discrete capsules, powders, in vials or ampoules, ointments, capsules, sachets, tablets, gels, creams or any combination and number of such packaged forms.

The following examples are set forth to demonstrate general synthetic procedures for the preparation of representative compounds. The examples are provided to illustrate particular aspect of the disclosure and do not limit the scope of the present invention.

### EXPERIMENTAL PROCEDURE

Various solvents, for example, dimethylformamide, benzene, tetrahydrofuran, acetonitrile, dichloromethane etc were dried using various drying reagents according to procedure as described in the literature.

### Synthesis of Intermediates

### Synthesis of 4-Chloro-5-methyl-1H-pyrrole-2-carboxylic acid

### Step I: Synthesis of Ethyl-5-methyl-1H-pyrrole-2-carboxylate

The title compound was prepared as per the procedure given in JOC, 1996, 61, 9068 EIMS m/z 154.06 [M+H]⁺

### Step II: Synthesis of ethyl-4-chloro-5-methyl-1H-pyrrole-2-carboxylate

The title compound was prepared as per the procedure given in WO2005026149

EIMS m/z 188.09 [M+H]⁺

### Step III: Synthesis of 4-Chloro-5-methyl-1H-pyrrole-2-carboxylic acid

A mixture of ethyl-4-chloro-5-methyl-pyrrole-2-carboxylate (3 g, 16 mmol) in a mixture of tetrahydrofuran:water (3:1, 40 ml) was treated with lithium hydroxide (6.7 g, 160 mmol). The reaction mixture was heated to about 80° C. and stirred for about 16 hours. The solvent was removed under vacuum, the resultant mixture was cooled to about 0° C. and then acidified with 30% hydrochloric acid, extracted with ethyl acetate. The combined organics were washed with water, brine, dried over anhydrous sodium sulphate and concentrated to afford 4-Chloro-5-methyl-1H-pyrrole-2-carboxylic acid (1.8 g).

EIMS m/z 160.04 [M+H]⁺

### Synthesis of 3,4-dichloro-5-methyl-1H-pyrrole-2-carboxylic acid

### Step I: Synthesis of Ethyl-2,4-Dichloro-5-methyl-1H-pyrrole-2-carboxylate

The title compound was prepared from Ethyl-5-methyl-1H-pyrrole-2-carboxylate as per the procedure given in WO2005026149

EIMS m/z 222.42 [M+11]⁺

### Step II: Synthesis of 2,4-Di-Chloro-5-methyl-1H-pyrrole-2-carboxylic acid

The title compound was prepared as per the procedure followed for synthesis of 4-Chloro-5-methyl-1H-pyrrole-2-carboxylic acid.

EIMS m/z 194.09 [M+H]⁺

### Synthesis of tert-butyl[(1R,5S,6s)-3-benzyl-3-azabicyclo[3.1.0]hex-6-yl]carbamate

The title compound was prepared as per the procedure known in the art (Braish, Tamim F.; Castaldi, Michael; Chan, Samantha; Fox, Darell E.; Keltonic, Tom; McGarry, James; Hawkins, Joel M.; Norris, Timothy; Rose, Peter R.; et al. Construction of the (1α,5α,6α)-6-amino-3-azabicyclo[3.1.0]hexane ring system. Synlett (1996), (11), 1100-1102 example 7 on page 1101.

### Synthesis of di-tert-butyl[(1R,5S,6s)-3-benzyl-3-azabicyclo[3.1.0]hex-6-yl]imidodicarbonate

To a solution of tert-butyl[(1R,5S,6s)-3-benzyl-3-azabicyclo[3.1.0]hex-6-yl]carbamate (10 g, 34.7 mmol) in dry toluene (150 ml), di-tert-butyl dicarbonate (10.5 g, 48.6 mmol), triethylamine (9.43 ml, 68 mmol), and 4-dimethylaminopyridine (1.2 g, 10.4 mmol) were added and this reaction mixture was refluxed at about 110° C. under an inert atmosphere for about 16 hours. The reaction mixture was then concentrated under vacuum, diluted with ethyl acetate, washed with water, brine and dried over anhydrous sodium sulfate and concentrated under vacuum. The residue was purified by column chromatography over SiO₂ using hexane/ethyl acetate system as eluent to afford the title compound (12 g).

EIMS m/z 388.21 [M+H]⁺

### Synthesis of tert-butyl[(1R,5S,6s)-3-(trifluoroacetyl)-3-azabicyclo[3.1.0]hex-6-yl]carbamate

A solution of tert-butyl (1R,5S,6s)-3-azabicyclo[3.1.0]hex-6-ylcarbamate (10 g, 50 mmol)) and triethylamine (21 ml, 151 mmol) in dichloromethane (100 ml) was cooled at about 0° C. and treated drop wise with trifluoroacetic anhydride (15.9 g, 75 mmol). The reaction mixture was stirred at room temperature (˜25° C.) for about 16 hours, washed with water, brine, dried over anhydrous sodium sulfate and concentrated to obtain a solid residue that was triturated with hexane to afford the desired compound (12 g).

EIMS m/z 294 [M+H]⁺

### Synthesis of 2-bromo thiazoles

### Synthesis of ethyl 2-bromo-4-(4-cyanophenyl)-1,3-thiazole-5-carboxylate

### Step I: Synthesis of 4-[(2,2-dimethyl-4,6-dioxo-1,3-dioxan-5-ylidene)(hydroxy)methyl]benzonitrile

A solution of meldrum's acid (4.06 g, 28.2 mmol) in dichloromethane (100 ml) was treated with 1-ethyl-3-(3-dimethylaminopropyl)carbodiimide hydrochloride (5.4 g, 28.2 mmol), and 4-dimethylaminopyridine (7.16 g, 58.75 mmol) and 4-cyanobenzoic acid (3.45 g, 23.5 mmol). The mixture was stirred at room temperature (˜25° C.) for about 24 hours. The reaction mixture was concentrated to remove dichloromethane, residue taken up in ethyl acetate (150 ml) and washed with 5% aqueous KHSO4 solution (4×70 ml), followed by washing with 5% aqueous Sodium bicarbonate solution (1×50 ml), water (1×50 ml), brine (1×50 ml), dried and concentrated to afford the product (3.7 g) that was taken as such to the next step.

EIMS m/z 274.52 [M+H]⁺

### Step II: Synthesis of ethyl 3-(4-cyanophenyl)-3-oxopropanoate

The acylated meldrum's acid obtained in step I (3.7 g) was refluxed with ethanol (50 ml) for about 20 hours, concentrated and purified by column chromatography over SiO₂ (100-200 mesh) using hexane/ethyl acetate gradient. The desired product eluted in 4-5% ethyl acetate in hexane (2.6 g).

EIMS m/z 218.37 [M+H]⁺

### Step III: Synthesis of ethyl 2-amino-4-(4-cyanophenyl)-1,3-thiazole-5-carboxylate

A solution of the ethyl 3-(4-cyanophenyl)-3-oxopropanoate (1 g, 4.68 mmol) in acetonitrile (10 ml) was treated with hydroxy-tosyloxyiodobenzene (2.16 g, 5.6 mmol, 1.2 equiv) and the mixture was stirred at room temperature (˜25° C.) for about 20 min and then heated at about 80° C. for about 2 hours. Thiourea (0.38 g, 5.6 mmol, 1.2 equiv) was added and the reaction mixture was heated at about 80° C. for 16 hours. After completion of the reaction, the acetonitrile was removed and the residue was treated with cold 5% aq. sodium bicarbonate solution and stirred for ˜20 min. The precipitate was filtered under vacuum and the residue washed 2-3 times with cold diethyl ether, dried under vacuum to afford the product (1.2 g).

EIMS m/z 274.25 [M+H]⁺

### Step IV: Synthesis of ethyl 2-bromo-4-(4-cyanophenyl)-1,3-thiazole-5-carboxylate

A mixture of ethyl 2-amino-4-(4-cyanophenyl)-1,3-thiazole-5-carboxylate (1 g. 3.66 mmol) and cupric bromide (0.41 g, 1.83 mmol) in acetonitrile was heated to about 65° C. and treated dropwise with isoamyl nitrite (0.66 ml) over a period of about 30 min. The reaction mixture was stirred at about 65° C. for another 1 hour, cooled and concentrated and the residue taken up in 1 N HCl solution and partitioned with ethyl acetate. The combined organics were washed with water, brine, dried and concentrated. The crude product was purified by column chromatography over SiO₂ (100-200) using hexane-ethyl acetate gradient. The product eluted in 4% ethyl acetate/hexane (250 mg).

EIMS m/z 338.1 [M+H]⁺

The following compounds were synthesized following a synthetic procedure similar to the one outlined above starting from the corresponding acids. The methyl and t-butyl esters were obtained by using methanol or ethanol as a solvent in step II.
Ethyl 4-benzyl-2-bromo-1,3-thiazole-5-carboxylate

EIMS m/z 326.14 [M+H]⁺
Ethyl 2-bromo-4-(phenoxymethyl)-1,3-thiazole-5-carboxylate

EIMS m/z 342.53 [M+H]⁺)
Ethyl 2-bromo-4-(cyclopentylmethyl)-1,3-thiazole-5-carboxylate

EIMS m/z 318.48 [M+H]⁺
Ethyl 2-bromo-4-(benzyloxymethyl)-1,3-thiazole-5-carboxylate

EIMS m/z 356.51 [M+H]⁺
Ethyl 2-bromo-4-(cyclopentylethyl)-1,3-thiazole-5-carboxylate

EIMS m/z 332.52 [M+H]⁺
Ethyl 2-bromo-4-(4-methoxy benzyl)-1,3-thiazole-5-carboxylate

EIMS m/z 356.51 [M+H]⁺
Methyl 2-bromo-4-(4-fluoro phenyl)-1,3-thiazole-5-carboxylate

EIMS m/z 318.48 [M+2]⁺
Methyl-2-bromo-4-(biphenyl-4-yl) 1,3-thiazole-5-carboxylate

EIMS m/z 376.53 [M+H]⁺
Methyl 2-bromo-4-(thien-3-yl)-1,3-thiazole-5-carboxylate
Ethyl 2-bromo-4-(3,4-di-fluoro benzyl)-1,3-thiazole-5-carboxylate

EIMS m/z 363.69 [M+H]⁺
Methyl-2-bromo-4-(1,3-benzodioxol-5-ylmethyl) 1,3-thiazole-5-carboxylate

EIMS m/z 371.68 [M+H]⁺
Ethyl-2-bromo-4-(2-chloro-4-fluoro-benzyl) 1,3-thiazole-5-carboxylate

EIMS m/z 379.67 [M+H]⁺
Ethyl-2-bromo-4-(2-bromo benzyl) 1,3-thiazole-5-carboxylate
Ethyl-2-bromo-4-(2-chloro phenyl) 1,3-thiazole-5-carboxylate
Ethyl-2-bromo-4-(3-chloro phenyl) 1,3-thiazole-5-carboxylate
Ethyl-2-bromo-4-(2-chloro benzyl) 1,3-thiazole-5-carboxylate
Ethyl-2-bromo-4-(2-naphthyl methyl) 1,3-thiazole-5-carboxylate

EIMS m/z 377.66 [M+H]⁺
Ethyl-2-bromo-4-(2-methylbenzyl) 1,3-thiazole-5-carboxylate

EIMS m/z 341.65 [M+H]⁺
Ethyl-2-bromo-4-(3-bromo benzyl) 1,3-thiazole-5-carboxylate

EIMS m/z 407.8 [M+2]⁺
Ethyl 2-bromo-4-(2-methoxy benzyl)-1,3-thiazole-5-carboxylate

EIMS m/z 379.67 [M+H]⁺
Ethyl 2-bromo-4-(1-naphthyl methyl)-1,3-thiazole-5-carboxylate

EIMS m/z 377.66 [M+H]⁺
Ethyl 2-bromo-4-(4-bromo-2-methyl phenyl)-1,3-thiazole-5-carboxylate

EIMS m/z 405.54 (M⁺)
Ethyl 2-bromo-4-(4-trifluoromethyl benzyl)-1,3-thiazole-5-carboxylate

EIMS m/z 395.66 [M+H]⁺
Ethyl 2-bromo-4-(2,4-di-chloro benzyl)-1,3-thiazole-5-carboxylate

EIMS m/z 396.00 [M+H]⁺
Ethyl 2-bromo-4-(4-ethoxy benzyl)-1,3-thiazole-5-carboxylate

EIMS m/z 370.12 (Nr)
Ethyl 4-(biphenyl-4-yl-methyl)-2-bromo-1,3-thiazole-5-carboxylate

EIMS ink 403.66 [M+H]⁺
Ethyl 4-(4-acetylphenyl)-2-bromo-1,3-thiazole-5-carboxylate

EIMS m/z 356.15 [M+2]⁺
Ethyl 2-bromo-4-(4-t-butyl phenyl)-1,3-thiazole-5-carboxylate

EIMS m/z 370.19 [M+2]⁺
Ethyl 2-bromo-4-(4-phenoxy-phenyl)-1,3-thiazole-5-carboxylate

EIMS m/z 406.08 [M+2]⁺
Ethyl 2-bromo-4-(3-methoxy benzyl)-1,3-thiazole-5-carboxylate

EIMS m/z 355.69 (M⁺)
Ethyl 2-bromo-4-(3-cyano phenyl)-1,3-thiazole-5-carboxylate

EIMS m/z 337.06 (M⁺)
Ethyl 2-bromo-4-[2-fluoro-5-trifluoromethyl-benzyl]-1,3-thiazole-5-carboxylate
Ethyl 2-bromo-4-(4-chloro benzyl)-1,3-thiazole-5-carboxylate

EIMS m/z 362.02 [M+2]⁺
Ethyl 2-bromo-4-(3-cyano benzyl)-1,3-thiazole-5-carboxylate
Ethyl 2-bromo-4-(2-ethoxy benzyl)-1,3-thiazole-5-carboxylate

EIMS m/z 372.14 [M+2]⁺
Ethyl 2-bromo-4-(4-acetyl amino-benzyl)-1,3-thiazole-5-carboxylate
Ethyl 2-bromo-4-(hydroxy methyl)-1,3-thiazole-5-carboxylate

EIMS m/z 266.11 [M+H]⁺
Ethyl 2-bromo-4-(4-ethoxy carbonyl-benzyl)-1,3-thiazole-5-carboxylate

EIMS m/z 400.09 [M+2]⁺
Ethyl 2-bromo-4-trifluoromethyl-1,3-thiazole-5-carboxylate
Ethyl 2-bromo-4-[2-(pyridin-4-yl)ethyl]-1,3-thiazole-5-carboxylate

EIMS m/z 329.02 [M+2]⁺
Ethyl 2-bromo-4-(3-nitro benzyl)-1,3-thiazole-5-carboxylate

EIMS m/z 373.10 [M+2]⁺
Ethyl 4-(5-acetylthiophene-2-yl)-2-bromo-1,3-thiazole-5-carboxylate

EIMS m/z 362.06 [M+2]⁺
Ethyl 2-bromo-4-(4-nitro benzyl)-1,3-thiazole-5-carboxylate

EIMS m/z 373.10 [M+2]⁺
Ethyl 2-bromo-4-(2,5-dimethoxy benzyl)-1,3-thiazole-5-carboxylate

EIMS m/z 388.13 [M+2]⁺
Ethyl 2-bromo-4-(3-trifluoromethyl benzyl)-1,3-thiazole-5-carboxylate

EIMS m/z 396.0 [M+2]⁺
Ethyl 2-bromo-4-(3,5-bis-trifluoromethyl benzyl)-1,3-thiazole-5-carboxylate

EIMS m/z 396.0 [M+2]⁺
Ethyl 2-bromo-4-(3-chloro phenyl)-1,3-thiazole-5-carboxylate
Ethyl 2-bromo-4-(3-chloro 4-fluoro phenyl)-1,3-thiazole-5-carboxylate
Ethyl 2-bromo-4-(4-butoxy benzyl)-1,3-thiazole-5-carboxylate
Methyl 2-bromo-4-(4-trifluoromethoxy phenyl)-1,3-thiazole-5-carboxylate
tert-butyl 2-bromo-4-[2-(methoxycarbonyl)benzyl]-1,3-thiazole-5-carboxylate
   Methyl 2-bromo-4-(2-methoxy-2-oxoethyl)-1,3-thiazole-5-carboxylate was prepared following a similar procedure starting from dimethyl acetone-1,3-dicarboxylate

EIMS m/z 296.16 [M+2]⁺

The following compounds were synthesized following a similar synthetic procedure starting from methyl 3-oxo-3-(pyridin-3-yl)propanoate Methyl 2-bromo-4-(3-pyridyl)-1,3-thiazole-5-carboxylate was synthesized following a similar synthetic procedure as above starting from methyl 3-oxo-3-(pyridin-3-yl)propanoate, which was prepared by following a similar synthetic procedure described in WO2008152418

EIMS m/z 313.0 [M+H]⁺

Methyl 2-bromo-4-(4-pyridyl)-1,3-thiazole-5-carboxylate was synthesized following a similar synthetic procedure as above starting from methyl 3-oxo-3-(pyridin-4-yl)propanoate, which was prepared by following a similar synthetic procedure described in WO2008152418

EIMS m/z 315.03 [M+2]⁺

### Synthesis of ethyl-2-bromo-4-propyl-1,3-thiazol-5-carboxylate

### Step I: Synthesis of ethyl 3-oxohexanoate

A ˜0° C. solution of meldrum's acid (5 g, 34.7 mmol), anhydrous dichloromethane (40 ml) was treated dropwise with pyridine (5.55 g, 69.4 mmol) and stirred for 15 min. Butryl chloride (4.06 g, 38 mmol, 1.1 equiv) was added dropwise while maintaining the temperature at about 0° C., and the reaction mixture was stirred at room temperature (˜25° C.) for about 1 hour. The reaction mixture was concentrated, added 6M HCl (100 ml) and extracted with ethyl acetate. The combined organics were dried over anhydrous sodium sulphate to afford acylated meldrum's acid (7 g). This was then refluxed in ethanol (50 ml) for about 20 hours. The reaction mixture was concentrated to dryness, taken up in ethyl acetate (200 ml) and washed with aq. sodium bicarbonate water, brine, dried (ann. Na₂SO₄) and concentrated to afford the crude compound that was purified by column chromatography over SiO₂ (100-200) and eluted in 2% EtOAc/Hexane (2 g).

EIMS m/z 159 [M+H]⁺

### Step II: Synthesis of ethyl 2-chloro-3-oxohexanoate

A solution of ethyl 3-oxohexanoate (2.0 g, 12.6 mmol) in dichloromethane was stirred at room temperature (˜25° C.) under inert atmosphere. Sulphuryl chloride (1.79 g, 13.2 mmol, 1.05 equiv.) was added dropwise and stirred at room temperature (˜25° C.) for about 90 min. The residue obtained after concentration was dissolved in ethyl acetate, dried over anhydrous sodium sulphate and concentrated over rotavapor to afford a viscous oil (1.5 g) that was taken as such to the next step (1.5 g).

EIMS m/z 193 [M+H]⁺

### Step III: Synthesis of ethyl-2-amino-4-propyl-1,3-thiazol-5-carboxylate

A mixture of ethyl 2-chloro-3-oxohexanoate (0.5 g, 2.5 mmol) and thiourea (0.197 g, 2.5 mmol) in acetonitrile was stirred at about 80° C. for about 20 hours. The reaction mixture was concentrated on a rotary evaporator and the residue was partitioned between water and dichloromethane. The combined organics were dried over sodium sulphate and concentrated. The crude compound was purified by column chromatography over neutralized silica gel (100-200) while eluting with 50% ethyl acetate/hexane to afford (0.15 g).

EIMS m/z 215 [M+H]⁺

### Step IV: Synthesis of ethyl-2-bromo-4-propyl-1,3-thiazol-5-carboxylate

Ethyl-2-amino-4-propylthiazol-5-carboxylate (0.15 g, 0.7 mmol) was dissolved in acetonitrile (40 ml) and treated with cupric bromide (0.1 g, 0.43 mmol) at room temperature (˜25° C.) and the reaction mixture was heated to about 65° C. A solution of isoamyl nitrite (0.16 g, 1.05 mmol) in acetonitrile was added dropwise at the same temperature. Upon complete addition the reaction mixture was cooled to room temperature (˜25° C.) and stirred for about 2 hours. Upon completion of reaction, the solvent was removed and the residue was diluted with dil. HCl (2M, 20 ml) and extracted with dichloromethane. The combined organics were washed with water, brine, dried over anhydrous sodium sulphate and concentrated. The crude compound was purified by using column chromatography SiO₂ (100-200) (0.05 g).

### EIMS m/z 280 [M+H]⁺

The following compounds are prepared following the similar route of synthesis
Ethyl-2-bromo-4-pentyl-1,3-thiazol-5-carboxylate

EIMS m/z 308.60 [M+2]⁺
Ethyl-2-bromo-4-phenyl-1,3-thiazol-5-carboxylate

EIMS m/z 312.48.60 (M⁺)
Ethyl-2-bromo-4-benzyl-1,3-thiazol-5-carboxylate

EIMS m/z 328 [M+2]±
Ethyl-2-bromo-4-pentyl-1,3-thiazol-5-carboxylate

EIMS m/z 308 [M+2]⁺
Ethyl-2-bromo-4-methoxymethyl-1,3-thiazol-5-carboxylate

EIMS m/z 282 [M+2]⁺
Diethyl 2-bromo-1,3-thiazole-4,5-dicarboxylate

EIMS m/z 282 [M+2]⁺
Ethyl-2-bromo-1,3-thiazol-4-acetate

EIMS m/z 252 [M+2]⁺

### Synthesis of Oxazoles

### Synthesis of ethyl-2-chloro-4-methyl-1,3-oxazol-5-carboxylate

### Step I: Synthesis of ethyl-2-amino-4-methyl-1,3-oxazol-5-carboxylate

A solution of the ethyl acetoacetate (1 g, 6.07 mmol) in acetonitrile (10 ml) was treated with hydroxy-tosyloxyiodobenzene (2.85 g, 7.28 mmol, 1.2 equiv) and the mixture was stirred at room temperature (˜25° C.) for about 20 min and then heated at about 80° C. for about 2 hours. Urea (0.43 g, 7.28 mmol, 1.2 equiv) was added and the reaction mixture was heated at about 80° C. for about 16 hours. After completion of the reaction, the acetonitrile was removed and the residue partitioned between 5% aq. Sodium bicarbonate solution and ethyl acetate. The combined organics were dried over sodium sulphate and concentrated. The crude compound was taken up in 6 N HCl solution and extracted with ether. The acidic portion was neutralized with solid Sodium bicarbonate under ice-cooled condition and the resultant solid was filtered under vacuum to afford the product (320 mg)

EIMS m/z 170.84 [M+H]⁺

### Step II: Synthesis of ethyl-2-chloro-4-methyl-1,3-oxazol-5-carboxylate

A solution of ethyl-2-amino-4-methyl-1,3-oxazol-5-carboxylate (0.25 g, 1.47 mmol) and tert-butyl nitrite (0.23 g, 2.21 mmol) was heated to about 65° C. and copper chloride (0.1 g, 0.735 mmol) was added in portions over a period of about 30 min. The reaction mixture was stirred at about 65° C. for about 1 hour and then at room temperature (˜25° C.) for about 16 hours. Upon completion of reaction, the solvent was removed and the residue was diluted with dil. HCl and extracted with dichloromethane. The combined organics were washed with water, brine, dried over anhydrous sodium sulphate and concentrated. The crude compound was purified by using column chromatography SiO₂(100-200) eluting with 3% EtOAc/hexane (130 mg)

EIMS m/z 190.24 [M+H]⁺
Ethyl-2-chloro-4-(4-chloro benzyl)-1,3-oxazol-5-carboxylate
Ethyl-2-chloro-4-(4-ethoxy benzyl)-1,3-oxazol-5-carboxylate

EIMS m/z 310.08 [M+H]⁺
Ethyl-2-chloro-4-(3-trifluoromethyl benzyl)-1,3-oxazol-5-carboxylate

### Example 1

### Synthesis of Ethyl 2[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxylate (Compound no. 153)

### Step I: Synthesis of tert-butyl (1R,5S,6s)-3-azabicyclo[3.1.0]hex-6-ylcarbamate

A solution of tert-butyl[(1R,5S,6s)-3-benzyl-3-azabicyclo[3.1.0]hex-6-yl]carbamate (8.64 g, 30 mmol) in methanol was treated with ammonium formate (10 g, 154 mmol) and 10% Pd/C (5 g, 50% w/w). This reaction mixture was stirred at about 60° C. for about 1 hour, cooled to ˜25° C. and filtered over celite. The filtrate was dried over anhydrous sodium sulfate and concentrated under vacuum. The residue was triturated with cold hexane and ether to afford the title compound (5.5 g)

EIMS m/z 199 [M+H]⁺

### Step II: Synthesis of Ethyl 2-{(1R,5S,6s)-6-[(tert-butoxycarbonyl)amino]-3-azabicyclo[3.1.0]hex-3-yl}-4-methyl-1,3-thiazole-5-carboxylate

To a solution of tert-butyl (1R,5S,6s)-3-azabicyclo[3.1.0]hex-6-ylcarbamate (1.5 g, 7.57 mmol) in anhydrous dimethylformamide, freshly activated potassium carbonate (1.25 g, 9.09 mmol) followed by ethyl-2-bromo-4-methyl-thiazole-5-carboxylate (1.8 g, 7.57 mmol) were added. The mixture was stirred at ˜25° C. for about 12 hours. The reaction was quenched with ice-cooled water and extracted with ethyl acetate. The combined organic layers were washed with water followed by brine, dried over anhydrous sodium carbonate and concentrated. The crude product thus obtained was purified using column chromatography to get the title compound (1 g).

EIMS m/z 368.19 [M+H]⁺

### Step III: Synthesis of Ethyl 2-[(1R,5S,6s)-6-amino-3-azabicyclo[3.1.0]hex-3-yl]-4-methyl-1,3-thiazole-5-carboxylate

Ethyl 2-{(1R,5S,6s)-6-[(tert-butoxycarbonyl)amino]-3-azabicyclo[3.1.0]hex-3-yl}-4-methyl-1,3-thiazole-5-carboxylate (0.4 g, 1.089 mmol) was treated with trifluoroacetic acid (5 ml, 20% solution in anhydrous dichloromethane) and stirred at ˜25° C. for about 16 hours. The resultant reaction mixture was concentrated under vacuum to obtain viscous oil, which was triturated with cold ether and filtered to obtain the title compound as a trifluoroacetate salt (245 mg).

EIMS m/z 268.19 [M+H]⁺

### Step IV: Synthesis of ethyl 2-[(1R,5S,6s)-6-{[(3,4-dichloro-5-methyl-1H-pyrrol-2-1)carbonyl]amino}-3-azabicyclo[3.1.0]hex-3-yl]-4-methyl-1,3-thiazole-5-carboxylate

To a solution of 3,4-dichloro-5-methyl-pyrrole-2-carboxylic acid (0.15 g, 0.76 mmol) in anhydrous dimethylformamide, N-hydroxybenzotriazole (0.124 g, 0.912 mmol), 1-ethyl-3-(3-dimethylaminopropyl)carbodiimide hydrochloride (0.174 g, 0.912 mmol), and N-methyl morpholine (0.32 ml, 2.28 mol) was added. To this reaction mixture, ethyl 2-[(1R,5S,6s)-6-amino-3-azabicyclo[3.1.0]hex-3-yl]-4-methyl-1,3-thiazole-5-carboxylate (0.29 g, 0.76 mmol) was added and was stirred at ˜25° C. for about 20 hours. The reaction was quenched with ice-cooled water and extracted with ethyl acetate. The combined organic layers were washed with 5% aq. sodium bicarbonate solution, water followed by brine, dried over anhydrous sodium sulfate and concentrated to obtain a solid residue. The obtained residue was purified using column chromatography to obtain the title compound (120 mg).

¹H NMR (400 MHz, CDCl₃+CD₃OD): δ 1.33-1.38 (q, 3H, J=7.12 Hz), 2.1-2.15 (m, 2H), 2.24 (s, 3H), 2.58-2.61 (m, 4H), 3.79-3.85 (m, 2H), 3.89-3.95 (m, 2H), 4.29-4.37 (q, 2H, J=7.12 Hz).

EIMS m/z 443.23 (M⁺)

The following compounds were prepared following similar synthetic route
Ethyl 2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 154)

EIMS m/z 428.95 (M⁺)
Methyl 2-chloro-6-[(1R,5S,6s)-6-{[(3,4-dichloro-5-methyl-1H-pyrrol-2-yl)carbonyl]amino}-3-azabicyclo[3.1.0]hex-3-yl]pyridine-4-carboxylate (Compound no. 155)

EIMS m/z 442.93 (M⁺)
Methyl 6-[(1R,5S,6s)-6-{[(3,4-dichloro-5-methyl-1H-pyrrol-2-yl)carbonyl]amino}-3-azabicyclo[3.1.0]hex-3-yl]pyridine-3-carboxylate (Compound no. 156)

EIMS m/z 409.26 (M⁺)

### Example 2

### Synthesis of 3,4-dichloro-N-[(1R,5S,6s)-3-(5-cyanopyridin-2-yl)-3-azabicyclo[3.1.0]hex-6-yl]-5-methyl-1H-pyrrole-2-carboxamide (Compound No. 5)

### Step I: Synthesis of tert-butyl[(1R,5S,6s)-3-(5-cyanopyridin-2-yl)-3-azabicyclo[3.1.0]hex-6-yl]carbamate

To a solution of tert-butyl (1R,5S,6s)-3-azabicyclo[3.1.0]hex-6-ylcarbamate (0.3 g, 1.5 mmol) in anhydrous dimethylformamide, freshly activated potassium carbonate (0.62 g, 4.5 mmol) followed by 6-chloropyridine-3-carbonitrile (0.31 g, 2.25 mmol) were added. The mixture was stirred at about 80° C. for about 16 hours. The reaction was quenched with ice-cooled water and extracted with ethyl acetate. The combined organic layers were washed with water followed by brine, dried over anhydrous sodium carbonate and concentrated. The crude product thus obtained was purified using column chromatography to get the title compound (100 mg).

EIMS m/z 301.11 [M+H]⁺

### Step II: Synthesis of 6-[(1R,5S,6s)-6-amino-3-azabicyclo[3.1.0]hex-3-yl]pyridine-3-carbonitrile

tert-Butyl[(1R,5S,6s)-3-(5-cyanopyridin-2-yl)-3-azabicyclo[3.1.0]hex-6-yl]carbamate

(0.5 g, 1.57 mmol) was treated with trifluoroacetic acid (10 ml, 20% solution in anhydrous dichloromethane) and stirred at ˜25° C. for about 16 hours. The resultant reaction mixture was concentrated under vacuum to obtain viscous oil, which was triturated with cold ether and filtered to obtain the title compound as a trifluoroacetate salt (450 mg).

EIMS m/z 201.15 [M+H]⁺

### Step III: Synthesis of 3,4-dichloro-N-[(1R,5S,6s)-3-(5-cyanopyridin-2-yl)-3-azabicyclo[3.1.0]hex-6-yl]-5-methyl-1H-pyrrole-2-carboxamide

To a solution of 3,4-dichloro-5-methyl-pyrrole-2-carboxylic acid (124 mg, 0.64 mmol) in anhydrous N,N-dimethylformamide (3 ml), N-hydroxybenzotriazole (0.10 g, 0.76 mmol), 1-ethyl-3-(3-dimethylaminopropyl)carbodiimide hydrochloride (0.14 g, 0.76 mmol) and N,N-diisopropyl ethylamine (0.16 g, 1.28 mmol) was added. To this reaction mixture, a N,N-dimethylformamide (2 ml), 6-[(1R,5S,6s)-6-amino-3-azabicyclo[3.1.0]hex-3-yl]pyridine-3-carbonitrile trifluoroacetate salt (0.2 g, 0.64 mmol) previously treated with N,N-diisopropyl ethylamine (0.083 g, 0.64 mmol) was added and the reaction mixture was stirred at ˜25° C. for about 16 hours. The reaction was quenched with ice-cooled water and extracted with ethyl acetate. The combined organic layers were washed with 5% aq. sodium bicarbonate solution; water followed by brine, dried over anhydrous sodium sulfate and concentrated to obtain a solid residue that after triturating with ether afforded the title compound (100 mg).

¹H NMR (400 MHz, CD₃OD): δ 2.06-2.1 (m, 2H), 2.27 (s, 3H), 2.50-2.55 (m, 1H), 3.55-3.62 (m, 2H), 3.85-3.92 (m, 2H), 6.56 (d, 1H, J=9 Hz), 7.72 (d, 1H, J=8.72 Hz), 8.36 (s, 1H).

EIMS m/z 376.06 (M⁺)

The following compounds were prepared following similar synthetic route:
2-[(1R,5S,6s)-6-{[(4-chloro-5-methyl-1H-pyrrol-2-yl)carbonyl]amino}-3-azabicyclo[3.1.0]hex-3-yl]pyridine-3-carboxamide (Compound No. 3)

EIMS m/z 360.09 (M⁺)
2-[(1R,5S,6s)-6-{[(3,4-dichloro-5-methyl-1H-pyrrol-2-yl)carbonyl]amino}-3-azabicyclo[3.1.0]hex-3-yl]pyridine-3-carboxamide (Compound No. 4)

EIMS m/z 394.05 (M⁺)

### Example 3

### Ethyl 3-[(1R,5S,6s)-6-{[(3,4-dichloro-5-methyl-1H-pyrrol-2-yl)carbonyl]amino}-3-azabicyclo[3.1.0]hex-3-yl]benzoate

### Step I: Synthesis of di-tert-butyl (1R,5S,6s)-3-azabicyclo[3.1.0]hex-6-ylimidodicarbonate

A solution of di-tert-butyl[(1R,5S,6s)-3-benzyl-3-azabicyclo[3.1.0]hex-6-yl]imidodicarbonate (12 g, 30 mmol) in methanol was treated with ammonium formate (10 g, 154 mmol) and 10% Pd/C (5 g, 50% w/w). This reaction mixture was stirred at about 60° C. for about 1 hour, cooled to ˜25° C. and filtered over celite. The filtrate was dried over anhydrous sodium sulfate and concentrated under vacuum. The residue was triturated with cold hexane and ether to afford the title compound (8 g)

EIMS m/z 299.44 [M+H]⁺

### Step II: Synthesis of ethyl 3-{(1R,5S,6s)-6-[bis(tert-butoxycarbonyl)amino]-3-azabicyclo[3.1.0]hex-3-yl}benzoate

A mixture of di-tert-butyl (1R,5S,6s)-3-azabicyclo[3.1.0]hex-6-ylimidodicarbonate (0.1 g, 0.33 mmol), cesium carbonate (0.13 g, 0.405 mmol), ethyl-3-bromobenzoate (0.064 g, 0.27 mmol) and 2,2′-bis(diphenylphosphino)-1,1′-binaphthyl (0.02 g, 0.027 mmol) in dry toluene was degassed and treated with palladium(II) acetate (0.003 g 0.0315 mmol). The reaction mixture was heated at about 80° C. for about 16 hours. The reaction mixture was concentrated under vacuum, diluted with ethyl acetate, washed with water, brine, dried over anhydrous sodium sulfate and concentrated under vacuum. The residue was purified by column chromatography over silicon dioxide (100-200) using hexane/ethyl acetate system as eluent to afford the title compound (100 mg).

EIMS m/z 447.29 [M+H]⁺

### Step III: Synthesis of Ethyl 3-[(1R,5S,6s)-6-amino-3-azabicyclo[3.1.0]hex-3-yl]benzoate

Ethyl 3-{(1R,5S,6s)-6-[bis(tert-butoxycarbonyl)amino]-3-azabicyclo[3.1.0]hex-3-yl}benzoate (0.24 g, 0.56 mmol) was treated with trifluoroacetic acid (5 ml, 20% solution in anhydrous dichloromethane) and stirred at ˜25° C. for about 16 hours. The resultant reaction mixture was concentrated under vacuum to obtain viscous oil, which was triturated with cold ether and filtered to obtain the title compound as a trifluoroacetate salt (200 mg).

EIMS m/z 247.5 [M+H]⁺

### Step IV: Synthesis of Ethyl 3-[(1R,5S,6s)-6-{[(3,4-dichloro-5-methyl-1H-pyrrol-2-yl)carbonyl]amino}-3-azabicyclo[3.1.0]hex-3-yl]benzoate

To a solution of 3,4-dichloro-5-methyl-pyrrole-2-carboxylic acid (75 mg, 0.38 mmol) in anhydrous dimethylformamide, N-hydroxybenzotriazole (0.06 g, 0.46 mmol), 1-ethyl-3-(3-dimethylaminopropyl) carbodiimide hydrochloride (0.09 g, 0.46 mmol) and N,N-Diisopropyl ethylamine (0.15 g 1.15 mmol) was added. To this reaction mixture, ethyl 3-[(1R,5S,6s)-6-amino-3-azabicyclo[3.1.0]hex-3-yl]benzoate trifluoroacetate salt (0.14 g, 0.38 mmol) was added and the reaction mixture was stirred at ˜25° C. for about 16 hours. The reaction was quenched with ice-cooled water and extracted with ethyl acetate. The combined organic layers were washed with 5% aq. sodium bicarbonate solution, water followed by brine, dried over anhydrous sodium sulfate and concentrated to obtain a solid residue. The obtained residue was purified using column chromatography to obtain the title compound (60 mg).

EIMS m/z 422.3 [M+H]⁺

¹H NMR (CD₃OD, 400 MHz) δ 1.35-1.39 (t, 3H, 7.08 Hz), 1.96-2.01 (m, 2H), 2.23 (s, 3h), 2.58-2.63 (m, 1H), 3.75-3.78 (m, 2H), 4.31-4.36 (q, 2H, 7.08 Hz), 6.81-6.83 (m, 1H), 7.21-7.32 (m, 3H).

### Example 4

### Ethyl 2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-4-carboxylate trifluoroacetate salt

### Step I: Synthesis of 1-[(1R,5S,6s)-6-amino-3-azabicyclo[3.1.0]hex-3-yl]-2,2,2-trifluoroethanone

tert-Butyl[(1R,5S,6s)-3-(trifluoroacetyl)-3-azabicyclo[3.1.0]hex-6-yl]carbamate (6 g, 20.4 mmol) was treated with trifluoroacetic acid (50 ml, 20% solution in anhydrous dichloromethane) and stirred at ˜25° C. for about 16 hours. The resultant reaction mixture was concentrated under vacuum to obtain viscous oil, which was triturated with cold ether and filtered to obtain the title compound as a trifluoroacetate salt (6.4 g).

EIMS m/z 195.41 [M+H]⁺

### Step II: Synthesis of 3,4-dichloro-5-methyl-N-[(1R,5S,6s)-3-(trifluoroacetyl)-3-azabicyclo[3.1.0]hex-6-yl]-1H-pyrrole-2-carboxamide

To a solution of 3,4-dichloro-5-methyl-pyrrole-2-carboxylic acid (4 g, 2.04 mmol) in anhydrous dimethylformamide (20 ml), N-hydroxybenzotriazole (3.36 g, 24.9 mmol), 1-ethyl-3-(3-dimethylamino-propyl) carbodiimide hydrochloride (4.76 g, 0.24.9 mmol) and N,N-diisopropyl ethylamine (5.36 g, 41.6 mmol) was added. To this reaction mixture, dimethylformamide (5 ml), 1-[(1R,5S,6s)-6-amino-3-azabicyclo[3.1.0]hex-3-yl]-2,2,2-trifluoroethanone trifluoroacetate salt (0.14 g, 0.38 mmol) previously treated with N,N-diisopropyl ethylamine (2.68 g, 20.8 mmol) was added and the reaction mixture was stirred at ˜25° C. for about 16 hours. The reaction was quenched with ice-cooled water and extracted with ethyl acetate. The combined organic layers were washed with 5% aq. sodium bicarbonate solution; water followed by brine, dried over anhydrous sodium sulfate and concentrated to obtain a solid residue that after triturating with ether afforded the title compound (2.7 g).

EIMS m/z 370.61 [M+H]⁺

### Step III: Synthesis of N-[(1R,5S,6s)-3-azabicyclo[3.1.0]hex-6-yl]-3,4-dichloro-5-methyl-1H-pyrrole-2-carboxamide

A solution of 3,4-dichloro-5-methyl-N-[(1R,5S,6s)-3-(trifluoroacetyl)-3-azabicyclo[3.1.0]hex-6-yl]-1H-pyrrole-2-carboxamide (2.7 g, 7.2 mmol) in methanol (15 ml) was treated with saturated aqueous potassium carbonate solution (15 ml) and the reaction mixture was stirred at room temperature (˜25° C.) for about 6 hours. The reaction mixture was concentrated to remove methanol, and the resultant mixture was filtered under vacuum. The residue was washed with water, dried under vacuum to afford the title compound (1.8 g).

EIMS m/z 274.55 [M+H]⁺

### Step IV: Synthesis of Ethyl 2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-4-carboxylate

To a solution of N-[(1R,5S,6s)-3-azabicyclo[3.1.0]hex-6-yl]-3,4-dichloro-5-methyl-1H-pyrrole-2-carboxamide (0.15 g, 0.54 mmol) in anhydrous dimethylformamide (3 ml), freshly activated potassium carbonate (0.09 g, 0.65 mmol) followed by ethyl-2-bromo-thiazole-4-carboxylate (0.1 g, 0.54 mmol) were added. The mixture was stirred at about 70° C. for about 16 hours. The reaction was quenched with ice-cooled water and extracted with ethyl acetate. The combined organic layers were washed with water followed by brine, dried over anhydrous sodium carbonate and concentrated. The crude product thus obtained was purified using column chromatography to get the title compound (40 mg).

NMR ¹H (400 MHz, CDCl₃+CD₃OD): δ 1.35-1.39 (t, 3H, J=7.2 Hz), 2.01-2.1 (m, 2H), 2.24 (s, 3H), 2.59-2.62 (m, 1H), 3.64-3.66 (m, 2H), 3.84-3.87 (m, 2H), 7.42 (s, 1H).

EIMS m/z 429.52 (M⁺)

Following compounds were prepared by following the similar route of synthesis as above
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(methoxymethyl)-1,3-thiazole-5-carboxylate (Compound no. 161)

EIMS m/z 473.3 (M⁺)
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(phenoxymethyl)-1,3-thiazole-5-carboxylate (Compound no. 162)

MS m/z 535.4 (M⁺)
Ethyl 4-(cyclopentylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 163)

EIMS m/z 511.4 (M⁺)
Ethyl 4-[(benzyloxy)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 164)

EIMS m/z 549.46 (M⁺)
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(3-fluorobenzyl)-1,3-thiazole-5-carboxylate (Compound no. 165)

EIMS m/z 537.4 (M⁺)
Ethyl 4-(2-cyclopentylethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 166)

EIMS m/z 525.49 (M⁺)
Ethyl 4-cyclohexyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 167)

EIMS m/z 511.4 (M⁺)
Ethyl 4-(2-chlorophenyl)-2-[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylate (Compound no. 168)

EIMS m/z 539.86 (M⁺)
Ethyl 4-(4-bromo-2-methylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 169)

EIMS m/z 598.3 (M⁺)
Ethyl 4-(4-butoxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 172)

EIMS m/z 591.4 (M⁺)
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(3-methoxybenzyl)-1,3-thiazole-5-carboxylate (Compound no. 173)

EIMS m/z 549.4 (M⁺)
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(4-ethoxybenzyl)-1,3-thiazole-5-carboxylate (Compound no. 174)

MS m/z 563.4 (M⁺)
Ethyl 4-(biphenyl-4-ylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 175)

EIMS m/z 595.13 (M⁺)
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(2,5-dimethoxybenzyl)-1,3-thiazole-5-carboxylate (Compound no. 176)

EIMS m/z 579 (M⁺)
tert-Butyl 2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxylate (Compound no. 177)

EIMS m/z 471 (M⁺)
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(pyridin-4-yl)-1,3-thiazole-5-carboxylate (Compound no. 178)

EIMS m/z 506 (M⁺)
{2-[(1R,5S,6s)-6-{[(3,4-d i chloro-5-methyl-1H-pyrrol-2-yl)carbonyl]amino}-3-azabicyclo[3.1.0]hex-3-yl]-5-(methoxycarbonyl)-1,3-thiazol-4-yl}acetic acid (Compound no. 179)

EIMS m/z 473.15 (M⁺)

### Example 5

### Synthesis of N-[(1R,5S,6s)-3-(1,3-benzothiazol-2-0)-3-azabicyclo[3.1.0]hex-6-yl]-3,4-dichloro-5-methyl-1H-pyrrole-2-carboxamide (Compound No. 32)

To a solution of N-[(1R,5S,6s)-3-azabicyclo[3.1.0]hex-6-yl]-3,4-dichloro-5-methyl-1H-pyrrole-2-carboxamide obtained from Step III Example 4 (0.2 g, 0.72 mmol) in anhydrous dimethylformamide (3 ml), freshly activated potassium carbonate (0.15 g, 1.0 mmol) followed by 2-chloro-benzthiazole (0.11 g, 0.6 mmol) were added. The mixture was stirred at about 70° C. for about 16 hours. The reaction was quenched with ice-cooled water and extracted with ethyl acetate. The combined organic layers were washed with water followed by brine, dried over anhydrous sodium carbonate and concentrated. The crude product thus obtained was purified after trituration with cold diethyl ether to get the title compound (70 mg).

¹H NMR (400 MHz, CDCl₃+CD₃OD): 2.04-2.1 (m, 2H), 2.25 (s, 3H), 2.55-2.66 (m, 1H), 3.73-3.79 (m, 2H), 3.92-3.96 (m, 2H), 7.09-7.11 (m, 1H), 7.28-7.32 (m, 1H), 7.53-7.58 (d, 1H, 7.92 Hz), 7.59-7.66 (d, 11-1, 7.16 Hz).

EIMS m/z 407.52, 409.49 (M⁺)

The following compound is made by following the similar route of synthesis
3,4-dichloro-5-methyl-N-{(1R,5S,6s)-3-[4-(naphthalen-2-yl-methyl)-1,3-thiazol-2-yl]-3-azabicyclo[3.1.0]hex-6-yl}-1H-pyrrole-2-carboxamide (Compound No. 70)

EIMS m/z 496.73, 498.68 (M⁺)

### Example 6

### Synthesis of 2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-4-carboxylic acid (Compound No. 23)

A mixture of ethyl 2-[(1R,5S)-6-{[(3,4-dichloro-5-methyl-1H-pyrrol-2-yl)carbonyl]amino}-3-azabicyclo[3.1.0]hex-3-yl]-1,3-thiazole-4-carboxylate (0.07 g, 0.163 mmol) and hydrated lithium hydroxide (0.24 g, 5.7 mmol) in tetrahydrofuran:water (1:1, 4 ml) was heated at about 70° C. for about 20 hours. The reaction mixture was concentrated under vacuum. The aqueous layer was acidified with 2N HCl under ice-cooled conditions, filtered. The residue was trirurated with cold diethyl ether to afford the title compound (30 mg).

EIMS m/z 401.4 (M⁺)

¹H NMR (400 MHz, CDCl₃+CD₃OD): δ 2.06 (m, 2H), 2.26 (s, 3H), 2.62-2.63 (m, 1H), 3.70-3.73 (m, 2H), 3.87-3.90 (m, 2H), 7.48 (s, 1H).

Following compounds are prepared by following the similar route of synthesis as mentioned using upto 30 equiv of base. The reactions temperature varied from 70° C. (conventional heating) to 100-150° C. under microwave irradiation.
tert-Butyl 2-[(1R,5S,6s)-6-{[(3,4-dichloro-5-methyl-1H-pyrrol-2-yl)carbonyl]amino}-3-azabicyclo[3.1.0]hex-3-yl]4-[2-(methoxycarbonyl)benzyl]-1,3-thiazole-5-carboxylate (Compound no. 160)

EIMS m/z 605.16 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(methoxymethyl)-1,3-thiazole-5-carboxylic acid (Compound No. 24),

EIMS m/z 445.59 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-pentyl-1,3-thiazole-5-carboxylic acid (Compound No. 25),

EIMS m/z 471.62 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-phenyl-1,3-thiazole-5-carboxylic acid (Compound No. 26),

EIMS m/z 477.53 (M⁻)
2-[(1R,5S,6s)-6-{[(3,4-dichloro-5-methyl-1H-pyrrol-2-yl)carbonyl]amino}-3-azabicyclo[3.1.0]hex-3-yl]4-[2-(methoxycarbonyl)benzyl]-1,3-thiazole-5-carboxylic acid (Compound No. 33)

EIMS m/z 547.23 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-(phenoxymethyl)-1,3-thiazole-5-carboxylic acid (Compound No. 34)

EIMS m/z 507.73 (M⁻)
4-(2-chloro-6-fluorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 35)

EIMS m/z 541.70 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-(2-phenylethyl)-1,3-thiazole-5-carboxylic acid (Compound No. 36)

EIMS m/z 505.78 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-(4-fluorobenzyl)-1,3-thiazole-5-carboxylic acid (Compound No. 37)

EIMS m/z 509.74 (M⁺)
4-(3-chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 38)

EIMS m/z 525.73 (M⁻)
4-(cyclopentylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 39)

EIMS m/z 481.73 (M⁻)
4-[(benzyloxy)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 40)

EIMS m/z 521.83 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(3-fluorobenzyl)-1,3-thiazole-5-carboxylic acid (Compound No. 41)

EIMS m/z 509.74 (M⁺)
4-cyclohexyl-2-[(1R,5S,6 s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 42)

EIMS m/z 483.7 (M⁺)
4-(2-cyclopentylethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 43)

EIMS m/z 497.3 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(4-methoxybenzyl)-1,3-thiazole-5-carboxylic acid (Compound No. 44)

EIMS m/z 521.77 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(4-fluorophenyl)-1,3-thiazole-5-carboxylic acid (Compound No. 45)

EIMS m/z 495.63 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(3-fluorophenyl)-1,3-thiazole-5-carboxylic acid (Compound No. 46)

EIMS m/z 495.63 (M⁺)
4-(biphenyl-4-yl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 47)

EIMS m/z 553.88 (M⁻)
4-butyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 48)

EIMS m/z 455.73 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-(naphthalen-2-yl)-1,3-thiazole-5-carboxylic acid (Compound No. 49)

EIMS m/z 527.68 (M⁺)
4-cyclopentyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 50)

EIMS m/z 467.69 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(3,5-dimethoxyphenyl)-1,3-thiazole-5-carboxylic acid (Compound No. 51)

EIMS m/z 537.69 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-[4-(trifluoromethoxy)phenyl]-1,3-thiazole-5-carboxylic acid (Compound No. 52)

EIMS m/z 561.74 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(thiophen-2-yl)-1,3-thiazole-5-carboxylic acid (Compound No. 53)

EIMS m/z 483.54 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(3,4-difluorobenzyl)-1,3-thiazole-5-carboxylic acid (Compound No. 54)

EIMS m/z 526.7 (M⁺)
4-(1,3-benzodioxol-5-ylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 55)

EIMS m/z 534.7 (M⁺)
4-(2-chloro-4-fluorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 56)

EIMS m/z 542.63 (M⁺)
4-(2-bromobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 57)

EIMS m/z 569.64 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(naphthalen-2-ylmethyl)-1,3-thiazole-5-carboxylic acid (Compound No. 58)

EIMS m/z 540.92 (M⁺)
4-(2-chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 59)

EIMS m/z 524.62 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(2-methylbenzyl)-1,3-thiazole-5-carboxylic acid (Compound No. 60)

EIMS m/z 504.59 (M⁺)
4-(3-bromobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 61)

EIMS m/z 569.6 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(2-methoxybenzyl)-1,3-thiazole-5-carboxylic acid (Compound No. 62)

EIMS m/z 520.77 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(naphthalen-1-ylmethyl)-1,3-thiazole-5-carboxylic acid (Compound No. 63)

EIMS m/z 540.67 (M⁺)
4-(2-chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 64)

EIMS m/z 511 (M⁺)
4-(3-chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 65)

EIMS m/z 510.67 (M⁺)
4-(4-bromo-2-methylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 66)

EIMS m/z 570.66 (M⁺)
4-(3-chloro-4-fluorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 67)

EIMS m/z 528.74 (M⁺)
4-(4-chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 68)

EIMS m/z 513.10 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-[4-(trifluoromethyl)benzyl]-1,3-thiazole-5-carboxylic acid (Compound No. 69)

EIMS m/z 558.8 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(4-phenoxyphenyl)-1,3-thiazole-5-carboxylic acid (Compound No. 81)

EIMS m/z 569.24 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-[3-(trifluoromethyl)benzyl]-1,3-oxazole-5-carboxylic acid (Compound No. 96)

EIMS m/z 543.13 (M⁺)
4-(4-chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-oxazole-5-carboxylic acid (Compound No. 97)

EIMS m/z 509.20 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(4-ethoxybenzyl)-1,3-oxazole-5-carboxylic acid (Compound No. 98)

EIMS m/z 518.96 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(pyridin-3-yl)-1,3-thiazole-5-carboxylic acid (Compound No. 99)

EIMS m/z 476.29 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(pyridin-4-yl)-1,3-thiazole-5-carboxylic acid (Compound No. 100)

EIMS m/z 476.29 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(trifluoromethyl)-1,3-thiazole-5-carboxylic acid (Compound no. 101),

EIMS m/z 506.27 (M⁺)
2-[(1R,5S {[(3,4-dichloro-5-methyl-1H-pyrrol-2-yl)carbonyl]amino}-3-azabicyclo[3.1.0]hex-3-yl]4-[2-(pyridin-4-yl)ethyl]-1,3-thiazole-5-carboxylic acid (Compound no. 102),

EIMS m/z 506.17 (M⁺)
ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(hydroxymethyl)-1,3-thiazole-5-carboxylate (Compound no. 104),

EIMS m/z 461.1 (M⁺)
4-(5-acetylthiophen-2-yl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 105),

EIMS m/z 525.32 (M⁺)
2-[(1R,5S,6s)-6-{[(3,4-dichloro-5-methyl-1H-pyrrol-2-yl)carbonyl]amino})-3-azabicyclo[3.1.0]hex-3-yl]-4-[3-(trifluoromethyl)benzyl]-1,3-thiazole-5-carboxylic acid

Lithium salt (Compound no. 108),

EIMS m/z 551.32 (M⁻)
4-(2-Carboxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 111),

EIMS m/z 535.23, (M⁻)
2-({5-(tert-butoxycarbonyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazol-4-yl}methyl)benzoic acid (Compound no. 112),

EIMS m/z 535.23, (M⁻)

### Example 7

### Synthesis of 2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxylic acid Lithium salt (Compound No. 1)

A solution of ethyl 2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxylate (0.12 g, 0.27 mmol) in tetrahydrofuran:water (1:1, 3 ml) was mixed with an aqueous solution of lithium hydroxide (0.23 g, 5.4 mmol). The reaction mixture was stirred at about 70° C. for about 20 hours. On completion, the solvent was removed under reduced pressure. The obtained residue was diluted with water, acidified with cold 2N HCl, and extracted with ethyl acetate. The combined organic layers were washed water followed by brine, dried over anhydrous sodium sulfate and concentrated. The solid residue thus obtained was triturated with cold ether to afford the acid (0.07 g). A solution of 2-[(1R,5S,6s)-6-{[(3,4-dichloro-5-methyl-1H-pyrrol-2-yl)carbonyl]amino}-3-azabicyclo[3.1.0]hex-3-yl]-4-methylthiazole-5-carboxylic acid (0.054 g, 0.13 mmol) in tetrahydrofuran (2 ml) was stirred with LiOH. H₂O (0.005 g, 0.12 mmol) at room temperature for ˜1 hour, concentrated and dried under vacuum to afford the product as a lithium salt (56 mg).

NMR ¹H (400 MHz, CDCl₃+CD₃OD): δ 2.01-2.1 (m, 2H), 2.24 (s, 3H), 2.52 (s, 3H), 2.52-2.60 (m, 1H), 3.58-3.60 (m, 2H), 3.77-3.80 (m, 2H) EIMS m/z 414.99 (M⁻)

The following compounds were prepared by a similar procedure
2-[(1R,5S,6s)-6-{[(3,4-dichloro-5-methyl-1H-pyrrol-2-yl)carbonyl]amino}-3-azabicyclo [3.1.0]hex-3-yl]-1,3-thiazole-5-carboxylic acid Lithium salt (Compound No. 2)

EIMS m/z 400.96 (M⁻)
2-[(1R,5S,6s)-6-{[(4-chloro-5-methyl-1H-pyrrol-2-yl)carbonyl]amino}-3-azabicyclo[3.1.0]hex-3-yl]-4-methyl-1,3-thiazole-5-carboxylic acid Lithium salt (Compound No. 6)

EIMS m/z 381.03 (M⁻)
2-chloro-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylic acid Lithium salt (Compound No. 7)

EIMS m/z 430.56 (M⁻)
2-chloro-6-[(1R,5S,6s)-6-{[(3,4-dichloro-5-methyl-1H-pyrrol-2-yl)carbonyl]amino}-3-azabicyclo[3.1.0]hex-3-yl]pyridine-4-carboxylic acid Lithium salt (Compound No. 8)

EIMS m/z 428.95 (M⁻)
6-[(1R,5S,6s)-6-{[(3,4-dichloro-5-methyl-1H-pyrrol-2-yl)carbonyl]amino}-3-azabicyclo[3.1.0]hex-3-yl]pyridine-2-carboxylic acid Lithium salt (Compound No. 9)

EIMS m/z 394.96 (M⁻)
6-[(1R,5S,6s)-6-{[(3,4-dichloro-5-methyl-1H-pyrrol-2-yl)carbonyl]amino}-3-azabicyclo[3.1.0]hex-3-yl]pyridine-3-carboxylic acid Lithium salt (Compound No. 10)

EIMS m/z 395.02 (M⁻)
{2-[(1R,5S,6s)-6-{[(3,4-dichloro-5-methyl-1H-pyrrol-2-yl)carbonyl]amino}-3-azabicyclo[3.1.0]hex-3-yl]-1,3-thiazol-4-yl}acetic acid Lithium salt (Compound No. 12)

EIMS m/z 415.27 (M⁻)
5-[(1R,5S,6s)-6-{[(3,4-dichloro-5-methyl-1H-pyrrol-2-yl)carbonyl]amino}-3-azabicyclo[3.1.0]hex-3-yl]furan-2-carboxylic acid Lithium salt (Compound No. 13)

EIMS m/z 341.31 (M⁺—CO₂)
5-[(1R,5S,6s)-6-{[(3,4-dichloro-5-methyl-1H-pyrrol-2-yl)carbonyl]amino}-3-azabicyclo[3.1.0]hex-3-yl]thiophene-2-carboxylic acid Lithium salt (Compound No. 14)

EIMS m/z 400.24 (M⁺)
6-[(1R,5S,6s)-6-{[(3,4-dichloro-5-methyl-1H-pyrrol-2-yl)carbonyl]amino}-3-azabicyclo[3.1.0]hex-3-yl]naphthalene-2-carboxylic acid Lithium salt (Compound No. 15),

EIMS m/z 444.39 (M⁺)
{4-[(1R,5S,6s)-6-{[(3,4-dichloro-5-methyl-1H-pyrrol-2-yl)carbonyl]amino}-3-azabicyclo[3.1.0]hex-3-yl]phenyl}acetic acid Lithium salt (Compound No. 17),

EIMS m/z 408.16 (M⁺)
3-[(1R,5S,6s)-6-{[(3,4-dichloro-5-methyl-1H-pyrrol-2-yl)carbonyl]amino}-3-azabicyclo [3.1.0]hex-3-yl]benzoic acid Lithium salt (Compound No. 18),

EIMS m/z 394.2 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-propyl-1,3-thiazole-5-carboxylic acid Lithium salt (Compound No. 20),

EIMS m/z 443.71 (m⁺)
4-benzyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 21),

EIMS m/z 491.74 (M⁺)
4-(2,4-dichlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 71)

EIMS m/z 561.01 (M⁺)
4-(4-butoxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 72)

EIMS m/z 563.41 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-(4-ethoxybenzyl)-1,3-thiazole-5-carboxylic acid Lithium salt (Compound No. 73)

EIMS m/z 535.54, (M)
4-(biphenyl-4-yl-methyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 74)

EIMS m/z 568.07 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-(5-methylthiophen-2-yl)-1,3-thiazole-5-carboxylic acid Lithium salt (Compound No. 75)

EIMS m/z 5497.50 (M⁻)
4-[3,5-bis(trifluoromethyl)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 76)

EIMS m/z 627.3 (M⁻)
4-(4-acetylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 79)

ELMS m/z 519.12 (M⁻)
4-(4-tert-butylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 80)

EIMS m/z 531.35, (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-(3-methoxybenzyl)-1,3-thiazole-5-carboxylic acid Lithium salt (Compound No. 82)

EIMS m/z 521.44 (M⁻)
4-(4-carboxyphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 83)

EIMS m/z 523.58 (M⁺)
4-(3-carboxyphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 84)

EIMS m/z 521.05 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4,2-fluoro-5-(trifluoromethyl)benzyl]-1,3-thiazole-5-carboxylic acid Lithium salt (Compound No. 85)

EIMS m/z 577.10 (M⁻)
4-(4-chlorobenzyl)-2-{[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 86)

EIMS m/z 525.17 (M⁻)
4-(3-carboxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 87)

EIMS m/z 535.23 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-(2-ethoxybenzyl)-1,3-thiazole-5-carboxylic acid Lithium salt (Compound No. 88)

EIMS m/z 533.26 (M⁻)
4-(4-carboxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 89)

EIMS m/z 535.17 (M⁻)
4-[4-(acetylamino)benzyl]-2-{[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 90)

EIMS m/z 548.02 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-(hydroxymethyl)-1,3-thiazole-5-carboxylic acid Lithium salt (Compound No. 91)

EIMS m/z 431.07[M+H]⁺
2-[(1R,5S,6s)-6-{[(3,4-dichloro-5-methyl-1H-pyrrol-2-yl)carbonyl]amino}-3-azabicyclo[3.1.0]hex-3-yl]-4-[4-(ethylcarbamoyl)benzyl]-1,3-thiazole-5-carboxylic acid Lithium salt (Compound No. 92)

EIMS m/z 560.45 [M+H]⁺
2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-oxazole-5-carboxylic acid Lithium salt (Compound No. 95)

EIMS m/z, 401.11 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-{3-[(ethylcarbamoyl)amino]benzyl}-1,3-thiazole-5-carboxylic acid Lithium salt (Compound no. 109),

EIMS m/z 559.36 (M⁻)

The following compound was prepared using the above mentioned procedure using 1.0 equiv sodium hydroxide for the salt formation.
4-(biphenyl-4-yl-methyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Sodium salt (Compound No. 74a)

EIMS m/z 568.07 (M⁺)

The following compound was prepared using the above mentioned procedure using 2 equiv sodium hydroxide for the salt formation.
2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-4,5-dicarboxylic acid di-sodium salt (Compound No. 27).

EIMS m/z 445.65 (M⁺)

### Example 8

### Synthesis of 6-[(1R,5S,6s)-6-{[(3,4-dichloro-5-methyl-1H-pyrrol-2-yl)carbonyl]amino}-3-azabicyclo[3.1.0]hex-3-yl]-2-(morpholin-4-yl)pyrimidine-4-carboxylic acid Lithium salt (Compound No. 11)

### Step I: Synthesis of Methyl 6-[(1R,5S,6s)-6-{[(3,4-dichloro-5-methyl-1H-pyrrol-2-yl)carbonyl]amino}-3-azabicyclo[3.1.0]hex-3-yl]-2-(morpholin-4-yl)pyrimidine-4-carboxylate (Compound no. 157)

A solution of methyl 2-chloro-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylate (0.1 g, 0.22 mmol) in anhydrous dimethylformamide (1 ml) was treated with morpholine (0.019 g, 0.22 mmol) and triethylamine (0.03 ml, 0.22 mmol) and the mixture was heated at about 60° C. for about 4 hour and cooled to ˜25° C. The reaction was quenched with ice-cooled water and extracted with ethyl acetate. The combined organic layers were washed with water followed by brine, dried over anhydrous sodium sulfate and concentrated to afford the product (68 mg)

EIMS m/z 495.35 (M⁺)
Methyl 2-[(cyclopropylmethyl)amino]-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylate (Compound no. 158)

EIMS m/z 479.35 (M⁺)
Methyl 2-{-4-[(tert-butoxycarbonyl)amino]piperidin-1-yl}-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylate (Compound no. 159)

EIMS m/z 609 (M⁺)

### Step II: Synthesis of 6-[(1R,5S,6s)-6-{[(3,4-dichloro-5-methyl-1H-pyrrol-2-yl)carbonyl]amino}-3-azabicyclo[3.1.0]hex-3-yl]-2-(morpholin-4-yl)pyrimidine-4-carboxylic acid Lithium salt

A solution of methyl 6-[(1R,5S,6s)-6-{[(3,4-dichloro-5-methyl-1H-pyrrol-2-yl)carbonyl]amino}-3-azabicyclo[3.1.0]hex-3-yl]-2-(morpholin-4-yl)pyrimidine-4-carboxylate (0.06 g, 0.12 mmol) in tetrahydrofuran:water (1:1, 2 ml) was treated with lithium hydroxide (0.13 g, 3.02 mmol). The reaction mixture was stirred at about 70° C. for about 24 hours. On completion, the solvent was removed under reduced pressure. The obtained residue was diluted with water, acidified with cold 2N hydrochloric acid and extracted with ethyl acetate. The combined organic layers were washed with water followed by brine, dried over anhydrous sodium sulfate and concentrated. The solid residue thus obtained was triturated with cold ether to afford the title compound (32 mg).

EIMS m/z 481.04 (M⁻)

NMR ¹H (400 MHz, CDCl₃+CD₃OD): δ 1.95-2.01 (m, 2H), 2.24 (s, 3H), 2.55-2.6 (m, 1H), 3.50-3.51 (m, 2H), 3.67-3.90 (m, 8H), 6.57 (s, 1H).

The following compounds were prepared by following a similar synthetic route
2-(4-carboxypiperidin-1-yl)-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylic acid Lithium salt (Compound No. 16)

EIMS m/z 523.15 (M⁻)
6-[(1R,5S,6s)-6-{[(3,4-dichloro-5-methyl-1H-pyrrol-2-yl)carbonyl]amino}-3-azabicyclo[3.1.0]hex-3-yl]-2-(piperidin-1-yl)pyrimidine-4-carboxylic acid Lithium salt (Compound No. 28)

EIMS m/z 479.24 (M⁻)
2-{4-[(tert-butoxycarbonyl)amino]piperidin-1-yl-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylic acid Lithium salt (Compound No. 29)

EIMS m/z 594.04 (M⁻)
6-[(1R,5S,6s)-6-{[(3,4-dichloro-5-methyl-1H-pyrrol-2-yl)carbonyl]amino}-3-azabicyclo[3.1.0]hex-3-yl]-2-(pyrrolidin-1-yl)pyrimidine-4-carboxylic acid Lithium salt (Compound No. 30)

### EIMS m/z 465.20 (M⁻)

### Example 8a

### Synthesis of 6-[(1R,5S,6s)-6-{[(3,4-dichloro-5-methyl-1H-pyrrol-2-yl)carbonyl]amino}-3-azabicyclo[3.1.0]hex-3-yl]-2-(pyrrolidin-1-yl)pyrimidine-4-carboxylic acid Lithium salt (Compound No. 30) and

### 3,4-dichloro-5-methyl-N-{(1R,5S,6s)-3-1⁻²-(pyrrolidin-1-yl)-6-(pyrrolidin-1-ylcarbonyl)pyrimidin-4-yl]-3-azabicyclo[3.1.0]hex-6-yl}-1H-pyrrole-2-carboxamide (Compound no. 31)

Both these compounds were isolated using the synthetic procedure as mentioned for Example 8 using ethyl 6-[(1R,5S,6s)-6-{[(3,4-dichloro-5-methyl-1H-pyrrol-2-yl)carbonyl]amino}-3-azabicyclo[3.1.0]hex-3-yl]-2-(pyrrolidin-1-yl)pyrimidine-4-carboxylate as starting material

### Compound No. 30: MS (M+) 465.20

### Compound no. 31: MS (M+) 518.36, 520.33

### Example 9

### Synthesis of 2-(4-aminopiperidin-1-yl)-6-[(1R,5S,6s)-6-{[(3,4-dichloro-5-methyl-1H-pyrrol-2-yl)carbonyl]amino}-3-azabicylco[3.1.0]hex-3-yl]pyrimidine-4-carboxylic acid trifluoroacetate salt (Compound No. 19)

### Step I: Synthesis of methyl 2-(4-aminopiperidin-1-yl)-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylate

The title compound was prepared by following the similar procedure as described in Step I of Example 8.

### Step II: Synthesis of 2-(4-aminopiperidin-1-yl)-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylic acid trifluoroacetate salt (Compound No. 19)

2-{4-[(tert-butoxycarbonyl)amino]piperidin-1-yl]-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylic acid (0.04 g, 0.06 mmol) was treated with trifluoroacetic acid (5 ml, 20% solution in anhydrous dichloromethane) and stirred at ˜25° C. for about 16 hours. The resultant reaction mixture was concentrated under vacuum to obtain viscous oil, which was triturated with cold ether and filtered to obtain the title compound as a trifluoroacetate salt (20 mg).

EIMS m/z 494.22 (M⁺)

¹H NMR (400 MHz, CDCl₃+CD₃OD): δ 1.55-1.63 (m, 2H), 2.0-2.18 (m, 4H), 2.22 (s, 3H), 2.49-2.51 (m, 1H), 3.05-3.02 (m, 2H), 3.63-3.9 (m, 4H), 4.1-4.22 (m, 1H), 4.59-4.61 (m, 2H), 6.62 (s, 1H).

### Example 10

### Synthesis of 2-[(1R,5S,6s)-6-{[3,4dichloro-5-methyl-1H-pyrrol-2-yl)carbonyl]amino}-3-azabicyclo[3.1.0]hex-3-yl]-4-methyl-1,3-thiazole-5-carboxamide (Compound No. 22)

To a solution of 2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxylic acid (0.075 g, 0.18 mmol) in anhydrous dimethylformamide (5 ml), N-hydroxybenzotriazole (0.049 g, 0.36 mmol), 1-ethyl-3-(3-dimethylaminopropyl) carbodiimide hydrochloride (0.069 g, 0.36 mmol), and N,N-diisopropyl ethyl amine (0.07 g, 0.54 mmol) was added. To this reaction mixture, ammonium carbonate (0.033 g, 0.54 mmol) was added and was stirred at 25° C. for about 20 hours. The reaction was quenched with ice-cooled water and extracted with ethyl acetate. The combined organic layers were washed with 5% aq. sodium bicarbonate solution, water followed by brine, dried over anhydrous sodium sulfate and concentrated to obtain a solid residue. The obtained residue was purified using column chromatography to obtain the title compound (50 mg).

EIMS m/z 414.20 (M⁺)

¹H NMR (400 MHz, CDCl₃+CD₃OD): δ 2.07-2.1 (m, 2H), 2.26 (s, 3H), 2.52 (s, 3H), 2.52-2.60 (m, 1H), 3.53-3.57 (m, 2H), 3.82-3.84 (m, 2H).

### Example 11

### Synthesis of 4-[(3′-chlorobiphenyl-3-yl)methyl]-2-[(1R,5S,6s)-6-{[(3,4-dichloro-5-methyl-1H-pyrrol-2-yl)carbonyl]amino}-1,3-thiazole-5-carboxylic acid Lithium salt (Compound no. 77)

### Step 1: Synthesis of ethyl 2-{(1R,5S,6s)-6-[(tert-butoxycarbonyl)amino]-3-azabicyclo[3.1.0]hex-3-yl}-4-[(3′-chlorobiphenyl-3-yl)methyl]-1,3-thiazole-5-carboxylate

A mixture of ethyl 2-{(1R,5S,6s)-6-[bis(tert-butoxycarbonyl)amino]-3-azabicyclo[3.1.0]hex-3-yl]-4-(3-bromobenzyl)-1,3-thiazole-5-carboxylate (0.2 g, 0.32 mmol), 4-chlorophenyl boronic acid (0.035 g, 0.22 mmol) PdCl₂dppf (0.02 g, 0.03 mmol, 0.1 equiv), potassium carbonate (0.13 g, 0.96 mmol, 3 equiv) in acetonitrile (3 ml) was heated under microwave irradiation at about 120° C. for ˜15 min. Reaction mixture was concentrated and residue was diluted with water and extracted with ethyl acetate. The combined organics were washed with brine, dried and concentrated to give a sticky material that was purified by column chromatography (SiO₂ 100-200 mesh), using hexane-ethylacetate gradient. The product eluted in 15% ethyl acetate/hexane (72 mg).

EIMS m/z 654.44 (M⁺)

### Step II: Synthesis of ethyl 2-[(1R,5S,6s)-6-amino-3-azabicyclo[3.1.0]hex-3-yl]-4-[(3′-chlorobiphenyl-3-yl)methyl]-1,3-thiazole-5-carboxylate

Ethyl 2-{(1R,5S,6s)-6-[(tert-butoxycarbonyl)amino}]-3-azabi cyclo[3.1.0]hex-3-yl]-4-[(3′-chlorobiphenyl-3-yl)methyl]-1,3-thiazole-5-carboxylate (0.32 g, 0.4 mmol) was treated with trifluoroacetic acid (5 ml, 20% solution in anhydrous dichloromethane) and stirred at ˜25° C. for about 16 hours. The resultant reaction mixture was concentrated under vacuum to obtain viscous oil, which was triturated with cold ether and filtered to obtain the title compound as a trifluoroacetate salt (140 mg)

EIMS m/z 454.27 [M+H]⁺

### Step III: Synthesis of ethyl 4-[(3′-chlorobiphenyl-3-yl)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate

To a solution of 3,4-di-chloro-5-methyl-pyrrole-2-carboxylic acid (0.4 g, 0.7 mmol) in anhydrous dimethylformamide (5 ml) 1-ethyl-3-(3-dimethylaminopropyl) carbodiimide hydrochloride (0.16 g, 0.84 mmol) N-hydroxybenzotriazole (0.11 g, 0.84 mmol), N,N-Diisopropyl ethylamine (0.28 g, 2.1 mmol) was added followed by addition of ethyl 2-[(1R,5S,6s)-6-amino-3-azabicyclo[3.1.0]hex-3-yl]-4-[(3′-chlorobiphenyl-3-yl)methyl]-1,3-thiazole-5-carboxylate (0.14 g, 0-0.7 mmol). The reaction mixture was stirred at ˜25° C. for about 16 hours. The reaction was quenched with ice-cooled water and extracted with ethyl acetate. The combined organic layer was washed with water followed by brine, dried over anhydrous sodium sulfate and concentrated to obtain a solid residue that was purified by column choromatography (SiO₂ 100-200 mesh) using 1% methanol/dichloromethane as eluent (150 mg).

EIMS m/z 629.24 [M+H]⁺

Following compounds are parepared by following the above route of synthesis
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-[3-(6-fluoropyridin-3-yl)benzyl]-1,3-thiazole-5-carboxylate (Compound no. 170)

EIMS m/z 614 [M+H]⁺

### Step IV: Synthesis of 4-[(3′-chlorobiphenyl-3-yl)methyl]-2-{[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylic acid Lithium salt

A mixture of ethyl 4-[(3′-chlorobiphenyl-3-yl)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (0.065 g, 0.1 mmol) in tetrahydrofuran:water (1:1, 3 ml) and lithium hydroxide (0.13 g, 3.1 mmol, 30 equiv) was irradiated under microwave conditions at about 110° C. for 15 min. On completion, the solvent was removed under reduced pressure. The obtained residue was diluted with water, acidified with cold 2N HCl, and extracted with ethyl acetate. The combined organic layers were washed water followed by brine, dried over anhydrous sodium sulfate and concentrated. The solid residue thus obtained was triturated with cold ether to afford the title compound (35 mg)

EIMS m/z 601.24 (M⁺)

NMR¹H 400 MHz (CDCl₃CD₃OD) δ 1.9-2.1 (m, 2H), 2.23 (s, 3H), 2.6-2.67 (m, 1H), 3.48-3.7 (m, 2H), 3.75-3.8 (m, 2H), 4.46 (s, 2H), 7.27-7.35 (m, 4H), 7.36-7.4 (m, 2H), 7.50-7.55 (m, 2H).

Following compounds are prepared by following similar route of synthesis

### 2-[(1R,5S,6s)-6-{[(3,4-dichloro-5-methyl-1H-pyrrol-2-yl)carbonyl]amino}-3-azabicyclo[3.1.0]hex-3-yl]-4-[(3′,4′-difluorobiphenyl-3-yl)methyl]-1,3-thiazole-5-carboxylic acid Lithium salt (Compound no. 78)

EIMS m/z 603.24 (M⁺)
4-[(3′-Carboxybiphenyl-3-yl)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 115)

EIMS 611.14 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-[3-(6-fluoropyridin-3-yl)benzyl]-1,3-thiazole-5-carboxylic acid (Compound no. 116)

EIMS 586.69 (M⁻)

### Example 12

### Synthesis of 2-[(1R,5S,6s)-6-{[(3,4-dichloro-5-methyl-1H-pyrrol-2-yl)carbonyl]amino}-3-aza bicyclo[3.1.0]hex-3-yl]-4-[4-(ethylcarbamoyl)benzyl]-1,3-thiazole-5-carboxylic acid Lithium salt (Compound no. 92)

### Step 1: Synthesis of 4-({2-[(1R,5S,6s)-6-{[(3,4-dichloro-5-methyl-1H-pyrrol-2-1)carbon 1]amino}-3-azabicyclo[3.1.0]hex-3-yl]-5-(ethoxycarbonyl)-1,3-thiazol-4-yl}methyl)benzoic acid

A mixture of ethyl 2-[(1R,5S,6s)-6-{[(3,4-dichloro-5-methyl-1H-pyrrol-2-yl)carbonyl]amino}-3-azabicyclo[3.1.0]hex-3-yl]-4-[4-(ethylcarbamoyl)benzyl]-1,3-thiazole-5-carboxylate

(2 g, 3.3 mmol) and sodium hydroxide (0.2 g, 5.07 mmol) in tetrahydrofuran:water:methanol (1:1:1 21 ml) was heated under microwave irradiation at about 100° C. for ˜15 min. The reaction mixture was then concentrated and acidified with ice-cooled 1N HCl solution, filtered to afford the crude product, which was then purified by column chromatography over (SiO₂ 100-200) and using dichloromethane and methanol gradient (1.3 g).

EIMS m/z 562.3 (M⁺)

### Step II: Synthesis of ethyl 2-[(1R,5S,6s)-6-{[(3,4-dichloro-5-methyl-1H-pyrrol-2-yl)carbonyl]amino}-3-azabicyclo[3.1.0]hex-3-yl]-4-[4-(ethylcarbamoyl)benzyl]-1,3-thiazole-5-carboxylate (Compound no. 106)

To a solution of 4-({2-[(1R,5S,6s)-6-{[(3,4-dichloro-5-methyl-1H-pyrrol-2-yl)carbonyl]amino}-3-azabicyclo[3.1.0]hex-3-yl]-5-(ethoxycarbonyl)-1,3-thiazol-4-yl}methyl)benzoic acid (0.06 g, 0.106 mmol), in anhydrous dimethylformamide (2 ml) 1-ethyl-3-(3-dimethylaminopropyl) carbodiimide hydrochloride (0.024 g, 0.127 mmol, 1.2 equiv), N-hydroxybenzotriazole (0.02 g, 0.127 mmol, 1.2 equiv), N,N-Diisopropyl ethylamine (0.04 g, 0.318 mmol, 3 equiv) was added followed by addition of ethyl amine hydrochloride (0.008 g, 0.106 mmol, 1 equiv). The reaction mixture was stirred at ˜25° C. for about 16 hours. The reaction was quenched with ice-cooled water and extracted with ethyl acetate. The combined organic layer was washed with water followed by brine, dried over anhydrous sodium sulfate and concentrated to obtain a solid residue that was triturated with cold hexane/ether mixture (1:1) to afford the product (30 mg).

EIMS m/z 590.25, (M⁺)

### Step Synthesis of 2-[(1R,5S,6s)-6-{[(3,4-dichloro-5-methyl-1H-pyrrol-2-yl)carbonyl]amino}-3-azabicyclo[3.1.0]hex-3-yl]-4-[4-(ethylcarbamoyl)benzyl]-1,3-thiazole-5-carboxylic acid

A mixture of ethyl 2-[(1R,5S,6s)-6-{[(3,4-dichloro-5-methyl-1H-pyrrol-2-yl)carbonyl]amino}-3-azabicyclo[3.1.0]hex-3-yl]-4-[4-(ethylcarbamoyl)benzyl]-1,3-thiazole-5-carboxylate (0.03 g, 0.051 mmol) and sodium hydroxide (0.004 g. 0.1 mmol) in tetrahydrofuran:water (2:1, 3 ml) was heated at about 70° C. for about 16 hours and then cooled to room temperature. The reaction mixture was concentrated under vacuum, and aqueous part was acidified with 1N HCl solution under ice-cooled conditions and filtered under vacuum. The residue was tritureated with cold diethyl ether to afford the product (15 mg).

### Step IV: Synthesis of 2-[(1R,5S,6s)-6-{[(3,4-dichloro-5-methyl-1H-pyrrol-2-yl)carbonyl]amino}-3-azabicyclo[3.1.0]hex-3-yl]-4-[4-(ethylcarbamoyl)benzyl]-1,3-thiazole-5-carboxylic acid Lithium salt

A solution of 2-[(1R,5S,6s)-6-{[(3,4-dichloro-5-methyl-1H-pyrrol-2-yl)carbonyl]amino}-3-azabicyclo[3.1.0]hex-3-yl]-4-[4-(ethylcarbamoyl)benzyl]-1,3-thiazole-5-carboxylic acid (0.015 g, 0.026 mmol) in tetrahydrofuran (2 ml) was treated with aqueous lithium hydroxide (0.001 g, 0.026 mmol) and stirred at room temperature (˜25° C.) for about 60 min. The solvent was removed and the product was dried under vacuum to afford the salt (13 mg).

EIMS m/z 562.52 (M⁺)

NMR ¹H 400 MHz (CDCl₃+CD₃OD) δ 1.21-1.26 (t, 3H, J=7.2 Hz), 2.1-2.15 (m, 2H), 2.25 (s, 3H), 2.57-2.63 (m, 1H), 3.41-3.45 (m, 2H), 3.80-3.90 (m, 4H), 4.46 (s, 2H).

Following compounds can be prepared by following similar route of synthesis
4-(4-carbamoylbenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 93)

EIMS m/z 534.33 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-[4-(methylcarbamoyl)benzyl]-1,3-thiazole-5-carboxylic acid (Compound no. 94)

EIMS m/z 548.44 (M⁺)
2-[(1R,5S,6s)-6-{[(3,4-dichloro-5-methyl-1H-pyrrol-2-yl)carbonyl]amino}-3-azabicyclo[3.1.0]hex-3-yl]-4-{4-[(4-hydroxypiperidin-1-yl)carbonyl]benzyl]-1,3-thiazole-5-carboxylic acid (Compound no. 117)

EIMS m/z 617.97 (M⁻)

### Example 13

### Synthesis of 2-[(1R,5S,6s)-6-{[(3,4-dichloro-5-methyl-1H-pyrrol-2-yl)carbonyl]amino}-3-azabicyclo[3.1.0]-hex-3-yl]-4-[2-(ethylamino)-2-oxoethyl]-1,3-thiazole-5-carboxylic acid (Compound no. 113)

### Step I: Synthesis of {2-[(1R,5S,6s)-6-{[(3,4-dichloro-5-methyl-1H-pyrrol-2-yl)carbonyl]amino}-3-azabicyclo[3.1.0]hex-3-yl]-5-(methoxycarbonyl)-1,3-thiazol-4-acetic acid

A mixture of methyl 2-[(1R,5S,6s)-6-{[(3,4-dichloro-5-methyl-1H-pyrrol-2-yl)carbonyl]amino}-3-azabicyclo[3.1.0]hex-3-yl]-4-(2-methoxy-2-oxoethyl)-1,3-thiazole-5-carboxylate (0.2 g, 0.42 mmol) and lithium hydroxide (0.019 g, 0.46 mmol) in tetrahydrofuran:methanol:water (1:1:1, 6 ml) was stirred at room temperature (˜25° C.) for about 16 hours. On completion, the solvent was removed under reduced pressure. The obtained residue was diluted with water, acidified with cold 2N HCl, and extracted with ethyl acetate. The combined organic layers were washed water followed by brine, dried over anhydrous sodium sulfate and concentrated. The solid residue thus obtained was triturated with cold ether to afford the acid (150 mg).

EIMS m/z 473.15 (M⁻)

### Step II: Synthesis of Methyl 2-[(1R,5S,6s)-6-1 [(3,4-dichloro-5-methyl-1H-pyrrol-2-yl)carbonyl]amino}-3-azabicyclo[3.1.0]hex-3-yl]4-[2-(ethylamino)-2-oxoethyl]-1,3-thiazole-5-carboxylate

To a solution of {2-[(1R,5S,6s)-6-{[(3,4-dichloro-5-methyl-1H-pyrrol-2-yl)carbonyl]amino}-3-azabicyclo[3.1.0]hex-3-yl]-5-(methoxycarbonyl)-1,3-thiazol-4-yl}acetic acid (0.05 g, 0.105 mmol) in anhydrous dimethylformamide (2 ml), N-hydroxybenzotriazole (0.017 g, 0.12 mmol), 1-ethyl-3-(3-dimethylamino-propyl) carbodiimide hydrochloride (0.024 g, 0.12 mmol) and N,N-diisopropyl ethylamine (0.04 g, 0.31 mmol) was added. To this reaction mixture, ethyl amine (0.05 ml, 0.12 mmol, 2M) solution in tetrahydrofuran was added and the reaction mixture was stirred at ˜25° C. for about 16 hours. The reaction was quenched with ice-cooled water and extracted with ethyl acetate. The combined organic layers were washed with 5% aq. sodium bicarbonate solution; water followed by brine, dried over anhydrous sodium sulfate and concentrated to obtain a solid residue that after triturating with ether afforded the title compound (30 mg).

EIMS m/z 501.96 (M⁻)

### Step III: Synthesis of 2-[(1R,5S,6s)-6-{[(3,4-dichloro-5-methyl-1H-pyrrol-2-yl)carbonyl]amino}-3-azabicyclo[3.1.0]hex-3-yl]4-[2-(ethylamino)-2-oxoethyl]-1,3-thiazole-5-carboxylic acid

A mixture of methyl 2-[(1R,5S,6s)-6-{[(3,4-dichloro-5-methyl-1H-pyrrol-2-yl)carbonyl]amino}-3-azabicyclo[3.1.0]hex-3-yl]4-[2-(ethylamino)-2-oxoethyl]-1,3-thiazole-5-carboxylate (0.02 g, 0.04 mmol) and lithium hydroxide (0.008 g, 0.2 mmol) in tetrahydrofuran:methanol:water (1:1:1, 3 ml) was stirred at about 70° C. for about 16 hours. On completion, the solvent was removed under reduced pressure. The obtained residue was diluted with water, acidified with cold 2N HCl, and extracted with ethyl acetate. The combined organic layers were washed water followed by brine, dried over anhydrous sodium sulfate and concentrated. The solid residue thus obtained was triturated with cold ether to afford the acid. (13 mg).

EIMS m/z 486.09 (M⁻)

NMR ¹H (400 MHz, CD₃OD) δ 1.0-1.18 (t, 3H, 7.4 Hz), 1.98-2.01 (m, 2H), 2.12 (s, 3H), 2.45-2.52 (m, 1H), 3.59-3.75 (m, 2H), 3.75-3.84 (m, 2H), 3.86-(s, 2H).

Following compounds can be prepared by following the same route of synthesis as above
4-(2-{4-[(tert-butoxycarbonyl)amino]piperidin-1-yl]-2-oxoethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 114)

EIMS m/z 641.17 (M⁻)
4-[2-(cyclopropylamino)-2-oxoethyl]-2-[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 119)

EIMS m/z 498.56 (M⁻)
4-[2-(cyclopentylamino)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 120)

EIMS m/z 528.13 (M⁻)
4-{2-[(carboxymethyl)amino]-2-oxoethyl}-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 121)
N-({2-[(1R,5S,6s)-6-{[(3,4-dichloro-5-methyl-1H-pyrrol-2-yl)carbonyl]amino}-3-azabicyclo[3.1.0]hex-3-yl]-5-(methoxycarbonyl)-1,3-thiazol-4-yl]acetyl)glycine Lithium salt (Compound no. 122)

ELMS m/z 531.03 (M⁻)
4-[2-(cyclobutylamino)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 123)
2-[(1R,5S,6s)-6-{[(3,4-dichloro-5-methyl-1H-pyrrol-2-yl)carbonyl]amino}-3-azabicyclo[3.1.0]hex-3-yl]-4-(2-oxo-2-{[3-(trifluoromethyl)phenyl]amino}ethyl)-1,3-thiazole-5-carboxylic acid Lithium salt (Compound no. 124)

EIMS m/z 512.29 (M⁻)
4-{2[(3-chlorophenyl)amino]-2-oxoethyl}-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 125)
2-[(1R,5S,6s)-6-{[(3,4-dichloro-5-methyl-1H-pyrrol-2-yl)carbonyl]amino}-3-azabicyclo[3.1.0]hex-3-yl]4-[2-(4-hydroxypiperidin-1-yl)-2-oxoethyl]-1,3-thiazole-5-carboxylic acid (Compound no. 126)

EIMS m/z 542.05 (M⁻)
2-[(1R,5S,6s)-6-{[(3,4-dichloro-5-methyl-1H-pyrrol-2-yl)carbonyl]amino}-3-azabicyclo[3.1.0]hex-3-yl]4-[2-(morpholin-4-yl)-2-oxoethyl]-1,3-thiazole-5-carboxylic acid (Compound no. 127)

EIMS m/z 528.43 (M⁻)
2-[(1R,5S,6s)-6-{[(3,4-dichloro-5-methyl-1H-pyrrol-2-yl)carbonyl]amino}-3-azabicyclo[3.1.0]hex-3-yl]4-[2-(methoxyamino)-2-oxoethyl]-1,3-thiazole-5-carboxylic acid (Compound no. 128)

EIMS m/z 488 (M⁻)
2-[(1R,5S,6s)-6-{[(3,4-dichloro-5-methyl-1H-pyrrol-2-yl)carbonyl]amino}-3-azabicyclo[3.1.0]hex-3-yl]-4-[2-oxo-2-(pyridin-3-ylamino)ethyl]-1,3-thiazole-5-carboxylic acid (Compound no. 129)

EIMS m/z 535.22 (M⁻)
4-[2-(azetidin-1-yl)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 130)

EIMS m/z 498.28 (M⁻)
2-[(1R,5S,6s)-6-{[(3,4-dichloro-5-methyl-1H-pyrrol-2-yl)carbonyl]amino}-3-azabicyclo[3.1.0]hex-3-yl]4-[2-(4-methylpiperazin-1-yl)-2-oxoethyl]-1,3-thiazole-5-carboxylic acid (Compound no. 131)

EIMS m/z 541.35 (M⁻)
4-{2-[4-(tert-butoxycarbonyl)piperazin-1-yl]-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 132)

EIMS m/z 627.44 (M⁻)
2-[(1R,5S,6 s)-6-{[(3,4-dichloro-5-methyl-1H-pyrrol-2-yl)carbonyl]amino}-3-azabicyclo[3.1.0]hex-3-yl]-4-(2-oxo-2-{[4-(4H-1,2,4-triazol-4-yl)phenyl]amino}ethyl)-1,3-thiazole-5-carboxylic acid (Compound no. 133)
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(4-methoxybenzyl)amino]-2-oxoethyl}-1,3-thiazole-5-carboxylic acid (Compound no. 134)
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(4-methyl-1,3-thiazol-2-yl)amino]-2-oxoethyl}-1,3-thiazole-5-carboxylic acid (Compound no. 135)
2-[(1R,5S,6s)-6-{[(3,4-dichloro-5-methyl-1H-pyrrol-2-yl)carbonyl]amino}-3-azabicyclo[3.1.0]hex-3-yl]-4-(2-oxo-2-{[4-(trifluoromethyl)benzyl]amino}ethyl)-1,3-thiazole-5-carboxylic acid (Compound no. 136)
2-[(1R,5S,6s)-6-[(3,4-dichloro-5-methyl-1H-pyrrol-2-yl)carbonyl]amino}-3-azabicyclo[3.1.0]hex-3-yl]-4-{2-[(3-fluorophenyl)amino]-2-oxoethyl}-1,3-thiazole-5-carboxylic acid (Compound no. 137)
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(4-ethylphenyl)amino]-2-oxoethyl}-1,3-thiazole-5-carboxylic acid (Compound no. 138)
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(3-methylbenzyl)amino]-2-oxoethyl}-1,3-thiazole-5-carboxylic acid (Compound no. 139)
2-[(1R,5S,6s)-6-{[(3,4-dichloro-5-methyl-1H-pyrrol-2-yl)carbonyl]amino}-3-azabicyclo[3.1.0]hex-3-yl]4-[2-oxo-2-(piperidin-1-yl)ethyl]-1,3-thiazole-5-carboxylic acid (Compound no. 140)
4-(2-{3-[(tert-butoxycarbonyl)amino]pyrrolidin-1-yl]-2-oxoethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 141)
4-[2-(3-aminopyrrolidin-1-yl)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 142)
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(1-ethyl-1H-pyrazol-5-yl)amino]-2-oxoethyl}-1,3-thiazole-5-carboxylic acid (Compound no. 143)
4-[2-(4-aminopiperidin-1-yl)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 144)
2-[(1R,5S,6s)-6-{[(3,4-dichloro-5-methyl-1H-pyrrol-2-yl)carbonyl]amino}-3-azabicyclo[3.1.0]hex-3-yl]-4-[2-oxo-2-(piperazin-1-yl)ethyl]-1,3-thiazole-5-carboxylic acid (Compound no. 145)
2-[(1R,5S,6s)-6-{[(3,4-dichloro-5-methyl-1H-pyrrol-2-yl)carbonyl]amino}-3-azabicyclo[3.1.0]hex-3-yl]4-[2-oxo-2-(1,3-thiazol-2-yl)ethyl]-1,3-thiazole-5-carboxylic acid (Compound no. 146)
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(3-methyl-1,2-oxazol-5-yl)amino]-2-oxoethyl}-1,3-thiazole-5-carboxylic acid (Compound no. 147)
4-[2-(1H-benzimidazol-2-ylamino)-2-oxoethyl]-2-{[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 148)
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(2-hydroxyethyl)amino]-2-oxoethyl}-1,3-thiazole-5-carboxylic acid (Compound no. 149)
4-{2-[(3-chlorobenzyl)amino]-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 150)
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(1-hydroxy-2-methylpropan-2-yl)amino]-2-oxoethyl}-1,3-thiazole-5-carboxylic acid (Compound no. 151)
4-{2-[(4-Carboxyphenyl)amino]-2-oxoethyl}-2-[(1R,5S,6s)-6-{(3,4-dichloro-5-methyl-1H-pyrrol-2-yl)carbonyl]amino}-3-azabicyclo[3.1.0]hex-3-yl]-1,3-thiazole-5-carboxylic acid (Compound no. 152)

### Example 14

### Synthesis of N-[3-({2-[(1R,5S,6s)-6-{[(3,4-dichloro-5-methyl-1H-pyrrol-2-yl)carbonyl]amino}-3-azabicyclo[3.1.0]hex-3-yl]-5-(ethoxycarbonyl)-1,3-thiazol-4-yl]methyl)phenyl]glycine (Compound no. 103)

### Step 1: Synthesis of ethyl 4-(3-aminobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate

A mixture of ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(3-nitrobenzyl)-1,3-thiazole-5-carboxylate (0.2 g, 0.35 mmol), stannous chloride (0.64 g, 2.8 mmol), HCl (5 ml) was heated at about 70° C. for about 3 hours. After completion of the reaction, the reaction mixture was treated with 6N sodium hydroxide solution under ice-cooled conditions to pH ˜10, partitioned with dichloromethane. The combined organics were dried over anhydrous sodium sulphate, and concentrated to afford the title compound (92 mg).

EIMS m/z 534.14 (M⁻)

### Step II: Synthesis of ethyl 2-[(1R,5S,6s)-6-{[(3,4-dichloro-5-methyl-1H-pyrrol-2-yl)carbonyl]amino}-3-azabicyclo[3.1.0]hex-3-yl]-4-{3-[(2-methoxy-2-oxoethyl)amino]benzyl}-1,3-thiazole-5-carboxylate

To a solution of ethyl 4-(3-aminobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (0.1 g, 0.18 mmol) in anhydrous dimethylformamide (5 ml) methyl bromo acetate (0.028 g, 0.187 mmol) was added followed by addition of DIEA (0.023 g, 0.187 mmol). The reaction mixture was heated at about 40° C. for about 16 hours, quenched with ice-cooled water and extracted with ethyl acetate. The combined organic layers were washed with water followed by brine, dried over anhydrous sodium carbonate and concentrated. to get the title compound (35 mg).

EIMS m/z 608 (M⁻)

### Step III: Synthesis of N-[3-({2-[(1R,5S,6s)-6-{[(3,4-dichloro-5-methyl-1H-pyrrol-2-yl)carbon 1 amino}-3-azabicyclo[3.1.0]hex-3-yl]-5-1)-1,3-thiazol-4-yl]methyl)phenyl]glycine

A mixture of ethyl 2-[(1R,5S,6s)-6-{[(3,4-dichloro-5-methyl-1H-pyrrol-2-yl)carbonyl]amino}-3-azabicyclo[3.1.0]hex-3-yl]-4-{3-[(2-methoxy-2-oxoethyl)amino]benzyl}-1,3-thiazole-5-carboxylate

(0.02 g, 0.03 mmol) and lithium hydroxide (0.002 g, 0.06 mmol) in tetrahydrofuran:water (1:1, 3 ml) was stirred at room temperature (˜25° C.) for about 20 hours. On completion, the solvent was removed under reduced pressure. The obtained residue was diluted with water, acidified with cold 2N HCl, and extracted with ethyl acetate. The combined organic layers were washed water followed by brine, dried over anhydrous sodium sulfate and concentrated. The solid residue thus obtained was triturated with cold ether to afford the acid (13 mg).

EIMS m/z 534.33 (M⁻)

¹H NMR (400 MHz, CDCl₃) δ 1.2-1.4 (t, 3H), 2.01-2.15 (m, 2H), 2.26 (s, 3H), 2.5-2.6 (m, 1H), 3.57-3.76 (m, 4H), 4.03 (s, 2H), 4.27-4.39 (m, 4H), 7.01-7.3 (m, 1H), 7.12-7.15 (m, 1H), 7.31-7.4 (m, 1H), 7.77-7.79 (m, 1H).

### Example 15

### Synthesis of 4-[3-(acetylamino)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 107)

### Step I: Synthesis of ethyl 4-(3-aminobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate Procedure same as Step I, Example 14

### Step II: Synthesis of ethyl 4-[3-(acetylamino)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate

An ice-cooled solution of ethyl 4-(3-aminobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (0.1 g, 0.18 mmol) in anhydrous tetrahydrofuran (3 ml) and triethyl amine (0.018 g, 0.18 mmol) was treated dropwise with acetyl chloride (0.017 g, 0.22 mmol) and stirred at room temperature (˜25° C.) for about 16 hours, concentrated under vacuum. The residue was partitioned between water and ethyl acetate. The combined organics were washed with water, brine, dried and concentrated to afford the product (30 mg).

EIMS m/z 578.04 (M⁻)

### Step III: Synthesis of 4-[3-(acetylamino)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid

A mixture of ethyl 4-[3-(acetylamino)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (0.03 g, 0.05 mmol) and lithium hydroxide (0.021 g, 0.52 mmol) in tetrahydrofuran:water (1:1, 3 ml) was stirred at about 70° C. for about 20 hours. On completion, the solvent was removed under reduced pressure. The obtained residue was diluted with water, acidified with cold 2N HCl, and extracted with ethyl acetate. The combined organic layers were washed water followed by brine, dried over anhydrous sodium sulfate and concentrated. The solid residue thus obtained was triturated with cold ether to afford the acid (13 mg).

EIMS m/z 548.39 (M⁻)

¹H NMR (400 MHz, CDCl₃+CD₃OD) δ 1.12-1.3 (t, 3H, 7.4 Hz), 1.94-1.99 (m, 2H), 2.26 (s, 3H), 2.58-2.63 (m, 1H), 2.95-2.99 (q, 2H, 7.4 Hz), 3.40-3.42 (m, 2H), 3.51-3.59 (m, 2H), 4.31 (s, 2H), 6.97-7.05 (m, 1H), 7.14-7.16 (m, 2H), 7.40-7.41 (m, 2H).

### Example 16

### Synthesis of 2-[(1R,5S,6s)-6-{[(3,4-dichlor-5-methyl-1H-pyrrol-2-yl)carbonyl]amino}-3-azabicyclo[3.1.0]hex-3-yl]-4-{3-[(ethylcarbamoyl)amino]benzyl}-1,3-thiazole-5-carboxylic acid (Compound no. 110)

### Step I: Synthesis of ethyl 4-(3-aminobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate

Procedure same as Step I, Example 14

### Step II: Synthesis of ethyl 2-[(1R,5S,6s)-6-{[(3,4-dichloro-5-methyl-1H-pyrrol-2-yl)carbonyl]amino}-3-azabicyclo[3.1.0]hex-3-yl]-4-{3-[(ethylcarbamoyl)amino]benzyl}-1,3-thiazole-5-carboxylate

To a solution of ethyl 4-(3-aminobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (0.06 g, 0.11 mmol) in tetrahydrofuran (5 ml), p-nitrophenyl chloroformate (0.027 g, 0.13 mmol) was added followed by dropwise addition of pyridine (0.017 g, 0.22 mmol) and stirred at room temperature (˜25° C.) for about 2 hours. The solvent was removed under vacuum and the residue was taken up in ethanol (5 ml). This was treated with ethyl amine (0.22 ml, 2M solution in tetrahydrofuran) and the reaction mixture was refluxed for about 16 hours. Upon completion of the reaction, ethanol was removed under vacuum, residue partitioned between ethyl acetate and water. The combined organics were washed with water, brine, dried and concentrated under vasccum, triturated with diethyl ether to afford the product (35 mg).

EIMS m/z 605, (M⁻)

### Step III: Synthesis of 2-[(1R,5S,6s)-6-{[(3,4-dichloro-5-methyl-1H-pyrrol-2-yl)carbonyl]amino}-3-azabicyclo[3.1.0]hex-3-yl]-4-{3-[(ethylcarbamoyl)amino]benzyl}-1,3-thiazole-5-carboxylic acid

A mixture of ethyl 2-[(1R,5S,6s)-6-{[(3,4-dichloro-5-methyl-1H-pyrrol-2-yl)carbonyl]amino}-3-azabicyclo[3.1.0]hex-3-yl]-4-{3-[(ethylcarbamoyl)amino]benzyl}-1,3-thiazole-5-carboxylate

(0.035 g, 0.057 mmol) and lithium hydroxide (0.024 g, 0.57 mmol) in tetrahydrofuran:water (1:1, 3 ml) was stirred at about 70° C. for about 20 hours. On completion, the solvent was removed under reduced pressure. The obtained residue was diluted with water, acidified with cold 2N HCl, and extracted with ethyl acetate. The combined organic layers were washed water followed by brine, dried over anhydrous sodium sulfate and concentrated. The solid residue thus obtained was triturated with cold ether to afford the acid (12 mg).

EIMS m/z 577.25 (m⁻)

¹H NMR (400 MHz, CDCl₃+CD₃OD) δ 1.22-1.26 (m, 2H), 2.21-2.24 (s, 3H), 2.59 (bs, 1H), 3.71-3.85 (m, 4H), 4.19-4.39 (m, 2H), 7.10-7.12 (m, 1H), 7.14-7.20 (m, 1H), 7.24-7.33 (m, 1H), 7.62-7.64 (m, 1H).

Following compound is prepared by following the similar route of synthesis as above
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-{4-[(ethylcarbamoyl)amino]benzyl}-1,3-thiazole-5-carboxylate (Compound no. 118)

EIMS m/z 605.21, 607.11 (M⁻)

### Assay Methods

DNA Supercoiling Inhibition Assay (Gyrase inhibition assay):

Gyrase supercoiling assays was performed as described by Inspiralis, Norwich, UK (Inspiralis Product No. #G1001). Samples (30 μl) containing 1 unit of DNA gyrase and 0.5 μg of relaxed pBR322DNA in assay buffer (35 mM Tris-HCl, pH 7.5, 24 mM KCl, 4 mM MgCl₂, 2 mM DTT, 1.8 mM spermidine, 1 mM ATP, 6.5% glycerol and 0.1 mg/ml albumin) was incubated at 37° C. for 30 min with and without inhibitors. Samples was loaded onto 0.8% agarose gels and run in the absence of ethidium bromide. The gels was stained in ethidium bromide and visualized in Bio-rad gel doc system. The conversion or inhibition of supercoiling DNA was estimated from the bands visible and the IC₅₀ was calculated using Bio-Rad's Quantity one software.

The compounds provided herein showed activity (IC₅₀) between 0.06 μM->15 μM.

DNA Relaxation Inhibition Assay (TopoIV inhibition assay):

DNA relaxation assays was performed as described by Inspiralis, Norwich, UK (Inspiralis Product No. #D4001). Samples (30 μl) containing 1 unit of Topoisomerase IV and 0.4 μg of supercoiled pBR322DNA in assay buffer (40 mM HEPES-KOH, pH 7.6, 100 mM Potassium Glutamate, 10 mM Mg acetate, 10 mM DTT, 2 mM ATP and 50 μg/ml albumin) was incubated at 37° C. for 30 min with and without inhibitors. Samples were loaded onto 0.8% agarose gels and run in the absence of ethidium bromide. The gels was stained in ethidium bromide and visualized in Bio-rad gel doc system. The conversion or inhibition of supercoiling DNA was estimated from the bands visible and the IC₅₀ was calculated using Bio-Rad's Quantity one software

The compounds provided herein showed activity (IC₅₀) between 0.125 μM->15 μM.

In vitro Antibacterial Activity (MIC method)
Microbroth dilution method

### Medium:

1. Cation adjusted Mueller Hinton Broth (MHB-Difco): staphylococci spp., enterococci spp.
2. Cation adjusted Mueller Hinton Broth +5% lysed horse blood for fastidious pathogens: streptococci spp.

### Preparation of Compounds:

1 mg/mL of stock solution of compounds and standard drug were prepared in dimethylsulfoxide/distilled water/solvent and further 2 fold dilutions were done in respective broth in 96 well microtiter plates as per CLSI guidelines. The stock solution was changed according to the need of the experiment.

### Inoculum Preparation:

Saline suspensions was prepared from three to four isolated colonies taken from 18-24 hrs agar plates. The turbidity of the suspension was adjusted to 0.5-1.0 Mc Farland standard (1.5×10⁸ CFU/mL). Cultures was diluted 100 times (respective medium) and 100 μl of diluted culture broth was added in wells already containing 100 μl of broth (positive control) or broth containing compound to get approximately 3−7×10⁵ CFU/ml. Cultures was randomly selected for CFU determination of inoculum suspensions. Micro titer plates was then incubated at 35-37° C. for 16-20 hrs in ambient air.

End Point determination of MIC:

Concentration of the drug at which there was complete disappearance of growth was considered as MIC.

### REFERENCES

1. Clinical and Laboratory Standards Institute, Methods for Dilution Antimicrobial Susceptibility Tests for Bacteria That Grow Aerobically; Approved Standard-Seventh Edition. M7-A7, Vol. 26. No. 2 (January 2006)
2. Clinical and Laboratory Standards Institute, Performance Standards for Antimicrobial Susceptibility Testing—Sixteenth informational supplement, M100-S17, Vol. 27 No. 1, January 2007

### Results:

a) The compounds described herein exhibited MIC values against Staphylococcus aureus (ATCC29213), S. aureus (MRSA 562), in the range of between about 0.25 μg/mL to about 16 μg/mL.
b) The compounds described herein exhibited MIC values against S. aureus (ATCC MRSA 43300)), in the range of between about 0.5 μg/mL to about 16 μg/mL.
c) The compounds described herein exhibited MIC values against S. epidermidis (ATCC 12228) in the range of between about 0.125 μg/mL to about 16 μg/mL.
d) The compounds described herein exhibited MIC values against S. epidermidis (ATCC MRSE 35984) in the range of between about 1 μg/mL to about 16 μg/mL.
e) The compounds described herein exhibited MIC values against Streptococcus pyogenes (ATCC 19615) in the range of between about 2 μg/mL to about 32 μg/mL.
f) The compounds described herein exhibited MIC values against Streptococcus pyogenes (2534) in the range of between about 4 μg/mL to about 32 μg/mL.
g) The compounds described herein exhibited MIC values against S. viridans (659) in the range of between about 16 μg/mL.
h) The compounds described herein exhibited MIC values against Streptococcus pneumoniae (ATCC 49619) in the range of between about 2 μg/mL to about 32 μg/mL.
i) The compounds described herein exhibited MIC values against E. faecalis (ATCC 29212) in the range of between about 0.25 μg/mL to about 16 μg/mL.
j) The compounds described herein exhibited MIC values against E. faecium (6A VRE) in the range of between about 2 μg/mL to about 16 μg/mL.

We claim:

1. A compound of Formula 1 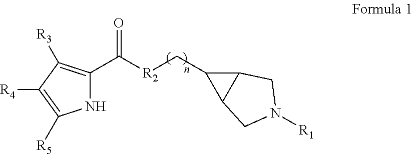
and its pharmaceutically acceptable salts, pharmaceutically acceptable solvates, co-crystals, enantiomers, diastereomers, polymorphs, prodrugs, metabolites or N-oxides
wherein,
n is 0-2;
R₁ is cycloalkyl, aryl, heteroaryl or heterocyclyl which may optionally be substituted with 1-3 substituent independently selected from alkyl, —(CH₂)ₓ—C(═O)OR_{d}, —(CH₂)ₓ—CONR_{f}R_{q}, cyano, halo, CH₂—OH, —CO-cycloalkyl, —CO-heteroaryl, —(CH₂)ₓ—CO-heterocyclyl, —CF₃, —OCF₃ or R₈—(R₉)ₘ;
wherein
R₈ is heteroaryl, heterocyclyl, aryl, alkyl, cycloalkyl, (C₁₋₆)alkyl-cycloalkyl, (C₁₋₆)alkyl-heteroaryl, (C₁₋₆)alkyl-heterocyclyl or (C₁₋₆)alkyl-aryl;
R₉ is hydrogen, alkyl, hydroxy, —C(═O)OR_{d}, halo, —NR_{f}R_{q}, —CO-heteroaryl, alkoxy, —NHCORₐ, aryl, heteroaryl, heterocyclyl, cycloalkyl, aryloxy, heteroaryloxy, heterocyclyloxy, aralkyloxy, heteroarylalkyloxy, heterocyclylalkyloxy, —CF₃, —OCF₃, alkylcarbonyl, —CONR_{f}R_{q}, —NHCONR_{f}R_{q}, —NH(CH₂)ₓC(═O)OR_{d}, —NH(CH₂)ₓcycloalkyl, —NH(CH₂)ₓheteroaryl, —NH(CH₂)ₓheterocyclyl or —NH(CH₂)ₓaryl;
Rₐ is alkyl, alkoxy or NR_{f}R_{q};
R_{d} is hydrogen or alkyl;
R_{f} and R_{q} are independently hydrogen, alkyl, alkoxy, cycloalkyl, aryl, heteroaryl and heterocyclyl, (C₁₋₆)alkyl-cycloalkyl, (C₁₋₆)alkyl-heteroaryl, (C₁₋₆)alkyl-heterocyclyl, (C₁₋₆)alkyl-aryl, (CH₂)ₓ—OH, (CH₂)ₓ—C(═O)OR_{d}, heteroarylalkyl, heteroaryl-heteroaryl, heterocyclylalkyl, arylalkyl, cycloalkylalkyl or R_{f} and R_{q} together with ‘N’ to which they are attached form a ring which optionally contain heteroatom selected from N, O and S;
x can be 0-2;
m can be 1-4;
R₂ is O, NH or CHR″ (wherein R″ can be hydrogen, alkyl, cyano, nitro, amino, hydroxyl or alkoxy);
R₃, R₄, R₅ are independently hydrogen, alkyl, alkenyl, alkynyl, halogen, cyano, amino, hydroxy, alkoxy, carbonyl, thiocarbonyl, oxo, cycloalkyl, aryl, heteroaryl, heterocyclyl, arylalkyl, heteroarylalkyl, cycloalkylalkyl or heterocyclylalkyl or R₃ and R₄ or R₄ and R₅ taken together with the carbon atoms to which they are attached can form an aromatic or non-aromatic ring, which optionally contain heteroatom selected from N, O and S.

2. A compound selected from the group consisting of:
2-[(1R,5S,6s)-6-{[(3,4-dichloro-5-methyl-1H-pyrrol-2-yl)carbonyl]amino})-3-azabicyclo[3.1.0]hex-3-yl]-4-methyl-1,3-thiazole-5-carboxylic acid Lithium salt (Compound No. 1),
2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 2),
2-[(1R,5S,6s)-6-{[(4-chloro-5-methyl-1H-pyrrol-2-yl)carbonyl]amino}-3-azabicyclo[3.1.0]hex-3-yl]pyridine-3-carboxamide (Compound No. 3),
2-[(1R,5S,6s)-6-{[(3,4-dichloro-5-methyl-1H-pyrrol-2-yl)carbonyl]amino})-3-azabicyclo[3.1.0]hex-3-yl]pyridine-3-carboxamide (Compound No. 4),
3,4-Dichloro-N-[(1R,5S,6s)-3-(5-cyanopyridin-2-yl)-3-azabicyclo[3.1.0]hex-6-yl]-5-methyl-1H-pyrrole-2-carboxamide (Compound No. 5),
2-[(1R,5S,6s)-6-[(4-chloro-5-methyl-1H-pyrrol-2-yl)carbonyl]amino-3-azabicyclo [3.1.0]hex-3-yl]-4-methyl-1,3-thiazole-5-carboxylic acid Lithium salt (Compound No. 6),
2-Chloro-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylic acid Lithium salt (Compound No. 7),
2-Chloro-6-[(1R,5S,6s)-6-[(3,4-dichloro-5-methyl-1H-pyrrol-2-yl)carbonyl]amino}-3-azabicyclo[3.1.0]hex-3-yl]pyridine-4-carboxylic acid Lithium salt (Compound No. 8),
6-[(1R,5S,6s)-6-{[(3,4-dichloro-5-methyl-1H-pyrrol-2-yl)carbonyl]amino}-3-azabicyclo[3.1.0]hex-3-yl]pyridine-2-carboxylic acid Lithium salt (Compound No. 9),
6-[(1R,5S,6s)-6-{[(3,4-dichloro-5-methyl-1H-pyrrol-2-yl)carbonyl]amino}-3-azabicyclo[3.1.0]hex-3-yl]pyridine-3-carboxylic acid Lithium salt (Compound No. 10),
6-[(1R,5S,6s)-6-{[(3,4-dichloro-5-methyl-1H-pyrrol-2-yl)carbonyl]amino}-3-azabicyclo[3.1.0]hex-3-yl]-2-morpholin-4-ylpyrimidine-4-carboxylic acid Lithium salt (Compound No. 11),
{2-[(1R,5S,6s)-6-{[(3,4-dichloro-5-methyl-1H-pyrrol-2-yl)carbonyl]amino}-3-azabicyclo[3.1.0]hex-3-yl]-1,3-thiazol-4-yl]acetic acid Lithium salt (Compound No. 12),
5-[(1R,5S,6s)-6-{[(3,4-dichloro-5-methyl-1H-pyrrol-2-yl)carbonyl]amino}-3-azabicyclo[3.1.0]hex-3-yl]furan-2-carboxylic acid Lithium salt (Compound No. 13),
5-[(1R,5S,6s)-6-{[(3,4-dichloro-5-methyl-1H-pyrrol-2-yl)carbonyl]amino}-3-azabicyclo[3.1.0]hex-3-yl]thiophene-2-carboxylic acid Lithium salt (Compound No. 14),
6-[(1R,5S,6s)-6-{[(3,4-dichloro-5-methyl-1H-pyrrol-2-yl)carbonyl]amino}-3-azabicyclo[3.1.0]hex-3-yl]naphthalene-2-carboxylic acid Lithium salt (Compound No. 15),
2-(4-Carboxypiperidin-1-yl)-6-[(1R,5S,6s)-6-{[(3,4-dichloro-5-methyl-1H-pyrrol-2-yl) carbonyl]amino}-3-azabicyclo[3.1.0]hex-3-yl]pyrimidine-4-carboxylic acid Lithium salt (Compound No. 16),
{4-[(1R,5S,6s)-6-{[(3,4-dichloro-5-methyl-1H-pyrrol-2-yl)carbonyl]amino}-3-azabicyclo[3.1.0]hex-3-yl]phenyl}acetic acid Lithium salt (Compound No. 17),
3-[(1R,5S,6s)-6-{[(3,4-dichloro-5-methyl-1H-pyrrol-2-yl)carbonyl]amino}-3-azabicyclo[3.1.0]hex-3-yl]benzoic acid Lithium salt (Compound No. 18),
2-(4-Aminopiperidin-1-yl)-6-[(1R,5S,6s)-6-{[(3,4-dichloro-5-methyl-1H-pyrrol-2-yl) carbonyl]amino}-3-azabicyclo[3.1.0]hex-3-yl]pyrimidine-4-carboxylic acid trifluoroacetate salt (Compound No. 19),
2-[(1R,5S,6s)-6-{[(3,4-dichloro-5-methyl-1H-pyrrol-2-yl)carbonyl]amino}-3-azabicyclo[3.1.0]hex-3-yl]-4-propyl-1,3-thiazole-5-carboxylic acid Lithium salt (Compound No. 20),
4-Benzyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 21),
2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxamide (Compound No. 22),
2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-4-carboxylic acid (Compound No. 23),
2-[(1R,5S,6s)-6-{[(3,4-dichloro-5-methyl-1H-pyrrol-2-yl)carbonyl]amino}-3-azabicyclo[3.1.0]hex-3-yl]-4-(methoxymethyl)-1,3-thiazole-5-carboxylic acid (Compound No. 24),
2-[(1R,5S,6s)-6-{[(3,4-dichloro-5-methyl-1H-pyrrol-2-yl)carbonyl]amino}-3-azabicyclo[3.1.0]hex-3-yl]-4-pentyl-1,3-thiazole-5-carboxylic acid (Compound No. 25),
2-[(1R,5S,6s)-6-{[(3,4-dichloro-5-methyl-1H-pyrrol-2-yl)carbonyl]amino}-3-azabicyclo[3.1.0]hex-3-yl]-4-phenyl-1,3-thiazole-5-carboxylic acid (Compound No. 26),
2-[(1R,5S,6s)-6-{[(3,4-dichloro-5-methyl-1H-pyrrol-2-yl)carbonyl]amino})-3-azabicyclo[3.1.0]hex-3-yl]-1,3-thiazole-4,5-dicarboxylic acid Sodium salt (Compound No. 27),
6-[(1R,5S,6s)-6-{[(3,4-dichloro-5-methyl-1H-pyrrol-2-yl)carbonyl]amino}-3-azabicyclo[3.1.0]hex-3-yl]-2-(piperidin-1-yl)pyrimidine-4-carboxylic acid Lithium salt (Compound No. 28),
2-{4-[(tert-butoxycarbonyl)amino]piperidin-1-yl]-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylic acid Lithium salt (Compound No. 29),
6-[(1R,5S,6s)-6-{[(3,4-dichloro-5-methyl-1H-pyrrol-2-yl)carbonyl]amino}-3-azabicyclo[3.1.0]hex-3-yl]-2-(pyrrolidin-1-yl)pyrimidine-4-carboxylic acid Lithium salt (Compound No. 30),
3,4-Dichloro-5-methyl-N-{(1R,5S,6s)-3-[2-(pyrrolidin-1-yl)-6-(pyrrolidin-1-ylcarbonyl)pyrimidin-4-yl]-3-azabicyclo[3.1.0]hex-6-yl}-1H-pyrrole-2-carboxamide (Compound No. 31),
N-[(1R,5S,6s)-3-(1,3-benzothiazol-2-yl)-3-azabicyclo[3.1.0]hex-6-yl]-3,4-dichloro-5-methyl-1H-pyrrole-2-carboxamide (Compound No. 32),
2-[(1R,5S,6s)-6-{[(3,4-dichloro-5-methyl-1H-pyrrol-2-yl)carbonyl]amino}-3-azabicyclo[3.1.0]hex-3-yl]4-[2-(methoxycarbonyl)benzyl]-1,3-thiazole-5-carboxylic acid (Compound No. 33),
2-[(1R,5S,6s)-6-{[(3,4-dichloro-5-methyl-1H-pyrrol-2-yl)carbonyl]amino-3-azabicyclo[3.1.0]hex-3-yl]-4-(phenoxymethyl)-1,3-thiazole-5-carboxylic acid (Compound No. 34),
4-(2-chloro-6-fluorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 35),
2-[(1R,5S,6s)-6-{[(3,4-dichloro-5-methyl-1H-pyrrol-2-yl)carbonyl]amino}-3-azabicyclo[3.1.0]hex-3-yl]-4-(2-phenylethyl)-1,3-thiazole-5-carboxylic acid (Compound No. 36),
2-[(1R,5S,6s)-6-{[(3,4-dichloro-5-methyl-1H-pyrrol-2-yl)carbonyl]amino}-3-azabicyclo[3.1.0]hex-3-yl]-4-(4-fluorobenzyl)-1,3-thiazole-5-carboxylic acid (Compound No. 37),
4-(3-Chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 38),
4-(Cyclopentylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 39),
4-[(Benzyloxy)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 40),
2-[(1R,5S,6s)-6-{[(3,4-dichloro-5-methyl-1H-pyrrol-2-yl)carbonyl]amino}-3-azabicyclo[3.1.0]hex-3-yl]-4-(3-fluorobenzyl)-1,3-thiazole-5-carboxylic acid (Compound No. 41),
4-Cyclohexyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 42),
4-(2-Cyclopentylethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 43),
2-[(1R,5S,6s)-6-{[(3,4-dichloro-5-methyl-1H-pyrrol-2-yl)carbonyl]amino}-3-azabicyclo[3.1.0]hex-3-yl]-4-(4-methoxybenzyl)-1,3-thiazole-5-carboxylic acid (Compound No. 44),
2-[(1R,5S,6s)-6-{[(3,4-dichloro-5-methyl-1H-pyrrol-2-yl)carbonyl]amino}-3-azabicyclo[3.1.0]hex-3-yl]-4-(4-fluorophenyl)-1,3-thiazole-5-carboxylic acid (Compound No. 45),
2-[(1R,5S,6s)-6-{[(3,4-dichloro-5-methyl-1H-pyrrol-2-yl)carbonyl]amino}-3-azabicyclo[3.1.0]hex-3-yl]-4-(3-fluorophenyl)-1,3-thiazole-5-carboxylic acid (Compound No. 46),
4-(Biphenyl-4-yl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 47),
4-Butyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 48),
2-[(1R,5S,6s)-6-{[(3,4-dichloro-5-methyl-1H-pyrrol-2-yl)carbonyl]amino}-3-azabicyclo[3.1.0]hex-3-yl]-4-(naphthalen-2-yl)-1,3-thiazole-5-carboxylic acid (Compound No. 49),
4-Cyclopentyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 50),
2-[(1R,5S,6s)-6-{[(3,4-dichloro-5-methyl-1H-pyrrol-2-yl)carbonyl]amino}-3-azabicyclo[3.1.0]hex-3-yl]-4-(3,5-dimethoxyphenyl)-1,3-thiazole-5-carboxylic acid (Compound No. 51),
2-[(1R,5S,6s)-6-{[(3,4-dichloro-5-methyl-1H-pyrrol-2-yl)carbonyl]amino}-3-azabicyclo[3.1.0]hex-3-yl]-4-[4-(trifluoromethoxy)phenyl]-1,3-thiazole-5-carboxylic acid (Compound No. 52),
2-[(1R,5S,6s)-6-{[(3,4-dichloro-5-methyl-1H-pyrrol-2-yl)carbonyl]amino}-3-azabicyclo[3.1.0]hex-3-yl]-4-(thiophen-2-yl)-1,3-thiazole-5-carboxylic acid (Compound No. 53),
2-[(1R,5S,6s)-6-{[(3,4-dichloro-5-methyl-1H-pyrrol-2-yl)carbonyl]amino}-3-azabicyclo[3.1.0]hex-3-yl]-4-(3,4-difluorobenzyl)-1,3-thiazole-5-carboxylic acid (Compound No. 54),
4-(1,3-Benzodioxol-5-ylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 55),
4-(2-Chloro-4-fluorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 56),
4-(2-Bromobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 57),
2-[(1R,5S,6s)-6-{[(3,4-dichloro-5-methyl-1H-pyrrol-2-yl)carbonyl]amino}-3-azabicyclo[3.1.0]hex-3-yl]-4-(naphthalen-2-ylmethyl)-1,3-thiazole-5-carboxylic acid (Compound No. 58),
4-(2-Chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 59),
2-[(1R,5S,6s)-6-{[(3,4-dichloro-5-methyl-1H-pyrrol-2-yl)carbonyl]amino}-3-azabicyclo[3.1.0]hex-3-yl]-4-(2-methylbenzyl)-1,3-thiazole-5-carboxylic acid (Compound No. 60),
4-(3-Bromobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 61),
2-[(1R,5S,6s)-6-{[(3,4-dichloro-5-methyl-1H-pyrrol-2-yl)carbonyl]amino}-3-azabicyclo[3.1.0]hex-3-yl]-4-(2-methoxybenzyl)-1,3-thiazole-5-carboxylic acid (Compound No. 62),
2-[(1R,5S,6s)-6-{[(3,4-dichloro-5-methyl-1H-pyrrol-2-yl)carbonyl]amino}-3-azabicyclo[3.1.0]hex-3-yl]-4-(naphthalen-1-ylmethyl)-1,3-thiazole-5-carboxylic acid (Compound No. 63),
4-(2-Chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 64),
4-(3-Chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 65),
4-(4-Bromo-2-methylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 66),
4-(3-Chloro-4-fluorophenyl)-2-{[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 67),
4-(4-Chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound No. 68),
2-[(1R,5S,6s)-6-{[(3,4-dichloro-5-methyl-1H-pyrrol-2-yl)carbonyl]amino}-3-azabicyclo[3.1.0]hex-3-yl]-4-[4-(trifluoromethyl)benzyl]-1,3-thiazole-5-carboxylic acid (Compound No. 69),
3,4-Dichloro-5-methyl-N-{[(1R,5S,6s)-3-[4-(naphthalen-2-ylmethyl)-1,3-thiazol-2-yl]-3-azabicyclo[3.1.0]hex-6-yl}-1H-pyrrole-2-carboxamide (Compound No. 70),
4-(2,4-Dichlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 71),
4-(4-Butoxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 72),
2-[(1R,5S,6s)-6-{[(3,4-dichloro-5-methyl-1H-pyrrol-2-yl)carbonyl]amino}-3-azabicyclo[3.1.0]hex-3-yl]-4-(4-ethoxybenzyl)-1,3-thiazole-5-carboxylic acid Lithium salt (Compound No. 73),
4-(Biphenyl-4-ylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 74),
4-(Biphenyl-4-yl-methyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Sodium salt (Compound No. 74a)
2-[(1R,5S,6s)-6-{[(3,4-dichloro-5-methyl-1H-pyrrol-2-yl)carbonyl]amino}-3-azabicyclo[3.1.0]hex-3-yl]-4-(5-methylthiophen-2-yl)-1,3-thiazole-5-carboxylic acid Lithium salt (Compound No. 75),
4-[3,5-bis(trifluoromethyl)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 76),
4-[(3′-Chlorobiphenyl-3-yl)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 77),
2-[(1R,5S,6s)-6-{[(3,4-dichloro-5-methyl-1H-pyrrol-2-yl)carbonyl]amino}-3-azabicyclo[3.1.0]hex-3-yl]-4-[(3′,4′-difluorobiphenyl-3-yl)methyl]-1,3-thiazole-5-carboxylic acid Lithium salt (Compound No. 78),
4-(4-Acetylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 79),
4-(4-tert-Butylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 80),
2-[(1R,5S,6s)-6-{[(3,4-dichloro-5-methyl-1H-pyrrol-2-yl)carbonyl]amino}-3-azabicyclo[3.1.0]hex-3-yl]-4-(4-phenoxyphenyl)-1,3-thiazole-5-carboxylic acid Lithium salt (Compound No. 81),
2-[(1R,5S,6s)-6-{[(3,4-dichloro-5-methyl-1H-pyrrol-2-yl)carbonyl]amino}-3-azabicyclo[3.1.0]hex-3-yl]-4-(3-methoxybenzyl)-1,3-thiazole-5-carboxylic acid Lithium salt (Compound No. 82),
4-(4-Carboxyphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 83),
4-(3-Carboxyphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 84),
2-[(1R,5S,6s)-6-{[(3,4-dichloro-5-methyl-1H-pyrrol-2-yl)carbonyl]amino}-3-azabicyclo[3.1.0]hex-3-yl]4-[2-fluoro-5-(trifluoromethyl)benzyl]-1,3-thiazole-5-carboxylic 315 acid Lithium salt (Compound No. 85),
4-(4-Chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 86),
4-(3-Carboxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 87),
2-[(1R,5S,6s)-6-{[(3,4-dichloro-5-methyl-1H-pyrrol-2-yl)carbonyl]amino}-3-azabicyclo[3.1.0]hex-3-yl]-4-(2-ethoxybenzyl)-1,3-thiazole-5-carboxylic acid Lithium salt (Compound No. 88),
4-(4-Carboxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 89),
4-[4-(Acetylamino)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 90),
2-[(1R,5S,6s)-6-{[(3,4-dichloro-5-methyl-1H-pyrrol-2-yl)carbonyl]amino}-3-azabicyclo[3.1.0]hex-3-yl]-4-(hydroxymethyl)-1,3-thiazole-5-carboxylic acid Lithium salt (Compound No. 91),
2-[(1R,5S,6s)-6-{[(3,4-dichloro-5-methyl-1H-pyrrol-2-yl)carbonyl]amino}-3-azabicyclo[3.1.0]hex-3-yl]-4-[4-(ethylcarbamoyl)benzyl]-1,3-thiazole-5-carboxylic acid Lithium salt (Compound No. 92),
4-(4-Carbamoylbenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound No. 93),
2-[(1R,5S,6s)-6-{[(3,4-dichloro-5-methyl-1H-pyrrol-2-yl)carbonyl]amino}-3-azabicyclo[3.1.0]hex-3-yl]-4-[4-(methylcarbamoyl)benzyl]-1,3-thiazole-5-carboxylic acid (Compound No. 94),
2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-oxazole-5-carboxylic acid Lithium salt (Compound No. 95),
2-[(1R,5S,6s)-6-{[(3,4-dichloro-5-methyl-1H-pyrrol-2-yl)carbonyl]amino}-3-azabicyclo[3.1.0]hex-3-yl]-4-[3-(trifluoromethyl)benzyl]-1,3-oxazole-5-carboxylic acid (Compound No. 96),
4-(4-Chlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-oxazole-5-carboxylic acid (Compound No. 97),
2-[(1R,5S,6s)-6-{[(3,4-dichloro-5-methyl-1H-pyrrol-2-yl)carbonyl]amino}-3-azabicyclo[3.1.0]hex-3-yl]-4-(4-ethoxybenzyl)-1,3-oxazole-5-carboxylic acid (Compound No. 98),
2-[(1R,5S,6s)-6-{[(3,4-dichloro-5-methyl-1H-pyrrol-2-yl)carbonyl]amino}-3-azabicyclo[3.1.0]hex-3-yl]-4-(pyridin-3-yl)-1,3-thiazole-5-carboxylic acid (Compound No. 99),
2-[(1R,5S,6s)-6-{[(3,4-dichloro-5-methyl-1H-pyrrol-2-yl)carbonyl]amino}-3-azabicyclo[3.1.0]hex-3-yl]-4-(pyridin-4-yl)-1,3-thiazole-5-carboxylic acid (Compound No. 100),
2-[(1R,5S,6s)-6-{[(3,4-dichloro-5-methyl-1H-pyrrol-2-yl)carbonyl]amino}-3-azabicyclo[3.1.0]hex-3-yl]-4-(trifluoromethyl)-1,3-thiazole-5-carboxylic acid (Compound no. 101),
2-[(1R,5S,6s)-6-{[(3,4-dichloro-5-methyl-1H-pyrrol-2-yl)carbonyl]amino}-3-azabicyclo[3.1.0]hex-3-yl]4-[2-(pyridin-4-yl)ethyl]-1,3-thiazole-5-carboxylic acid (Compound no. 102),
N-[3-({2-[(1R,5S,6s)-6-{[(3,4-dichloro-5-methyl-1H-pyrrol-2-yl)carbonyl]amino}-3-azabicyclo[3.1.0]hex-3-yl]-5-(ethoxycarbonyl)-1,3-thiazol-4-yl]methyl)phenyl]glycine (Compound no. 103),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(hydroxymethyl)-1,3-thiazole-5-carboxylate (Compound no. 104),
4-(5-Acetylthiophen-2-yl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 105),
4-({2-[(1R,5S,6s)-6-{[(3,4-dichloro-5-methyl-1H-pyrrol-2-yl)carbonyl]amino}-3-azabicyclo[3.1.0]hex-3-yl]-5-(ethoxycarbonyl)-1,3-thiazol-4-yl}methyl)benzoic acid Lithium salt (Compound no. 106),
4-[3-(Acetylamino)benzyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 107),
2-[(1R,5S,6s)-6-{[(3,4-dichloro-5-methyl-1H-pyrrol-2-yl)carbonyl]amino}-3-azabicyclo[3.1.0]hex-3-yl]-4-[3-(trifluoromethyl)benzyl]-1,3-thiazole-5-carboxylic acid Lithium salt (Compound no. 108),
2-[(1R,5S,6s)-6-{[(3,4-dichloro-5-methyl-1H-pyrrol-2-yl)carbonyl]amino}-3-azabicyclo[3.1.0]hex-3-yl]-4-{3-[(ethylcarbamoyl)amino]benzyl}-1,3-thiazole-5-carboxylic acid Lithium salt (Compound no. 109),
4-(2-Carboxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 110),
4-(2-Carboxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 111),
2-({5-(tert-butoxycarbonyl)-2-[(1R,5S,6 s)-6-{[(3,4-dichloro-5-methyl-1H-pyrrol-2-yl)carbonyl]amino}-3-azabicyclo[3.1.0]hex-3-yl]-1,3-thiazol-4-yl]methyl)benzoic acid (Compound no. 112),
2-[(1R,5S,6s)-6-{[(3,4-dichloro-5-methyl-1H-pyrrol-2-yl)carbonyl]amino}-3-azabicyclo[3.1.0]hex-3-yl]4-[2-(ethylamino)-2-oxoethyl]-1,3-thiazole-5-carboxylic acid (Compound no. 113),
4-(2-{4-[(tert-Butoxycarbonyl)amino]piperidin-1-yl}-2-oxoethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 114),
4-[(3′-Carboxybiphenyl-3-yl)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 115),
2-[(1R,5S,6s)-6-{[(3,4-dichloro-5-methyl-1H-pyrrol-2-yl)carbonyl)amino}-3-azabicyclo[3.1.0]hex-3-yl]-4-[3-(6-fluoropyridin-3-yl)benzyl]-1,3-thiazole-5-carboxylic acid (Compound no. 116),
2-[(1R,5S,6s)-6-{[(3,4-dichloro-5-methyl-1H-pyrrol-2-yl)carbonyl]amino}-3-azabicyclo[3.1.0]hex-3-yl]-4-{4-[(4-hydroxypiperidin-1-yl)carbonyl]benzyl]-1,3-thiazole-5-carboxylic acid (Compound no. 117),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-{-4-[(ethylcarbamoyl)amino]benzyl}-1,3-thiazole-5-carboxylate (Compound no. 118),
4-[2-(Cyclopropylamino)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 119),
4-[2-(Cyclopentylamino)-2-oxoethyl]-2-{[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 120),
4-{2-[(Carboxymethyl)amino]-2-oxoethyl]-2-[(1R,5S,6s)-6-1 [(3,4-dichloro-5-methyl-1H-pyrrol-2-yl)carbonyl]amino}-3-azabicyclo[3.1.0]hex-3-yl]-1,3-thiazole-5-carboxylic acid (Compound no. 121),
N-({2-[(1R,5S,6s)-6-{[(3,4-dichloro-5-methyl-1H-pyrrol-2-yl)carbonyl]amino}-3-azabicyclo[3.1.0]hex-3-yl]-5-(methoxycarbonyl)-1,3-thiazol-4-yl▾acetyl)glycine Lithium salt (Compound no. 122),
4-[2-(Cyclobutylamino)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 123),
2-[(1R,5S,6s)-6-{[(3,4-dichloro-5-methyl-1H-pyrrol-2-yl)carbonyl]amino}-3-azabicyclo[3.1.0]hex-3-yl]-4-(2-oxo-2-{[3-(trifluoromethyl)phenyl]amino}ethyl)-1,3-471 thiazole-5-carboxylic acid Lithium salt (Compound no. 124),
4-{2-[(3-Chlorophenyl)amino]-2-oxoethyl}-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid Lithium salt (Compound no. 125),
2-[(1R,5S,6s)-6-{[(3,4-dichloro-5-methyl-1H-pyrrol-2-yl)carbonyl]amino}-3-azabicyclo[3.1.0]hex-3-yl]4-[2-(4-hydroxypiperidin-1-yl)-2-oxoethyl]-1,3-thiazole-5-carboxylic acid (Compound no. 126),
2-[(1R,5S,6s)-6-{[(3,4-dichloro-5-methyl-1H-pyrrol-2-yl)carbonyl]amino}-3-azabicyclo[3.1.0]hex-3-yl]4-[2-(morpholin-4-yl)-2-oxoethyl]-1,3-thiazole-5-carboxylic acid (Compound no. 127),
2-[(1R,5S,6s)-6-{[(3,4-dichloro-5-methyl-1H-pyrrol-2-yl)carbonyl]amino}-3-azabicyclo[3.1.0]hex-3-yl]4-[2-(methoxyamino)-2-oxoethyl]-1,3-thiazole-5-carboxylic acid (Compound no. 128),
2-[(1R,5S,6s)-6-{[(3,4-dichloro-5-methyl-1H-pyrrol-2-yl)carbonyl]amino}-3-azabicyclo[3.1.0]hex-3-yl]4-[2-oxo-2-(pyridin-3-ylamino)ethyl]-1,3-thiazole-5-carboxylic acid (Compound no. 129),
4-[2-(Azetidin-1-yl)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 130),
2-[(1R,5S,6s)-6-{[(3,4-dichloro-5-methyl-1H-pyrrol-2-yl)carbonyl]amino}-3-azabicyclo[3.1.0]hex-3-yl]4-[2-(4-methylpiperazin-1-yl)-2-oxoethyl]-1,3-thiazole-5-carboxylic acid (Compound no. 131),
4-{2-[4-(tert-Butoxycarbonyl)piperazin-1-yl]-2-oxoethyl}-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 132),
2-[(1R,5S,6s)-6-{[(3,4-dichloro-5-methyl-1H-pyrrol-2-yl)carbonyl]amino}-3-azabicyclo[3.1.0]hex-3-yl]-4-(2-oxo-2-{[4-(4H-1,2,4-triazol-4-yl)phenyl]amino}ethyl)-1,3-thiazole-5-carboxylic acid (Compound no. 133),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(4-methoxybenzyl)amino]-2-oxoethyl}-1,3-thiazole-5-carboxylic acid (Compound no. 134),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(4-methyl-1,3-thiazol-2-yl)amino]-2-oxoethyl}-1,3-thiazole-5-carboxylic acid (Compound no. 135),
2-[(1R,5S,6s)-6-{[(3,4-dichloro-5-methyl-1H-pyrrol-2-yl)carbonyl]amino}-3-azabicyclo[3.1.0]hex-3-yl]-4-(2-oxo-2-{[4-(trifluoromethyl)benzyl]amino}ethyl)-1,3-thiazole-5-carboxylic acid (Compound no. 136),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(3-fluorophenyl)amino]-2-oxoethyl}-1,3-thiazole-5-carboxylic acid (Compound no. 137),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(4-ethylphenyl)amino]-2-oxoethyl}-1,3-thiazole-5-carboxylic acid (Compound no. 138),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(3-methylbenzyl)amino]-2-oxoethyl}-1,3-thiazole-5-carboxylic acid (Compound no. 139),
2-[(1R,5S,6s)-6-{[(3,4-dichloro-5-methyl-1H-pyrrol-2-yl)carbonyl]amino}-3-azabicyclo[3.1.0]hex-3-yl]4-[2-oxo-2-(piperidin-1-yDethyl]-1,3-thiazole-5-carboxylic acid (Compound no. 140),
4-(2-{3-[(tert-Butoxycarbonyl)amino]pyrrolidin-1-yl]-2-oxoethyl}-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 141),
4-[2-(3-Aminopyrrolidin-1-yl)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 142),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(1-ethyl-1H-pyrazol-5-yl)amino]-2-oxoethyl}-1,3-thiazole-5-carboxylic acid (Compound no. 143),
4-[2-(4-Aminopiperidin-1-yl)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 144),
2-[(1R,5S,6s)-6-[{3,4-dichloro-5-methyl-1H-pyrrol-2-yl)carbonyl]amino}-3-azabicyclo[3.1.0]hex-3-yl]4-[2-oxo-2-(piperazin-1-yl)ethyl]-1,3-thiazole-5-carboxylic acid (Compound no. 145),
2-[(1R,5S,6s)-6-{[(3,4-dichloro-5-methyl-1H-pyrrol-2-yl)carbonyl]amino}-3-azabicyclo[3.1.0]hex-3-yl]4-[2-oxo-2-(1,3-thiazol-2-yl)ethyl]-1,3-thiazole-5-carboxylic acid (Compound no. 146),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(3-methyl-1,2-oxazol-5-yl)amino]-2-oxoethyl}-1,3-thiazole-5-carboxylic acid (Compound no. 147),
4-[2-(1H-Benzimidazol-2-ylamino)-2-oxoethyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 148),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(2-hydroxyethyl)amino]-2-oxoethyl}-1,3-thiazole-5-carboxylic acid (Compound no. 149),
4-{2-[(3-Chlorobenzyl)amino}-2-oxoethyl}-2-[(1R,5S,6s)-6-1 [(3,4-dichloro-5-methyl-1H-pyrrol-2-yl)carbonyl]amino}-3-azabicyclo[3.1.0]hex-3-yl]-1,3-thiazole-5-carboxylic acid (Compound no. 150),
2-[(1R,5S,6s)-6-{[(3,4-dichloro-5-methyl-1H-pyrrol-2-yl)carbonyl]amino}-3-azabicyclo[3.1.0]hex-3-yl]-4-{2-[(1-hydroxy-2-methylpropan-2-yl)amino]-2-oxoethyl}-1,3-thiazole-5-carboxylic acid (Compound no. 151),
4-{2-[(4-Carboxyphenyl)amino]-2-oxoethyl}-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylic acid (Compound no. 152),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxylate (Compound no. 153),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 154),
Methyl 2-chloro-6-[(1R,5S,6s)-6-{[(3,4-dichloro-5-methyl-1H-pyrrol-2-yl)carbonyl]amino}-3-azabicyclo[3.1.0]hex-3-yl]pyridine-4-carboxylate (Compound no. 155),
Methyl 6-[(1R,5S,6s)-6-{[(3,4-dichloro-5-methyl-1H-pyrrol-2-yl)carbonyl]amino}-3-azabicyclo[3.1.0]hex-3-yl]pyridine-3-carboxylate (Compound no. 156),
Methyl 6-[(1R,5S,6s)-6-{[(3,4-dichloro-5-methyl-1H-pyrrol-2-yl)carbonyl]amino}-3-azabicyclo[3.1.0]hex-3-yl]-2-(morpholin-4-yl)pyrimidine-4-carboxylate (Compound no. 157),
Methyl 2-[(cyclopropylmethyl)amino]-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylate (Compound no. 158),
Methyl 2-{4-[(tert-butoxycarbonyl)amino]piperidin-1-yl}-6-[(1R,5S,6s)-6-{[(3,4-dichloro-5-methyl-1H-pyrrol-2-yl)carbonyl]amino}-3-azabicyclo[3.1.0]hex-3-yl]pyrimidine-4-carboxylate (Compound no. 159),
tert-Butyl 2-[(1R,5S,6s)-6-{[(3,4-dichloro-5-methyl-1H-pyrrol-2-yl)carbonyl]amino}-3-azabicyclo[3.1.0]hex-3-yl]4-[2-(methoxycarbonyl)benzyl]-1,3-thiazole-5-carboxylate (Compound no. 160),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(methoxymethyl)-1,3-thiazole-5-carboxylate (Compound no. 161),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(phenoxymethyl)-1,3-thiazole-5-carboxylate (Compound no. 162),
Ethyl 4-(cyclopentylmethyl)-2-{[(1R,5S,6s)-6-[(3,4-dichloro-5-methyl-1H-pyrrol-2-yl)carbonyl]amino}-3-azabicyclo[3.1.0]hex-3-yl]-1,3-thiazole-5-carboxylate (Compound no. 163),
Ethyl 4-[(benzyloxy)methyl]-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 164),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(3-fluorobenzyl)-1,3-thiazole-5-carboxylate (Compound no. 165),
Ethyl 4-(2-cyclopentylethyl)-2-{[(1R,5S,6s)-6-1 [(3,4-dichloro-5-methyl-1H-pyrrol-2-yl)carbonyl]amino}-3-azabicyclo[3.1.0]hex-3-yl]-1,3-thiazole-5-carboxylate (Compound no. 166),
Ethyl 4-cyclohexyl-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 167),
Ethyl 4-(2-chlorophenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 168),
Ethyl 4-(4-bromo-2-methylphenyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 169),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-[3-(6-fluoropyridin-3-yl)benzyl]-1,3-thiazole-5-carboxylate (Compound no. 170),
Ethyl 4-(2,4-dichlorobenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 171),
Ethyl 4-(4-butoxybenzyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 172),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(3-methoxybenzyl)-1,3-thiazole-5-carboxylate (Compound no. 173),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(4-ethoxybenzyl)-1,3-thiazole-5-carboxylate (Compound no. 174),
Ethyl 4-(biphenyl-4-ylmethyl)-2-[(1R,5S,6s)-6-{[(3,4-dichloro-5-methyl-1H-pyrrol-2-yl)carbonyl]amino}-3-azabicyclo[3.1.0]hex-3-yl]-1,3-thiazole-5-carboxylate (Compound no. 175),
Ethyl 2-[(1R,5S,6s)-6-{[(3,4-dichloro-5-methyl-1H-pyrrol-2-yl)carbonyl]amino}-3-azabicyclo[3.1.0]hex-3-yl]-4-(2,5-dimethoxybenzyl)-1,3-thiazole-5-carboxylate (Compound no. 176),
tert-Butyl 2-[(1R,5S,6s)-6-{[(3,4-dichloro-5-methyl-1H-pyrrol-2-yl)carbonyl]amino}-3-azabicyclo[3.1.0]hex-3-yl]-4-methyl-1,3-thiazole-5-carboxylate (Compound no. 177),
Ethyl 2-[(1R,5S,6s)-6-[(3,4-dichloro-5-methyl-1H-pyrrol-2-yl)carbonyl]amino)-3-azabicyclo[3.1.0]hex-3-yl]-4-(pyridin-4-yl)-1,3-thiazole-5-carboxylate (Compound no. 178) and
{2-[(1R,5S,6s)-6-1 [(3,4-dichloro-5-methyl-1H-pyrrol-2-yl)carbonyl]amino}-3-azabicyclo[3.1.0]hex-3-yl]-5-(methoxycarbonyl)-1,3-thiazol-4-yl]acetic acid (Compound no. 179).

3. A pharmaceutical composition comprising therapeutically effective amount compound of claim 1 or 2 together with one or more pharmaceutically acceptable carrier, excipient or diluent.

4. The pharmaceutical composition of claim 3 further comprising one or more additional active ingredient selected from (i) protein synthesis inhibitors (ii) aminoglycosides (iii) cell wall synthesis inhibitors (iv) RNA and DNA synthesis inhibitors (v) fatty acid synthesis inhibitors or other therapeutic agents to treat, prevent or inhibit nosocomial and community acquired bacterial infection or a associated disease, disorder or infection.

5. A method for treating or preventing a condition caused by or contributed by bacterial infection comprising administering to a mammal in need thereof therapeutically effective amount of compound of claim 1 or 2.

6. A method for treating or preventing a condition caused by or contributed by bacterial infection comprising administering to a mammal in need thereof therapeutically effective amount of pharmaceutical composition of claim 3 or 4.

7. The method of claim 5 or 6, wherein the condition is selected from community acquired pneumonia, upper or lower respiratory tract infections, skin or soft tissue infections, hospital acquired lung infections, urinary tract infections, intra-abdominal infections, enterococci infections, bacteraemia infections with known or suspected endocarditis, nosocomial bone or joint infections, acne vulgaris, mastitis, catheter infection, foreign body, prosthesis infections or peptic ulcer disease.

8. The method of claim 5 or 6, wherein the bacterial infection is caused by Gram-positive, Gram-negative or anaerobic bacteria.

9. The method of claim 8, wherein the Gram-positive, Gram-negative or anaerobic bacteria is selected from Staphylococci, Streptococci, Enterococci, Haemophilus, Moraxella spp., Chlamydia spp., Mycoplasm, Legionella spp., Mycobacterium, Helicobacter, Clostridium, Bacteroides, Corynebacterium, Bacillus and Enterobactericeae.

10. The method of claim 9, wherein the bacterium is cocci.

11. The method of claim 10, wherein the cocci is drug resistant.

12. A process for preparing a compound of Formula VIII 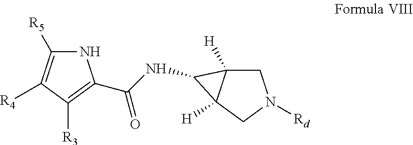
which comprises the steps of:
a) N-deprotection of compound of Formula II 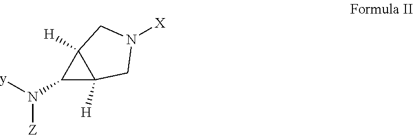
to give a compound of Formula III 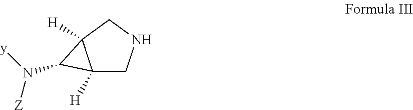
b) reacting compound of Formula III with a compound of Formula IV
L-R_{d} Formula IV
to give a compound of Formula V 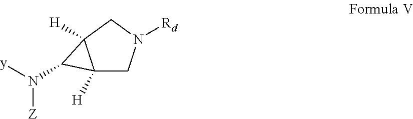
c) N-deprotection of compound of Formula V to give a compound of Formula VI 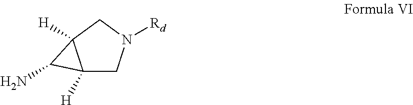
d) coupling of compound of Formula VI with a compound of Formula VII 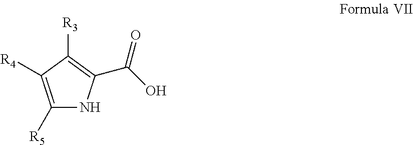
to give a compound of Formula VIII
OR
a) deprotection of a compound of Formula II to give a compound of Formula IX 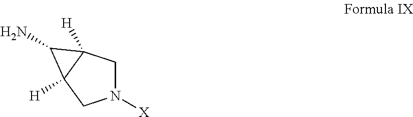
b) coupling of compound of Formula IX with a compound of Formula VII to give a compound of Formula X 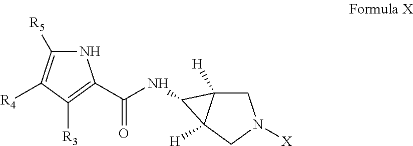
c) N-deprotection of compound of Formula X to give a compound of Formula XI 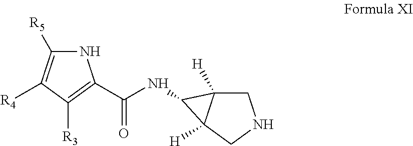
d) reacting compound of Formula III with a compound of Formula IV to give a compound of Formula VIII
wherein
R₃, R₄, R₅ are independently hydrogen, alkyl, alkenyl, alkynyl, halogen, cyano, amino, hydroxy, alkoxy, carbonyl, thiocarbonyl, oxo, cycloalkyl, aryl, heteroaryl, heterocyclyl, arylalkyl, heteroarylalkyl, cycloalkylalkyl or heterocyclylalkyl or R₃ and R₄ or R₄ and R₅ taken together with the carbon atoms to which they are attached can form an aromatic or non-aromatic ring, which optionally contain heteroatom selected from N, O and S;
R_{d} is (un)substituted aryl or heteroaryl;
y is H or an amine protecting group selected from t-butoxycarbonyl (t-BOC), 9-fluorenylmethoxycarbonyl (Fmoc), phthalimide, trityl, allyloxycarbonyl, trifluoroacetamide, tosyl, benzyl, benzyloxycarbonyl or pyridine-2-sulfonyl;
Z is an amine protecting group selected from t-butoxycarbonyl (t-BOC), 9-fluorenylmethoxycarbonyl (Fmoc), phthalimide, trityl, allyloxycarbonyl, trifluoroacetamide, tosyl, benzyl, benzyloxycarbonyl or pyridine-2-sulfonyl;
X is an amine protecting group selected from benzyl, benzyloxycarbonyl, trifluoroacetyl, allyloxycarbonyl, t-butoxycarbonyl (t-BOC) or pyridine-2-sulfonyl;
L is a leaving group selected from chloro, bromo or tosyl.

13. A process for preparing a compound of Formula XII 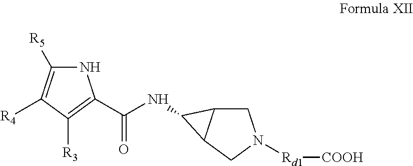
which comprises the steps of:
a) hydrolyzing the compound of Formula VIII 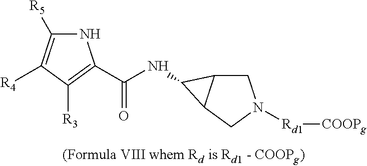
to give a compound of Formula XII
wherein
R₃, R₄, R₅ are independently hydrogen, alkyl, alkenyl, alkynyl, halogen, cyano, amino, hydroxy, alkoxy, carbonyl, thiocarbonyl, oxo, cycloalkyl, aryl, heteroaryl, heterocyclyl, arylalkyl, heteroarylalkyl, cycloalkylalkyl or heterocyclylalkyl or R₃ and R₄ or R₄ and R₅ taken together with the carbon atoms to which they are attached can form an aromatic or non-aromatic ring, which optionally contain heteroatom selected from N, O and S;
R_{d1} is aryl or heteroaryl;
P_{g} is an alkyl group selected from methyl, ethyl or t-butyl.

14. A process for preparing a compound of Formula XV 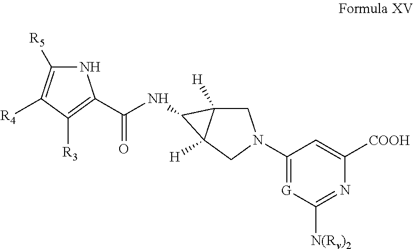
which comprises the steps of:
a) reacting the compound of Formula VIII 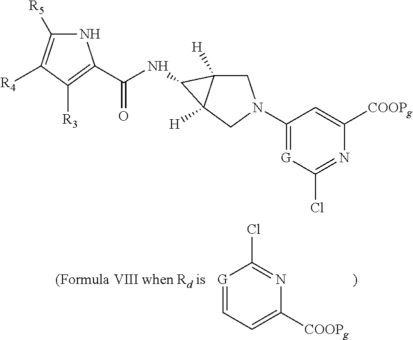
with a compound of Formula XXIII
NH(Rᵥ)₂ Formula XXIII
to give a compound of Formula XIV 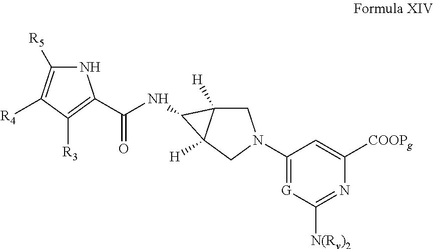
b) deprotection of compound of Formula XIV to give a compound of Formula XV wherein
R₃, R₄, R₅ are independently hydrogen, alkyl, alkenyl, alkynyl, halogen, cyano, amino, hydroxy, alkoxy, carbonyl, thiocarbonyl, oxo, cycloalkyl, aryl, heteroaryl, heterocyclyl, arylalkyl, heteroarylalkyl, cycloalkylalkyl or heterocyclylalkyl or R₃ and R₄ or R₄ and R₅ taken together with the carbon atoms to which they are attached can form an aromatic or non-aromatic ring, which optionally contain heteroatom selected from N, O and S;
G is N or CH;
Rᵥ is hydrogen or alkyl or two Rᵥ along with the N to which they are attached join together to form a heterocyclic ring optionally containing heteroatoms O, N or S;
P_{g} is an alkyl group selected from methyl, ethyl or t-butyl.

15. A process for preparing a compound of Formula XVI 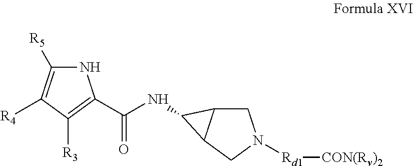
which comprises the steps of:
a) coupling of compound of Formula XII 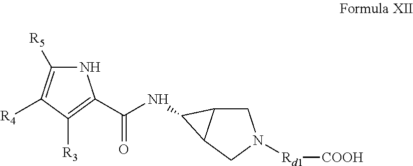
with a compound of Formula XXIII
NH(Rᵥ)₂ Formula XXIII
to give a compound of Formula XVI.
wherein
R₃, R₄, R₅ are independently hydrogen, alkyl, alkenyl, alkynyl, halogen, cyano, amino, hydroxy, alkoxy, carbonyl, thiocarbonyl, oxo, cycloalkyl, aryl, heteroaryl, heterocyclyl, arylalkyl, heteroarylalkyl, cycloalkylalkyl or heterocyclylalkyl or R₃ and R₄ or R₄ and R₅ taken together with the carbon atoms to which they are attached can form an aromatic or non-aromatic ring, which optionally contain heteroatom selected from N, O and S;
R_{d1} is aryl or heteroaryl;
Rᵥ is hydrogen or alkyl or two Rᵥ along with the N to which they are attached join together to form a heterocyclic ring optionally containing heteroatoms O, N or S.

16. A process for preparing a compound of Formula XXI 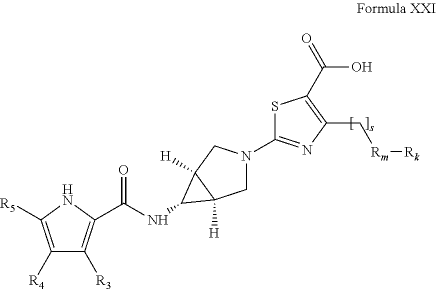
which comprises the steps of:
a) coupling the compound of Formula V 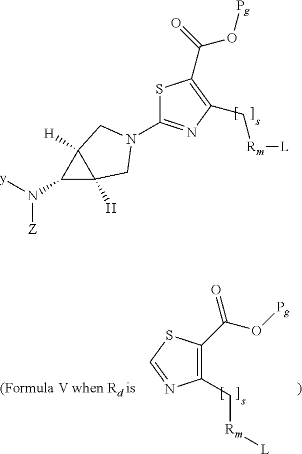
with a compound of Formula XVII
Rₖ—B(OH)₂ Formula XVII
to give a compound of Formula XVIII. 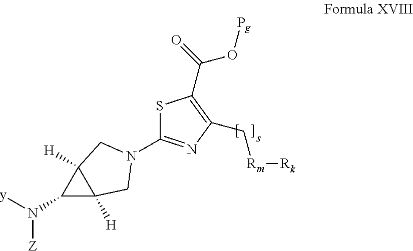
b) N-deprotection of the compound of Formula XVIII to give a compound of Formula XIX 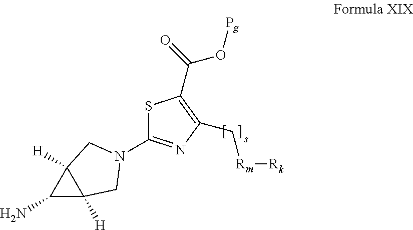
c) coupling of the compound of Formula XIX with a compound of Formula VII to give a compound of Formula XX 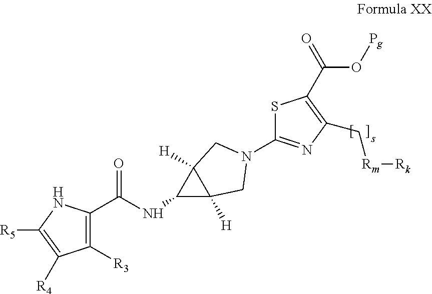
d) hydrolyzing the compound of Formula XX to give a compound of Formula XXI
wherein
wherein
R₃, R₄, R₅ are independently hydrogen, alkyl, alkenyl, alkynyl, halogen, cyano, amino, hydroxy, alkoxy, carbonyl, thiocarbonyl, oxo, cycloalkyl, aryl, heteroaryl, heterocyclyl, arylalkyl, heteroarylalkyl, cycloalkylalkyl or heterocyclylalkyl or R₃ and R₄ or R₄ and R₅ taken together with the carbon atoms to which they are attached can form an aromatic or non-aromatic ring, which optionally contain heteroatom selected from N, O and S;
Rₘ and Rₖ are aryl or heteroaryl;
s is 0 or 1;
L is a leaving group selected from chloro, bromo or tosyl;
y is H or an amine protecting group selected from t-butoxycarbonyl (t-BOC), 9-fluorenylmethoxycarbonyl (Fmoc), phthalimide, trityl, allyloxycarbonyl, trifluoroacetamide, tosyl, benzyl, benzyloxycarbonyl or pyridine-2-sulfonyl;
Z is an amine protecting group selected from t-butoxycarbonyl (t-BOC), 9-fluorenylmethoxycarbonyl (Fmoc), phthalimide, trityl, allyloxycarbonyl, trifluoroacetamide, tosyl, benzyl, benzyloxycarbonyl or pyridine-2-sulfonyl;
P_{g} is an alkyl group selected from methyl, ethyl or t-butyl.

17. A process for preparing a compound of Formula XXV 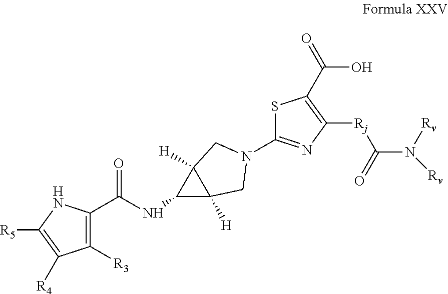
which comprises the steps of:
a) hydrolyzing the compound of Formula VIII 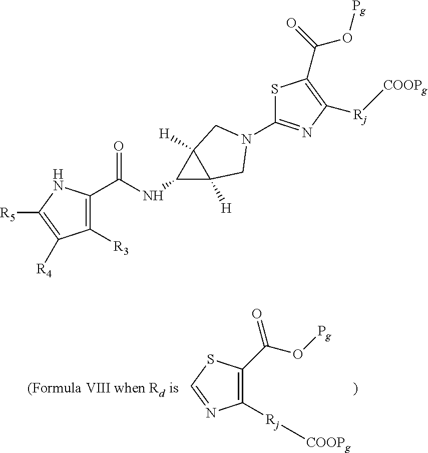
to give a compound of Formula XXII. 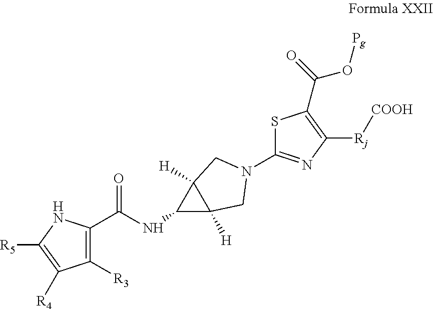
b) coupling the compound of Formula XXII with a compound of Formula XXIII
NH(Rᵥ)₂ Formula XXIII
to give a compound of Formula XXIV 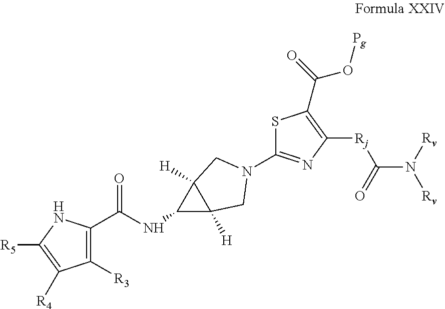
c) hydrolyzing the compound of Formula XXIV to give a compound of Formula XXV
wherein
R₃, R₄, R₅ are independently hydrogen, alkyl, alkenyl, alkynyl, halogen, cyano, amino, hydroxy, alkoxy, carbonyl, thiocarbonyl, oxo, cycloalkyl, aryl, heteroaryl, heterocyclyl, arylalkyl, heteroarylalkyl, cycloalkylalkyl or heterocyclylalkyl or R₃ and R₄ or R₄ and R₅ taken together with the carbon atoms to which they are attached can form an aromatic or non-aromatic ring, which optionally contain heteroatom selected from N, O and S;
Rⱼ is methylene or benzylene;
Rᵥ is hydrogen or alkyl or two Rᵥ along with the N to which they are attached join together to form a heterocyclic ring optionally containing heteroatoms O, N or S;
P_{g} is an alkyl group selected from methyl, ethyl or t-butyl.

18. A process for preparing a compound of Formula XXVIII, XXX and XXXII 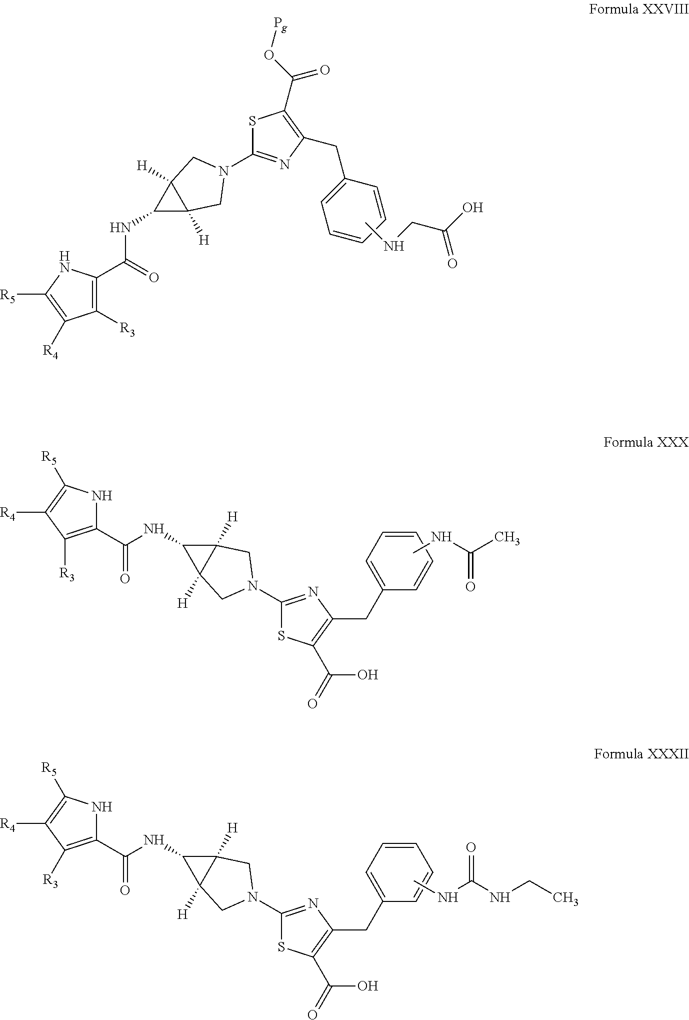
which comprises the steps of:
a) reducing the compound of Formula VIII 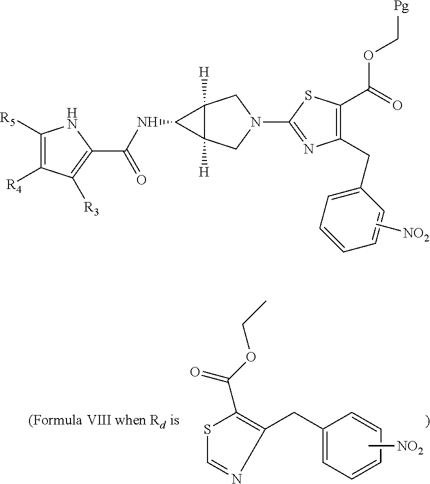
to give a compound of Formula XXVI 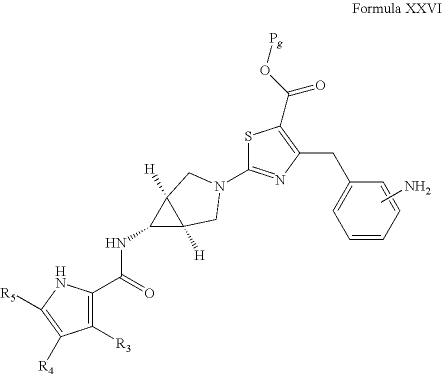
b) reacting the compound of Formula XXVI with methyl bromoacetate to give a compound of Formula XXVII 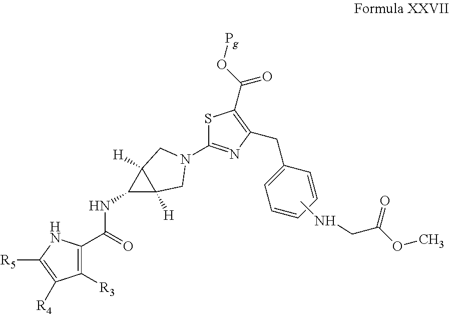
c) hydrolyzing the compound of Formula XXVII to give a compound of Formula XXVIII
OR
a) reducing the compound of Formula VIII to a compound of Formula XXVI
b) reacting the compound of Formula XXVI with acetyl chloride to give a compound of Formula XXIX 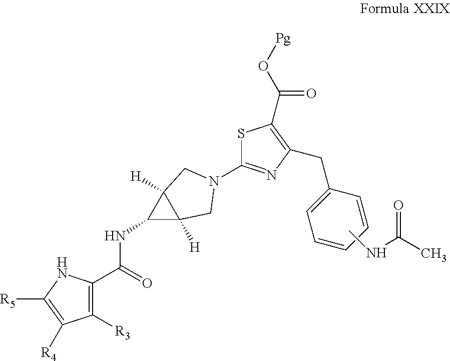
c) hydrolyzing the compound of Formula XXIX to give a compound of Formula XXX
OR
a) reducing the compound of Formula VIII to a compound of Formula XXVI
b) reacting the compound of Formula XXVI with p-nitrophenyl chloroformate to give a compound of Formula XXXI 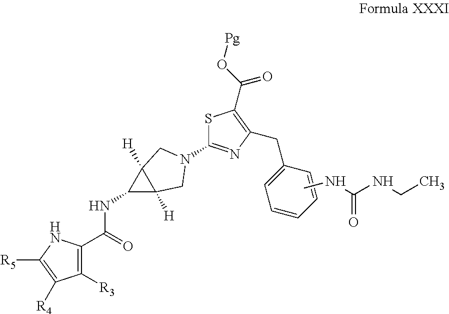
c) hydrolyzing the compound of Formula XXXI to give a compound of Formula XXXII.
wherein
R₃, R₄, R₅ are independently hydrogen, alkyl, alkenyl, alkynyl, halogen, cyano, amino, hydroxy, alkoxy, carbonyl, thiocarbonyl, oxo, cycloalkyl, aryl, heteroaryl, heterocyclyl, arylalkyl, heteroarylalkyl, cycloalkylalkyl or heterocyclylalkyl or R₃ and R₄ or R₄ and R₅ taken together with the carbon atoms to which they are attached can form an aromatic or non-aromatic ring, which optionally contain heteroatom selected from N, O and S;
P_{g} is an alkyl group selected from methyl, ethyl or t-butyl.
